(19) United States
(12) Patent Application Publication
(10) Pub. No.: US 2024/0191309 A1
(43) Pub. Date: Jun. 13, 2024

(54) GENE CLASSIFIERS AND USES THEREOF

(71) Applicant: DERMTECH, INC., La Jolla, CA (US)

(72) Inventors: John William WHITAKER, La Jolla, CA (US); Michael HOWELL, La Jolla, CA (US); Rizi AI, La Jolla, CA (US); James ROCK, La Jolla, CA (US); Loren CLARKE, La Jolla, CA (US); Lien Thi VO, La Jolla, CA (US); Emad BAHRAMI SAMANI, La Jolla, CA (US); John Daniel DOBAK, III, La Jolla, CA (US); Jordan MCGHEE, La Jolla, CA (US); Zuxu YAO, La Jolla, CA (US)

(21) Appl. No.: 18/555,195

(22) PCT Filed: Apr. 12, 2022
(86) PCT No.: PCT/US2022/024488

§ 371 Date: Oct. 12, 2023

Related U.S. Application Data
(60) Provisional application No. 63/174,345, filed on Apr. 13, 2021.
(60) Provisional application No. 63/175,514, filed on Apr. 15, 2021.
(60) Provisional application No. 63/245,118, filed on Sep. 16, 2021.

(51) Int. Cl. C12Q 1/6886; C12Q 1/6851

(52) U.S. Cl. CPC C12Q 1/6886; C12Q 1/6851; C12Q 2600/112; C12Q 2600/158

(57) ABSTRACT

Described herein are methods, systems, and compositions for non-invasively diagnosing or detecting a skin disease or disorder. Diagnosing or detecting a non-melanoma skin cancer (e.g., squamous cell carcinoma or basal cell carcinoma) as provided herein comprises detecting and/or comparing gene expression levels of identified genes. Methods, systems, and compositions are also described for differentiation of non-melanoma skin cancer (NMSC) from other diseases, such as actinic keratosis (AK).

### CROSS-REFERENCE

This application claims the benefit of U.S. provisional patent application No. 63/174,345 filed on Apr. 13, 2021; U.S. provisional patent application No. 63/175,514 filed on Apr. 15, 2021; and U.S. provisional patent application No. 63/245,118 filed on Sep. 16, 2021, each of which are incorporated by reference in its entirety.

### BACKGROUND

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability. Existing methods for identifying skin diseases (such as cancer) suffer from invasiveness, low sensitivity, high cost, extended analysis times, or late-stage detection. Therefore, there exists a need in the art for non-invasive methods of assessing skin disease risk and providing early treatment interventions to prevent such diseases from manifesting.

### SUMMARY

Provided herein are methods and systems for analysis of non-melanoma skin disorders.

Provided herein are methods for non-invasive gene expression analysis of one or more target genes, comprising: receiving or obtaining data comprising measurements of RNA and/or DNA obtained from a skin sample using a non-invasive sampling method, the skin sample having been obtained from a subject suspected of having a one or more skin diseases, wherein the skin disease comprises SCC (squamous cell carcinoma) and/or BCC (basal cell carcinoma); and applying one or more classifiers to the measurements to analyze gene expression of the one or mor target genes in the skin sample suspected of comprising the one or more skin diseases. Further provided herein are methods wherein SCC comprises squamous cell carcinoma in situ (isSCC) or invasive squamous cell carcinoma (ivSCC). Further provided herein are methods wherein the non-invasive sampling method comprises adhering one or more adhesive patches to a skin area of the subject. Further provided herein are methods wherein the one or more adhesive patches are applied to the same area of skin of the subject. Further provided herein are methods wherein the one or more adhesive patches are applied to different areas of skin of the subject. Further provided herein are methods wherein the one or more adhesive patches are applied to about the same area of skin of the subject. Further provided herein are methods wherein the one or more adhesive patches are applied to a lesion. Further provided herein are methods wherein the DNA comprises gDNA or a complementary cDNA. Further provided herein are methods wherein the method comprises obtaining the measurements by performing RNA-sequencing (RNA-seq). Further provided herein are methods wherein the method comprises obtaining the measurements by RT-qPCR. Further provided herein are methods wherein the RNA comprises an mRNA. Further provided herein are methods wherein the mRNA encodes a protein that plays a role in one or more of keratinization, developmental biology, metabolism of vitamins or cofactors, neutrophil degranulation, NOD-like receptor signaling, or an innate immune system. Further provided herein are methods wherein the mRNA comprises transcripts of one or more of: MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. Further provided herein are methods wherein the mRNA comprises transcripts of one or more of: MT-ND3, JPH3, TIMP2, MT-ND4, SOX11, and MED16. Further provided herein are methods wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are methods wherein the mRNA comprises a transcript of AD000090.1, CASP14, CXCL8, FABP5, IVNS1ABP, KRT1, KRT2, KRT5, KRT6C, NAMPT, or SLC2A3. Further provided herein are methods wherein the mRNA comprises a transcript of CAPZA2, IVNS1ABP, NCOA1, RAB5B, SCAF11, STK4, or TPM4. Further provided herein are methods wherein the mRNA is indicative of SCC. Further provided herein are methods wherein the expression level of a gene encoded by the mRNA is increased relative to a control. Further provided herein are methods wherein the expression level of a gene encoded by the mRNA is increased at least 1.5 fold relative to a control. Further provided herein are methods wherein the expression level of a gene encoded by the mRNA has a p value of no more than 0.1 relative to a control. Further provided herein are methods wherein the expression level of a gene encoded by the mRNA has a p value of no more than 0.01 relative to a control. Further provided herein are methods wherein the SCC comprises squamous cell carcinoma in situ (isSCC) and/or invasive squamous cell carcinoma (ivSCC). Further provided herein are methods wherein the method further comprises providing an SCC treatment or an actinic keratosis (AK) treatment to a patient diagnosed with SCC or AK, respectively. Further provided herein are methods wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. Further provided herein are methods wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. Further provided herein are methods wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are methods wherein the mRNA comprises transcripts of one or more of RNA5SP202, MIR4421, HAL, RNA5SP263, TIMP1, and RNA5SP481. Further provided herein are methods wherein the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. Further provided herein are methods wherein the method further comprises diagnosing a patient identified as having the skin disease. Further provided herein are methods wherein the method further comprises providing a treatment to the subject diagnosed with the skin disease. Further provided herein are methods wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications. Further provided herein are methods wherein topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. Further provided herein are methods wherein immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

Provided herein are methods for non-invasive gene expression analysis of one or more target genes, comprising: receiving or obtaining data comprising measurements of RNA and/or DNA obtained from a skin sample using a non-invasive sampling method, the skin sample having been obtained from a subject suspected of having SCC, wherein the SCC comprises squamous cell carcinoma in situ (isSCC) or invasive squamous cell carcinoma (ivSCC); and applying a classifier to the measurements to analyze gene expression of the one or more target genes in the skin sample suspected of comprising isSCC or ivSCC. Further provided herein are methods the method comprising obtaining the skin sample by adhering the adhesive patch to a skin area of the subject. Further provided herein are methods wherein the DNA comprises gDNA or a complementary cDNA. Further provided herein are methods comprising obtaining the measurements by performing RNA-sequencing (RNA-seq). Further provided herein are methods wherein the method comprises obtaining the measurements by RT-qPCR. Further provided herein are methods wherein the RNA comprises mRNA. Further provided herein are methods wherein the mRNA comprises transcripts of one or more of: AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP4-6, KRTAP9-4, MAGED1, MT-CO2, PIM1, RN7SL752P, SERPINA1, SLC2A3, SPRR2A, SRGN, or U3. Further provided herein are methods wherein the mRNA comprises a transcript of AC105460.1, ARC, COL6A1, EDN1, FAM110C, FOSL1, IL36G, JUN, PIM1, or RN7SL752P. Further provided herein are methods wherein the mRNA comprises a transcript of CYFIP1, DDX3X, ITGA3, MAGED1, MT-CO2, or SPRR2A. Further provided herein are methods wherein the mRNA is indicative of isSCC. Further provided herein are methods wherein the mRNA comprises a transcript of C5AR1, G0S2, KRTAP1-3, KRTAP1-5, KRTAP3-1, KRTAP4-6, KRTAP9-4, SERPINA1, SLC2A3, or SRGN. Further provided herein are methods wherein the mRNA comprises a transcript of EFCAB2, KRTAP4-6, KRTAP9-4, or U3. Further provided herein are methods wherein the mRNA is indicative of ivSCC. Further provided herein are methods wherein the expression level of a gene encoded by the mRNA is increased at least 1.5 fold relative to a control. Further provided herein are methods wherein the gene encoded by the mRNA has a p-value of no more than 0.1 relative to a control. Further provided herein are methods wherein the gene encoded by the mRNA has a p-value of no more than 0.01 relative to a control. Further provided herein are methods wherein the method further comprises providing an isSCC treatment or an ivSCC treatment to a subject diagnosed with isSCC or ivSCC. Further provided herein are methods wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications. In some instances, topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. In some embodiments, immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

Provided herein are methods for non-invasive differential gene expression analysis of one or more target genes between SCC and one or more different diseases, comprising: receiving or obtaining data comprising measurements of RNA and/or DNA obtained from a skin sample using a non-invasive sampling method, the skin sample having been obtained from a subject suspected of having SCC (squamous cell carcinoma) or the one or more different diseases; and applying one or more classifiers to the measurements to analyze gene expression of the one or more target genes in the skin sample indicative of SCC and/or the one or more different skin diseases. Further provided herein are methods wherein the one or more different diseases comprises AK. Further provided herein are methods wherein the one or more different diseases comprises one or more diseases in Table 10. Further provided herein are methods wherein the RNA comprises mRNA. Further provided herein are methods wherein the method comprises obtaining the measurements by RT-qPCR. Further provided herein are methods wherein at least one mRNA transcript in the sample comprising SCC is upregulated compared to a control sample comprising the different disease. Further provided herein are methods wherein the mRNA comprises a transcript of one or more of IVNS1ABP, RAB5B, CAPZA2, STK4, SCAF11, NCOA1, and CBL. Further provided herein are methods wherein at least one mRNA transcript in the sample comprising SCC is downregulated compared to a control sample comprising the different disease. Further provided herein are methods wherein at least one mRNA transcript in the sample comprising ivSCC is upregulated compared to a control sample comprising isSCC. Further provided herein are methods wherein the mRNA comprises transcripts of one or more of: MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. Further provided herein are methods wherein the mRNA comprises transcripts of one or more of: MT-ND3, JPH3, TIMP2, MT-ND4, SOX11, and MED16. Further provided herein are methods wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are methods wherein the mRNA comprises a transcript of AD000090.1, CASP14, CXCL8, FABP5, IVNS1ABP, KRT1, KRT2, KRT5, KRT6C, NAMPT, or SLC2A3. Further provided herein are methods wherein the mRNA comprises a transcript of CAPZA2, IVNS1ABP, NCOA1, RAB5B, SCAF11, STK4, or TPM4. Further provided herein are methods wherein the mRNA comprises EFCAB2, KRTAP4-6, KRTAP9-4, and U3. Further provided herein are methods wherein the mRNA comprises transcripts of one or more of RNA5SP202, MIR4421, HAL, RNA5SP263, TIMP1, and RNA5SP481. Further provided herein are methods wherein at least one mRNA transcript in the sample comprising ivSCC is downregulated compared to a control sample comprising isSCC. Further provided herein are methods wherein the mRNA comprises a transcript of one or more of DDX3X, CYFIP1, ITGA3, MAGED1, SPRR2Am, MT-C02. Further provided herein are methods wherein the expression level of a gene encoded by the mRNA is increased at least 1.5 fold relative to a control. Further provided herein are methods wherein the gene encoded by the mRNA has a p-value of no more than 0.1 relative to a control. Further provided herein are methods wherein the gene encoded by the mRNA has a p-value of no more than 0.01 relative to a control.

Provided herein are methods for non-invasive differential gene expression analysis of one or more target genes between basal cell carcinoma (BCC) and one or more different diseases, comprising: receiving or obtaining data comprising measurements of RNA and/or DNA obtained from a skin sample using a non-invasive sampling method, the skin sample having been obtained from a subject suspected of having BCC (basal cell carcinoma) or the one or more different diseases; and applying one or more classifiers to the measurements to analyze gene expression of the one or more target genes in the skin sample indicative of BCC and/or the one or more different skin diseases. Further provided herein are methods wherein the one or more different diseases comprises AK. Further provided herein are methods wherein the one or more different diseases comprises one or more diseases in Table 10. Further provided herein are methods wherein the RNA comprises mRNA. Further provided herein are methods wherein the method comprises obtaining the measurements by RT-qPCR. Further provided herein are methods wherein at least one mRNA transcript in the sample comprising BCC is upregulated compared to a control sample comprising the different disease. Further provided herein are methods wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. Further provided herein are methods wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. Further provided herein are methods wherein the mRNA comprises transcripts of one or more of: KRT85, TIMP1, FP236383.5, LCE6A, LCE2C, LCE1F, FP236383.4, IL37, LCE5A, AL139247.1, WARS1, and GBP1. Further provided herein are methods wherein the mRNA comprises a transcript of one or more of IVNS1ABP, RAB5B, CAPZA2, STK4, SCAF11, NCOA1, and CBL. Further provided herein are methods wherein the mRNA comprises transcripts of one or more of RNA5SP202, MIR4421, HAL, RNA5SP263, TIMP1, and RNA5SP481. Further provided herein are methods wherein at least one mRNA transcript in the sample comprising BCC is downregulated compared to a control sample comprising the different disease. Further provided herein are methods wherein the expression level of a gene encoded by the mRNA is increased at least 1.5 fold relative to a control. Further provided herein are methods wherein the expression level of a gene encoded by the mRNA has a p value of no more than 0.1 relative to a control. Further provided herein are methods wherein the expression level of a gene encoded by the mRNA has a p value of no more than 0.01 relative to a control.

Provided herein are non-invasive systems for analyzing a sample for differential gene expression, comprising: a communication interface that receives data comprising measurements of RNAs over a communication network, the measurements having been obtained from a skin sample using a non-invasive sampling method, and the skin sample having been obtained from a subject suspected of having SCC comprises squamous cell carcinoma in situ (isSCC) or invasive squamous cell carcinoma (ivSCC); and a computer in communication with the communication interface, wherein the computer comprises one or more computer processors and computer readable medium comprising machine-executable code that, upon execution by the one or more computer processors, implements a method comprising: applying a classifier to the measurements to identify the skin sample indicative of the isSCC or ivSCC. Further provided herein are systems for non-invasive gene expression analysis of one or more target genes, comprising: receiving or obtaining data comprising measurements of RNA and/or DNA obtained from a skin sample using a non-invasive sampling system, the skin sample having been obtained from a subject suspected of having SCC, wherein the SCC comprises squamous cell carcinoma in situ (isSCC) or invasive squamous cell carcinoma (ivSCC); and applying a classifier to the measurements to analyze gene expression of the one or more target genes in the skin sample suspected of comprising isSCC or ivSCC. Further provided herein are systems the system comprising obtaining the skin sample by adhering the adhesive patch to a skin area of the subject. Further provided herein are systems wherein the DNA comprises gDNA or a complementary cDNA. Further provided herein are systems comprising obtaining the measurements by performing RNA-sequencing (RNA-seq). Further provided herein are systems wherein the system comprises obtaining the measurements by RT-qPCR. Further provided herein are systems wherein the RNA comprises mRNA. Further provided herein are systems wherein the mRNA comprises transcripts of one or more of: AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP4-6, KRTAP9-4, MAGED1, MT-CO2, PIM1, RN7SL752P, SERPINA1, SLC2A3, SPRR2A, SRGN, or U3. Further provided herein are systems wherein the mRNA comprises a transcript of AC105460.1, ARC, COL6A1, EDN1, FAM110C, FOSL1, IL36G, JUN, PIM1, or RN7SL752P. Further provided herein are systems wherein the mRNA comprises a transcript of CYFIP1, DDX3X, ITGA3, MAGED1, MT-CO2, or SPRR2A. Further provided herein are systems wherein the mRNA is indicative of isSCC. Further provided herein are systems wherein the mRNA comprises a transcript of C5AR1, G0S2, KRTAP1-3, KRTAP1-5, KRTAP3-1, KRTAP4-6, KRTAP9-4, SERPINA1, SLC2A3, or SRGN. Further provided herein are systems wherein the mRNA comprises a transcript of EFCAB2, KRTAP4-6, KRTAP9-4, or U3. Further provided herein are systems wherein the mRNA is indicative of ivSCC. Further provided herein are systems wherein the expression level of a gene encoded by the mRNA is increased at least 1.5 fold relative to a control. Further provided herein are systems wherein the gene encoded by the mRNA has a p-value of no more than 0.1 relative to a control. Further provided herein are systems wherein the gene encoded by the mRNA has a p-value of no more than 0.01 relative to a control. Further provided herein are systems wherein the system further comprises providing an isSCC treatment or an ivSCC treatment to a subject diagnosed with isSCC or ivSCC. Further provided herein are systems wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications. In some instances, topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. In some embodiments, immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

Provided herein are non-invasive systems for analyzing a sample for differential gene expression comprising: providing a skin sample from a subject suspected of having one or more skin conditions, wherein the sample was obtained using a non-invasive sampling method; enriching RNAs from the skin sample; obtaining data comprising measurements from the RNAs; determining expression levels for one or more genes from the measurements; and applying a gene classifier to identify the skin sample comprises the one more skin conditions, wherein the skin disease comprises SCC (squamous cell carcinoma) BCC (basal cell carcinoma), AK (actinic keratosis), or a disease in Table 10. Further provided herein are systems wherein SCC comprises squamous cell carcinoma in situ (isSCC) or invasive squamous cell carcinoma (ivSCC). Further provided herein are systems wherein the non-invasive sampling system comprises adhering one or more adhesive patches to a skin area of the subject. Further provided herein are systems wherein the one or more adhesive patches are applied to the same area of skin of the subject. Further provided herein are systems wherein the one or more adhesive patches are applied to different areas of skin of the subject. Further provided herein are systems wherein the one or more adhesive patches are applied to about the same area of skin of the subject. Further provided herein are systems wherein the one or more adhesive patches are applied to a lesion. Further provided herein are systems wherein the DNA comprises gDNA or a complementary cDNA. Further provided herein are systems wherein the system comprises obtaining the measurements by performing RNA-sequencing (RNA-seq). Further provided herein are systems wherein the system comprises obtaining the measurements by RT-qPCR. Further provided herein are systems wherein the RNA comprises an mRNA. Further provided herein are systems wherein the mRNA encodes a protein that plays a role in one or more of keratinization, developmental biology, metabolism of vitamins or cofactors, neutrophil degranulation, NOD-like receptor signaling, or an innate immune system. Further provided herein are systems wherein the mRNA comprises transcripts of one or more of: MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. Further provided herein are systems wherein the mRNA comprises transcripts of one or more of: MT-ND3, JPH3, TIMP2, MT-ND4, SOX11, and MED16. Further provided herein are systems wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are systems wherein the mRNA comprises a transcript of AD000090.1, CASP14, CXCL8, FABP5, IVNS1ABP, KRT1, KRT2, KRT5, KRT6C, NAMPT, or SLC2A3. Further provided herein are systems wherein the mRNA comprises a transcript of CAPZA2, IVNS1ABP, NCOA1, RAB5B, SCAF11, STK4, or TPM4. Further provided herein are systems wherein the mRNA is indicative of SCC. Further provided herein are systems wherein the expression level of a gene encoded by the mRNA is increased relative to a control. Further provided herein are systems wherein the expression level of a gene encoded by the mRNA is increased at least 1.5 fold relative to a control. Further provided herein are systems wherein the expression level of a gene encoded by the mRNA has a p value of no more than 0.1 relative to a control. Further provided herein are systems wherein the expression level of a gene encoded by the mRNA has a p value of no more than 0.01 relative to a control. Further provided herein are systems wherein the SCC comprises squamous cell carcinoma in situ (isSCC) and/or invasive squamous cell carcinoma (ivSCC). Further provided herein are systems wherein the system further comprises providing an SCC treatment or an actinic keratosis (AK) treatment to a patient diagnosed with SCC or AK, respectively. Further provided herein are systems wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. Further provided herein are systems wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. Further provided herein are systems wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are systems wherein the mRNA comprises transcripts of one or more of RNA5SP202, MIR4421, HAL, RNA5SP263, TIMP1, and RNA5SP481. Further provided herein are systems wherein the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. Further provided herein are systems wherein the system further comprises diagnosing a patient identified as having the skin disease. Further provided herein are systems wherein the system further comprises providing a treatment to the subject diagnosed with the skin disease. Further provided herein are systems wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications. Further provided herein are systems wherein topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. Further provided herein are systems wherein immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

Provided herein are non-invasive systems for analyzing a sample for differential gene expression, comprising a communication interface that receives data comprising measurements of RNAs over a communication network, the measurements having been obtained from a skin sample using a non-invasive sampling method, and the skin sample having been obtained from a subject suspected of having one or more skin conditions; and a computer in communication with the communication interface, wherein the computer comprises one or more computer processors and computer readable medium comprising machine-executable code that, upon execution by the one or more computer processors, implements a method comprising: applying a classifier to the measurements to identify the skin sample indicative of the one or more skin conditions, wherein the skin disease comprises SCC (squamous cell carcinoma) BCC (basal cell carcinoma), AK (actinic keratosis), or a disease in Table 10. Further provided herein are systems wherein SCC comprises squamous cell carcinoma in situ (isSCC) or invasive squamous cell carcinoma (ivSCC). Further provided herein are systems wherein the non-invasive sampling system comprises adhering one or more adhesive patches to a skin area of the subject. Further provided herein are systems wherein the one or more adhesive patches are applied to the same area of skin of the subject. Further provided herein are systems wherein the one or more adhesive patches are applied to different areas of skin of the subject. Further provided herein are systems wherein the one or more adhesive patches are applied to about the same area of skin of the subject. Further provided herein are systems wherein the one or more adhesive patches are applied to a lesion. Further provided herein are systems wherein the DNA comprises gDNA or a complementary cDNA. Further provided herein are systems wherein the system comprises obtaining the measurements by performing RNA-sequencing (RNA-seq). Further provided herein are systems wherein the system comprises obtaining the measurements by RT-qPCR. Further provided herein are systems wherein the RNA comprises an mRNA. Further provided herein are systems wherein the mRNA encodes a protein that plays a role in one or more of keratinization, developmental biology, metabolism of vitamins or cofactors, neutrophil degranulation, NOD-like receptor signaling, or an innate immune system. Further provided herein are systems wherein the mRNA comprises transcripts of one or more of: MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. Further provided herein are systems wherein the mRNA comprises transcripts of one or more of: MT-ND3, JPH3, TIMP2, MT-ND4, SOX11, and MED16. Further provided herein are systems wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are systems wherein the mRNA comprises a transcript of AD000090.1, CASP14, CXCL8, FABP5, IVNS1ABP, KRT1, KRT2, KRT5, KRT6C, NAMPT, or SLC2A3. Further provided herein are systems wherein the mRNA comprises a transcript of CAPZA2, IVNS1ABP, NCOA1, RAB5B, SCAF11, STK4, or TPM4. Further provided herein are systems wherein the mRNA is indicative of SCC. Further provided herein are systems wherein the expression level of a gene encoded by the mRNA is increased relative to a control. Further provided herein are systems wherein the expression level of a gene encoded by the mRNA is increased at least 1.5 fold relative to a control. Further provided herein are systems wherein the expression level of a gene encoded by the mRNA has a p value of no more than 0.1 relative to a control. Further provided herein are systems wherein the expression level of a gene encoded by the mRNA has a p value of no more than 0.01 relative to a control. Further provided herein are systems wherein the SCC comprises squamous cell carcinoma in situ (isSCC) and/or invasive squamous cell carcinoma (ivSCC). Further provided herein are systems wherein the system further comprises providing an SCC treatment or an actinic keratosis (AK) treatment to a patient diagnosed with SCC or AK, respectively. Further provided herein are systems wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. Further provided herein are systems wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. Further provided herein are systems wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Further provided herein are systems wherein the mRNA comprises transcripts of one or more of RNA5SP202, MIR4421, HAL, RNA5SP263, TIMP1, and RNA5SP481. Further provided herein are systems wherein the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. Further provided herein are systems wherein the system further comprises diagnosing a patient identified as having the skin disease. Further provided herein are systems wherein the system further comprises providing a treatment to the subject diagnosed with the skin disease. Further provided herein are systems wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications. Further provided herein are systems wherein topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. Further provided herein are systems wherein immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

### BRIEF DESCRIPTION OF THE DRAWINGS

Various aspects of the disclosure are set forthwith particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C illustrate next generation sequencing quality metrics for 89 non-invasively obtained skin samples. FIG. 1A illustrates input reads (y-axis, millions, 0-150 at 50 unit intervals) for all samples (x-axis). FIG. 1B illustrates uniquely mapped reads (y-axis, millions, 0-90 at 30 unit intervals) for all samples (x-axis). FIG. 1C illustrates mapping statistics (%, 0-100 at 25% intervals) for all samples (x-axis). The three regions (top to bottom on graph) are unmapped %, duplicate %, and uniquely mapped %. The duplicate % was 14.62%+/−8.32%. Samples 26id46 (left) and 38id71 (right) are individually labeled.

FIG. 1D depicts intergenic reads indicative of genomic DNA (gDNA) contamination in a CDNA library. The y-axis is labeled intergen (%, 0-40 at 10% intervals) and the x-axis is labeled sample.

FIG. 1E depicts a 44 kb visualization window of reads in human (hg38) chr12, showing gDNA contamination between coding regions KRT2 and KRT1. The upper x-axis represents genome position from 52650 kb to 52680 labeled at 10 kb intervals. The upper plot is labeled negative and the lower plot is labeled positive. Genomic positions of genes KRT2 and KRT1 are shown at the bottom of the figure.

FIG. 2 depicts a 2D plot of the distribution of sample groups AK (circle), isSCC (triangle), and ivSCC (square). The y-axis is labeled Dim2 (4.5%) from −200 to 100 at 100 unit intervals; the x-axis is labeled Dim1 (28.8%) from −300 to 200 at 100 unit intervals.

FIG. 3A depicts a 2D plot of gene expression for AK and SCC samples. 11 DEGs (FC>2 and adj p<0.05) having elevated expression levels in SCC vs. AK are labeled, which included KRT1, KRT6C, CASP14, FABP5, KRT2, KRT5, SLC2A3, IVNS1ABP, NAMPT, CXCL8, and AD000090.1 (upper right quadrant). The y-axis is labeled −log 10(Padj) from 0.0 to 2.0 at 0.5 unit intervals; the x-axis is labeled log 2FC from −2 to 4 at 2 unit intervals.

FIG. 3B depicts a 2D plot showing samples for individual patients did not group together. The y-axis is labeled Dim2(4.7%) from −5 to 10 at 5 unit intervals; the x-axis is labeled Dim1 (58.6%) from −30 to 0 at 10 unit intervals. AK groups are labeled with a circle, SCC groups are labeled with a triangle.

FIG. 3C depicts a heat map for differential gene expression levels in each of the samples (class) analyzed for AK (orange) vs. SCC (blue). Lighter yellow areas indicate lower values, darker blue values indicate higher values. The inset color key is labeled value from 0-15 in 5 unit increments. Genes labeled on the right (top to bottom) are KRT1, KRT6C, CASP14, FABP5, KRT2, KRT5, SLC2A3, IVNS1ABP, NAMPT, CXCL8, and AD000090.1.

FIG. 3D depicts a 2D plot showing individual patient grouping. Patients did not group together. The y-axis is labeled Dim2 (32.6%) from −2 to 4 at 2 unit intervals; the x-axis is labeled Dim1 (37.7%) from −6 to 2 at 2 unit intervals. AK groups are labeled with a filled circle, SCC groups are labeled with a filled triangle. Unfilled circle, triangle, and square markers indicate three individual patients.

FIG. 4A depicts a method of in-silico removal of gDNA from reads. Both exonic (above) and non-exonic reads (below) are quantified.

FIG. 4B depicts a method of in-silico removal of gDNA from reads. Non-exonic reads (red, left) are excluded from exonic reads (blue, right). The y-axis is labeled frequencies (0-200 at 50 unit intervals), and the x-axis is labeled log 2 RPKM (−10 to 10 at 5 unit intervals).

FIG. 5A depicts a 2D plot of gene expression for AK and SCC samples correcting for gDNA. 219 DEGs (FC>2 and adj p<0.05) having elevated expression levels in SCC vs. AK are labeled, which included IVNS1ABP, RAB5B, CAPZA2, STK4, SCAF11, NCOA1, and CBL (upper right quadrant). Six DEGS overlapped between the type sample types. The y-axis is labeled −log 10(Padj) from 0 to 20 at 10 unit intervals; the x-axis is labeled log 2FC from 0 to 20 at 10 unit intervals.

FIG. 5B depicts a 2D plot showing individual patient grouping, corrected for gDNA. Patients did not group together. The y-axis is labeled Dim2 (4.7%) from −5 to 10 at 5 unit intervals; the x-axis is labeled Dim1 (58.6%) from −30 to 0 at 10 unit intervals. AK groups are labeled with a circle, SCC groups are labeled with a triangle. Genes labeled on the right (top to bottom) are: CD82, ENAH, CORO1C, RNH1, GTF3C1, CUX1, RPS7, PRR5, RPS2, HSPA4, MACROH2A1, DDX3X, USP22, STAT3, SULF2, SLC7A8, EWSR1, SAMD9, PRR14L, VMP1, KDM7A, ZNFI85, WDR26, TMEM45A, ZMIZ1, GAS7, SREBF2, TMEM59, UBE2D3, DDX24, NSD1, ARHGEF12, MKRN1, TFRC, DNM2, FLII, ACADVL, CAST, MED13L, APLP2, ARRDC3, ATP6VIA, ACTR2, TRIM25, NIN, PACSIN2, GADD45B, LCP2, AL929288.1, S100A6, GCNT7, ARHGAP30, CCDC93, KRT6C, and KRT2.

FIG. 5C depicts a heat map for differential gene expression levels in each of the samples analyzed for AK (orange) vs. SCC (blue). Lighter yellow areas indicate lower values, darker blue values indicate higher values. The inset color key is labeled value from 0-15 in 5 unit increments (log 2 CPM).

FIG. 6A depicts a 2D plot of gene expression for isSCC and ivSCC samples. For SCC subtype isSCC, FOSL1, AC105460.1, RN7SL752P, FAM110C, JUN, PIM1, IL36G, EDN1, ARC, CO_6A1 had upregulated gene expression (upper left quadrant) relative to ivSCC. For SCC subtype ivSCC, SERPINA1, C5AR1, G0S2, KRTAP1-5, SLC2A3, KRTAP4-6, SRGN, KRTAP3-1, KRTAP9-4, and KRTAP1-3 had upregulated gene expression (upper right quadrant) relative to isSCC.

FIG. 6B depicts a 2D plot showing samples for individual patients did not group together. The y-axis is labeled Dim2 (12.5%) from −10 to 10 at 10 unit intervals; the x-axis is labeled Dim1 (39.6%) from −10 to 20 at 10 unit intervals. isSCC groups are labeled with a circle, ivSCC groups are labeled with a triangle.

FIG. 6C depicts a heat map for differential gene expression levels in each of the samples analyzed for isSCC (orange) vs. ivSCC (blue). Lighter yellow areas indicate lower values, darker blue values indicate higher values. The inset color key is labeled value from 0-15 in 5 unit increments (log 2 CPM). Genes labeled on the right of the heat map (top to bottom) are: DLGAP4, KLF4, KLF6, AKAP17A, TNC, TP53BP2, COL6A2, SLC5A3, TRPV3, MT-ND4L, HSPB1, TRIM29, KRT5, MT-ND5, 7SK, MT-ND2, GDA, COL6A1, ELK3, PHLDB3, KRT9, USP11, NGEF, MAGEF1, SERPINA1, G0S2, SDC1, OVOL1, CPEB2, UGCG, SPRR2B, ANXA8L1, ELOVL1, PHLDA2, ODC1, IL36G, EDF1, PLP2, ABHD11, SLC4A11, UBL4A, and KRTAP1-5, and KRTAP9-4.

FIG. 6D depicts a 2D plot showing individual patient grouping. Patients did not group together. The y-axis is labeled Dim2 (12.5%) from −10 to 10 at 10 unit intervals; the x-axis is labeled Dim1 (39.6%) from −10 to 20 at 10 unit intervals. isSCC groups are labeled with a circle, ivSCC groups are labeled with a triangle.

FIG. 7A depicts a 2D plot of gene expression for SCC subtype isSCC and ivSCC samples corrected for gDNA. 101 DEGs (FC>2 and adj p<0.05) having elevated expression levels in isSCC vs. ivSCC included IVNS1ABP, RAB5B, CAPZA2, STK4, SCAF11, NCOA1, and CBL. 25 DEGS were overlapping between the two samples. Labeled genes DDX3X, CYFIP1, ITGA3, MAGED1, SPRR2A, and MT-CO2 were downregulated in isSCC compared to ivSCC (upper left quadrant). Labeled genes EFCAB2, KRTAP4-6, KRTAP9-4, and U3 were upregulated in ivSCC compared to isSCC (upper right quadrant).

FIG. 7B depicts a 2D plot showing individual patient grouping, corrected for gDNA. Patients did not group together. The y-axis is labeled Dim2 (15%) from −5 to 5 at 5 unit intervals; the x-axis is labeled Dim1 (41.3%) from −5 to 20 at 5 unit intervals. isSCC groups are labeled with a circle, ivSCC groups are labeled with a triangle.

FIG. 7C depicts a heat map for differential gene expression levels in each of the samples analyzed for isSCC (orange) vs. ivSCC (blue). Lighter yellow areas indicate lower values, darker blue values indicate higher values. The inset color key is labeled value from 0-15 in 5 unit increments (log 2 CPM). Genes on the right of the heat map (top to bottom) are: SPRR2A, MT-ND3, ODC1, MTCO1P12, MTND1P23, RN7SKP71, MT-ATP6, MT-ND5, ATP2B4, NUP50, FOSB, RPL31, APBB2, CAMSAP1, EXPH5, BTF3, ACADVL, CDDC6, GUK1, CDC42SE1, DDX3X, HSPA9, ATP13A3, USP36, FLOT2, AC109517.1, AL158141.1, AL108462.1, ARC, PIK3C2A, AFF1, EFCAB2, ZNF320, and KRTAP9-4.

FIG. 8A depicts genes found significantly differentially expressed between BCC and AK/Other. The p-values are calculated from F-test ANOVA. For each NMSC type, the genes are sorted by p-value. For each graph, sample types on the x-axis (left to right) are labeled 1-Other, 2-AK, and 4-BCC. The y-axes of each graph are labeled log 2 (normalized CT+1) from 0 to 4 at 1 unit intervals.

FIG. 8B depicts further genes found significantly differentially expressed between BCC and AK/Other. The p-values are calculated from F-test ANOVA. For each NMSC type, the genes are sorted by p-value. For each graph, sample types on the x-axis (left to right) are labeled 1-Other, 2-AK, and 4-BCC. The y-axes of each graph are labeled log 2 (normalized CT+1) from 0 to 4 at 1 unit intervals.

FIG. 8C depicts a graph of ROC-AUC vs. genes for BCC vs. AK/Other. The y-axis is labeled ROC-AUC from 0.00 to 0.75 at 0.25 unit intervals, and the y-axis is labeled targets.

FIG. 9A depicts genes found significantly differentially expressed between SCC and AK/Other. The p-values are calculated from F-test ANOVA. For each NMSC type, the genes are sorted by p-value. For each graph, sample types on the x-axis (left to right) are labeled 1-Other, 2-AK, and 4-BCC. The y-axes of each graph are labeled log 2 (normalized CT+1) from 0 to 4 at 1 unit intervals.

FIG. 9B depicts genes found significantly differentially expressed between SCC and AK/Other. The p-values are calculated from F-test ANOVA. For each NMSC type, the genes are sorted by p-value. For each graph, sample types on the x-axis (left to right) are labeled 1-Other, 2-AK, and 4-BCC. The y-axes of each graph are labeled log 2 (normalized CT+1) from 0 to 4 at 1 unit intervals.

FIG. 9C depicts a graph of ROC-AUG vs. genes for SCC vs. AK/Other. The y-axis is labeled ROC-AUC from 0.00 to 0.75 at 0.25 unit intervals, and the y-axis is labeled targets.

FIG. 10A depicts a graph of principal component analysis of samples collected from lesional skin of BCC (n=58) and other non-cancerous skin diseases (n=42). The y-axis is labeled PC2: 13% variance from −40 to 20 at 20 unit intervals; the x-axis is labeled PC1: 53% variance from −50 to 50 at 50 unit intervals. The legend depicts groups Other (orange circles) and BCC (teal circles).

FIG. 10B depicts a heat map of gene expression of BCC (n=58, orange) compared with other non-cancerous skin diseases (n=42, teal). Blue areas indicate lower values, and red areas indicate higher values. The inset color key is labeled value from −4 to 8 in 2 unit increments. Genes on the right of the heat map (top to bottom) are: KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6.

FIG. 10C-10D depicts the top 12 genes capable of classifying BCC versus non-cancerous skin disease. FIG. 10C depicts six genes (left to right, top to bottom: KRT85, TIMP1, FP236383.5, LCE6A, LCE2C, LCE1F) and FIG. 10D depicts six genes (top to bottom, left to right: FP236383.4, IL37, LCE5A, AL139247.1, WARS1, GBP1). Each graph y-axis is labeled normalized count, and the x-axis is labeled group (left to right) Other or BCC.

FIG. 11A depicts a graph of principal component analysis of samples collected from lesional skin of SCC (n=41) and other non-cancerous skin diseases (n=42). The y-axis is labeled PC2: 14% variance from −20 to 20 at 20 unit intervals; the x-axis is labeled PC1: 51% variance from −50 to 50 at 50 unit intervals. The legend depicts groups Other (orange circles) and SCC (teal circles).

FIG. 11B depicts a heat map of gene expression of SCC (n=58, orange) compared with other non-cancerous skin diseases (n=42, blue). Blue areas indicate lower values, and red areas indicate higher values. The inset color key is labeled value from −4 to 8 in 2 unit increments. Genes on the right of the heat map (top to bottom) are: DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77.

FIG. 11C-11D depicts the top 12 genes capable of classifying SCC versus non-cancerous skin disease. FIG. 11C depicts six genes (left to right, top to bottom: KRT33A, KRTAP8.1, KRTAP19-5, KRTAP2-2, KRTAP11-1, RTAP9-4) and FIG. 11D depicts six genes (left to right, top to bottom: KRT33B, KRTAP1-5, KRTAP3-3, KRTAP2-1, KRTAP3-1, KRTAP1-1). Each graph y-axis is labeled normalized count, and the x-axis is labeled group (left to right) Other or SCC.

FIGS. 12A-12C depict heat maps of genes measured in BCC (FIG. 12A), SCC (FIG. 12B), and normal skin samples (FIG. 12C). The genes tested (left side of heat map, top to bottom) were RNA5SP202, MIR4421, HAL, RNA5SP263, TIMP1, and RNA5SP481.

FIG. 12D depicts plots for gene expression levels of normal, BCC, and SCC non-invasive skin samples. Genes examined include (left to right, top to bottom): MIR4421, RNA5SP263, RNA5SP481, TIMP1, and HAL. The y-axes are labeled gene expression from 0.1 to 100 at log intervals; the x-axes are labeled with sample type (left to right): normal, BCC, and SCC.

### DETAILED DESCRIPTION

Provided herein are methods and systems for using gene classifiers non-melanoma skin cancer (NMSC) encompasses a collection of skin cancers that is not melanoma and is the most common type of skin cancer. NMSC includes angiosarcoma, basal cell carcinoma (BCC), cutaneous B-cell lymphoma, cutaneous T-cell lymphoma, dermatofibrosarcoma protuberans, Merkel cell carcinoma, sebaceous carcinoma, and squamous cell carcinoma of the skin (SCC). In some instances, basal cell carcinoma and squamous cell carcinoma are the two most common types of NMSC. In some cases, methods and compositions as described herein comprises differentiating between skin samples having SCC and AK (actinic keratosis). In some cases, methods and compositions as described herein comprises differentiating between skin samples having isSCC (in situ squamous cell carcinoma) and ivSCC (invasive squamous cell carcinoma). In some instances, methods described herein are used to identify, detect, or diagnose skin conditions which present similar cutaneous manifestations.

Squamous cell carcinoma (SCC) (also known as cutaneous squamous cell carcinoma (CSCC) or epidermoid carcinoma) is the second most common form of skin cancer. Similar to BCC, SCC also originates from the basal keratinocytes and is a slow growing cancer, usually found in UV exposed areas such as the head and neck. In some instances, SCC is further classified into subtypes and the subtypes comprises squamous cell carcinoma in situ (also known as Bowen's disease or isSCC), invasive squamous cell carcinoma (SCCI/ivSCC), clear cell SCC, spindle cell (sarcomatoid) SCC, SCC with single cell infiltrates, de novo SCC, verrucous carcinoma (VC), and lymphoepithelioma-like carcinoma of the skin. In some instances, the SCC subtype also comprises keratoacanthomas.

Basal cell carcinoma (BCC) is an uncontrolled growth or lesion from the basal cells (or basal keratinocytes), the deepest layer of the epidermis. In some instances, BCC is developed on sun-exposed areas, e.g., in the head and neck area. BCC is a slow-growing cancer and generally does not spread to other parts of the body. In some instances, BCC is further classified into subtypes and the subtypes comprises nodular BCC (pigmented), superficial BCC (pigmented), infundibulocystic BCC, fibroepithelial BCC, morpheaform BCC, infiltrative BCC, micronodular BCC, basosquamous BCC, and perineural invasion (PNI). Additional subtypes of BCC include nodulocystic, microcystic, adenoid, follicular, rodent ulcer, neurotropic, solitary basal cell carcinoma in young persons, pleomorphic, clear cell, granular cell, and singlet ring cell BCC.

Diagnosis of skin cancers include both invasive techniques and non-invasive methods with the gold standard being biopsy followed by histopathology evaluation. In some instances, non-invasive methods have reduced specificity and/or sensitivity, and often require a biopsy step for conclusive diagnosis.

Provided herein are systems and methods for non-invasively diagnosing (or detecting or identifying) a skin disease, e.g., a non-melanoma skin cancer (NMSC) or melanoma. In some instances, methods and compositions as described herein are used for diagnosing or detecting BCC. In other instances, methods and compositions as described herein are used for diagnosing or detecting SCC. In other instances, methods and compositions as described herein are used for diagnosing or detecting ivSCC and/or isSCC. In some cases, methods and compositions as described herein comprise improved sensitivity and specificity for diagnosing or detecting a skin disease, e.g., a NMSC such as BCC or SCC. In some instances, non-invasive sampling comprises obtaining a skin sample with an adhesive patch. In some instances, systems and methods described herein comprise next generation sequencing. Further provided herein are methods and systems for differentiating BCC and/or SCC from another skin disease.

### Differential Gene Expression Assay

Methods and compositions as described herein are used for detecting gene expression levels of a gene of interest. In some instances, the gene of interest is one or more of implicated in a skin disease. In some instances, the skin disease is a non-melanoma skin cancer (NMSC). In some instances, the NMSC is BCC or SCC. In some cases, the skin disease is melanoma. In some instances, the SCC comprises ivSCC and/or isSCC. In some cases, the skin disease is melanoma. In some instances, exemplary genes comprise one or more of genes found in Table 4A. In some instances, exemplary genes comprise one or more of genes found in Table 5A. In some instances, exemplary genes comprise one or more of genes found in Table 6A. In some instances, exemplary genes comprise one or more of genes found in Table 7A. In some instances, exemplary genes comprise one or more of genes found in Table 8. In some instances, exemplary genes comprise one or more of genes found in Table 9.

Provided herein are methods and systems for differential gene expression. In some embodiments, gene expression analysis is used to differentiate between skin diseases or disorders. In some embodiments, gene expression analysis is used to differentiate between SCC and another skin disorder. In some embodiments, gene expression analysis is used to differentiate between BCC and another skin disorder. In some embodiments, the other skin disorder comprises actinic keratosis (AK). In some embodiments, the other skin disorder comprises psoriasis. In some embodiments, the other skin disorder comprises atopic dermatitis (AD). In some embodiments, the other skin disorder comprises melanoma. In some embodiments, the other skin disorder comprises one or more of seborrheic keratosis, verruca vulgaris, and plaque psoriasis. In some instances, the other skin disorder comprises one or more of Acantholytic Dyskeratotic acanthoma; Acanthoma Fissuratum; Angiofibroma; Benign Keratosis; Benign Adnexal Neoplasm with Follicular Differentiation; Benign Keratosis, ulcerated and inflamed; Benign Lichenoid Keratosis; Benign keratosis/Seborrheic keratosis, mildly inflamed; Benign lichenoid keratosis; Benign stasis dermatitis; Benign verrucous keratosis; Chronic Folliculitis; Consistent with Fibrous Papule; Consistent with Lichen Simplex Chronicus; Dermal Fibrosis and Vascular Telangiectasia; Dermal Scar; Dermatofibroma with Lentiginous epidermal hyperplasia and basilar hypermelanosis; Epidermal Cyst; Epidermal Inclusion Cyst at the site of a Seborrheic Keratosis; Epidermal and follicular acanthosis with lichenoid lymphocytic inflammation and an endophytic component; Epidermal inclusion cyst; Eroded and inflamed Benign Keratosis; Fibrosing Granulation Tissue/Seborrheic Keratosis; Fibrous papule; Fibrous papule of the nose; Flat Seborrheic Keratosis; Granulomatous dermatitis; Hypertrophic scar; Inflamed Seborrheic Keratosis; Inflamed and irritated verrucous keratosis; Inflamed seborrheic keratosis; Inflamed verrucous keratosis; Inflammation, scar and regeneration; Irritated Benign Keratosis with Lichenoid Inflammation; Keloidal Scar; Lichen simplex chronicus; Lichenoid Keratosis; Nodular acute and chronic lymphohistiocytic inflammation; Perifollicular non-caseating granulomatous inflammation with multinucleated giant cells; Porokeratosis; Psoriasiform dermatitis with focal intraepidermal atypical lymphocytes; Psoriasiform and Spongiotic dermatitis; Reticulated Seborrheic Keratosis; Scar; Scar with regeneration and repair; Scar with stasis changes; Sebaceous hyperplasia; Seborrheic Keratosis; Seborrheic Keratosis, clonal type; Seborrheic Keratosis, inflamed and irritated; Seborrheic Keratosis, pigmented and inflamed; Seborrheic keratosis, inflamed; Solar Lentigo; Spongiotic dermatitis; Spongiotic dermatitis and scar; Traumatized erosion with repair; Tumor of Follicular infundibulum; Verruca Vulgaris; Verruca Vulgaris Inflamed; Verrucous Keratosis, traumatized and inflamed; Verrucous keratosis; and Warty dyskeratoma (benign).

Provided herein are one or more genes for differential gene analysis. In some embodiments, exemplary genes are used for identifying SCC and/or BCC vs. other skin diseases. Exemplary genes associated with a skin disease (e.g., a NMSC such as BCC or SCC) and, in some instances, detected using methods described herein include, but are not limited to, IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. Exemplary genes associated with a skin disease (e.g., a NMSC such as BCC or SCC) and, in some instances, detected using methods described herein include, but are not limited to MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, And KRTAP1-1. Exemplary genes associated with a skin disease (e.g., a NMSC such as BCC or SCC) and, in some instances, detected using methods described herein include, but are not limited to MT-ND3, JPH3-053, TIMP2, MT-ND4, SOX11, MED16, KIF1B, MT-ND1, COL17A1, HERC4-778, AKR1C1, TAGLN, SLCO1A2, ZIC2, LINC02167, ATPAF2, PUM2, CYP24A1, CCT8, and MT-CYB. Exemplary genes associated with a skin disease (e.g., a NMSC such as BCC or SCC) and, in some instances, detected using methods described herein include, but are not limited to TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. Exemplary genes associated with a skin disease (e.g., a NMSC such as BCC or SCC) and, in some instances, detected using methods described herein include, but are not limited to TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP. Exemplary genes associated with a skin disease (e.g., a NMSC such as BCC or SCC) and, in some instances, detected using methods described herein include, but are not limited to KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6. Exemplary genes associated with a skin disease (e.g., a NMSC such as BCC or SCC) and, in some instances, detected using methods described herein include, but are not limited to KRT85, TIMP1, FP236383.5, LCE6A, LCE2C, LCE1F, FP236383.4, IL37, LCE5A, AL139247.1, WARS1, and GBP1. Exemplary genes associated with a skin disease (e.g., a NMSC such as BCC or SCC) and, in some instances, detected using methods described herein include, but are not limited to DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. Exemplary genes associated with a skin disease (e.g., a NMSC such as BCC or SCC) and, in some instances, detected using methods described herein include, but are not limited to KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-3, KRTAP2-1, KRTAP3-1, and KRTAP1-1. Exemplary genes associated with a skin disease (e.g., a NMSC such as BCC or SCC) and, in some instances, detected using methods described herein include, but are not limited to MT-ND3, JPH3, TIMP2, MT-ND4, SOX11, and MED16. Exemplary genes associated with a skin disease (e.g., a NMSC such as BCC or SCC) and, in some instances, detected using methods described herein include, but are not limited to TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. Exemplary genes associated with a skin disease (e.g., a NMSC such as BCC or SCC) and, in some instances, detected using methods described herein include, but are not limited to KRT85, TIMP1, FP236383.5, LCE6A, LCE2C, LCE1F, FP236383.4, IL37, LCE5A, AL139247.1, WARS1, and GBP1. Exemplary genes associated with a skin disease (e.g., a NMSC such as BCC or SCC) and, in some instances, detected using methods described herein include, but are not limited to MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1.

Provided herein are one or more genes for differential gene analysis. In some embodiments, exemplary genes are used for identifying BCC vs. other skin diseases. In some embodiments, exemplary genes expression levels are increased in a BCC sample relative to a control. In some instances, at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least three of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least four of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least five of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least six of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least seven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least eight of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least nine of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some instances, at least ten of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, KRT7, and SPPG1 are detected. In some instances, at least eleven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, KRT7, and SPPG1 are detected. In some instances, at least twelve of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, KRT7, and SPPG1 are detected. In some instances, all of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are detected. In some embodiments, the gene expression levels of one or more genes of interest are detected. In some instances, at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least three of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, and GBP are detected. In some instances, at least four of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least five of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least six of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least seven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least eight of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least nine of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least ten of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, and GBP are detected. In some instances, at least eleven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, at least twelve of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are detected. In some instances, all of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1 are detected. In some instances, one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, KRT7, and SPPG1 are upregulated in BCC vs. AK. In some instances, two or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 are upregulated in BCC vs. AK. In some instances, one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1 are upregulated in BCC vs. AK. In some instances, two or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP are upregulated in BCC vs. AK. In some embodiments, the gene expression levels of one or more genes of interest are detected. In some instances, at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least three of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least four of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least five of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least six of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least seven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least eight of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least nine of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least ten of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least eleven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, at least twelve of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, all of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are detected. In some instances, one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are upregulated in BCC vs. AK. In some instances, two or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 are upregulated in BCC vs. AK. In some embodiments, the gene expression levels of one or more genes of interest are detected. In some instances, at least two of KRT85, TIMP1, FP236383.5, LCE6A, LCE2C, LCE1F, FP236383.4, IL37, LCE5A, AL139247.1, WARS1, and GBP are detected. In some instances, at least three of KRT85, TIMP1, FP236383.5, LCE6A, LCE2C, LCE1F, FP236383.4, IL37, LCE5A, AL139247.1, WARS1, and GBP are detected. In some instances, at least four of KRT85, TIMP1, FP236383.5, LCE6A, LCE2C, LCE1F, FP236383.4, IL37, LCE5A, AL139247.1, WARS1, and GBP are detected. In some instances, at least five of KRT85, TIMP1, FP236383.5, LCE6A, LCE2C, LCE1F, FP236383.4, IL37, LCE5A, AL139247.1, WARS1, and GBP are detected. In some instances, at least six of KRT85, TIMP1, FP236383.5, LCE6A, LCE2C, LCE1F, FP236383.4, IL37, LCE5A, AL139247.1, WARS1, and GBP are detected. In some instances, at least seven of KRT85, TIMP1, FP236383.5, LCE6A, LCE2C, LCE1F, FP236383.4, IL37, LCE5A, AL139247.1, WARS1, and GBP are detected. In some instances, at least eight of KRT85, TIMP1, FP236383.5, LCE6A, LCE2C, LCE1F, FP236383.4, IL37, LCE5A, AL139247.1, WARS1, and GBP are detected. In some instances, at least nine of KRT85, TIMP1, FP236383.5, LCE6A, LCE2C, LCE1F, FP236383.4, IL37, LCE5A, AL139247.1, WARS1, and GBP are detected. In some instances, at least ten of KRT85, TIMP1, FP236383.5, LCE6A, LCE2C, LCE1F, FP236383.4, IL37, LCE5A, AL139247.1, WARS1, and GBP are detected. In some instances, at least eleven of KRT85, TIMP1, FP236383.5, LCE6A, LCE2C, LCE1F, FP236383.4, IL37, LCE5A, AL139247.1, WARS1, and GBP are detected. In some instances, at least twelve of KRT85, TIMP1, FP236383.5, LCE6A, LCE2C, LCE1F, FP236383.4, IL37, LCE5A, AL139247.1, WARS1, and GBP are detected. In some instances, all of KRT85, TIMP1, FP236383.5, LCE6A, LCE2C, LCE1F, FP236383.4, IL37, LCE5A, AL139247.1, WARS1, and GBP are detected. In some instances, one or more of KRT85, TIMP1, FP236383.5, LCE6A, LCE2C, LCE1F, FP236383.4, IL37, LCE5A, AL139247.1, WARS1, and GBP are upregulated in BCC vs. AK. In some instances, two or more of KRT85, TIMP1, FP236383.5, LCE6A, LCE2C, LCE1F, FP236383.4, IL37, LCE5A, AL139247.1, WARS1, and GBP1 are upregulated in BCC vs. AK. In some embodiments, the gene expression levels of one or more genes of interest are detected. In some instances, at least two of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 are detected. In some instances, at least three of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least four of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least five of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KR T28, RNF5, IGF2BP2, TIMP1, EDN1, and KRT16P6 are detected. In some instances, at least six of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least seven of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least eight of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least nine of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least ten of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least eleven of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP, AC104109.2, C2orf76, PSG7, PLCXD2, KRT28, RNF5, IGF2BP2, TIMP1, EDN1, and KRT16P6 are detected. In some instances, at least twelve of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 are detected. In some instances, all of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 are detected. In some instances, one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 are upregulated in BCC vs. AK. In some instances, two or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 are upregulated in BCC vs. AK.

Expression levels of one or more gene may be detected. In some embodiments, the gene expression levels of one or more genes of interest are detected. In some embodiments, the gene expression levels of one or more genes of interest are detected for use in identifying SCC vs. other skin diseases. In some embodiments, exemplary genes expression levels are increased in a SCC sample relative to a control. In some instances, at least two of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least three of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least four of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least five of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least six of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least seven of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least eight of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least nine of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least ten of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least eleven of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, at least twelve of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some instances, all of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 are detected. In some embodiments, the gene expression levels of one or more genes of interest are detected. In some instances, at least two of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP-3, KRTAP-5, KRTAP3-1, KRTAP are detected. In some instances, at least three of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least four of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP-5, KRTAP3-1, KRTAP are detected. In some instances, at least five of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least six of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least seven of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP-3, KRTAP-5, KRTAP3-1, KRTAP are detected. In some instances, at least eight of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least nine of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least ten of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least eleven of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, at least twelve of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, all of AD000090.1, CASP14, CXCL8, FABP5, IVNS1ABP, KRT1, KRT2, KRT5, KRT6C, NAMPT, NCOA1, RAB5B, SCAF11, SLC2A3, STK4, TPM4, AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP are detected. In some instances, one or more of IVNS1ABP, RAB5B, CAPZA2, STK4, SCAF11, NCOA1, and CBL are upregulated in SCC vs. AK. In some instances, one or more of EFCAB2, KRTAP4-6, KRTAP9-4, and U3 are upregulated in isSCC vs. ivSCC. In some instances, one or more of DDX3X, CYFIP1, ITGA3, MAGED1, SPRR2Am, MT-CO2, RNA5SP202, MIR4421, HAL, RNA5SP263, TIMP1, and RNA5SP481 are downregulated in isSCC vs. ivSCC. In some instances, two or more of IVNS1ABP, RAB5B, CAPZA2, STK4, SCAF11, NCOA1, and CBL are upregulated in SCC vs. AK. In some instances, two or more of EFCAB2, KRTAP4-6, KRTAP9-4, and U3 are upregulated in isSCC vs. ivSCC. In some instances, one or more of DDX3X, CYFIP1, ITGA3, MAGED1, SPRR2Am, MT-CO2, RNA5SP202, MIR4421, HAL, RNA5SP263, TIMP1, and RNA5SP481 are downregulated in isSCC vs. ivSCC. In some instances, three or more of IVNS1ABP, RAB5B, CAPZA2, STK4, SCAF11, NCOA1, and CBL are upregulated in SCC vs. AK. In some instances, three or more of EFCAB2, KRTAP4-6, KRTAP9-4, and U3 are upregulated in isSCC vs. ivSCC. In some instances, three or more of DDX3X, CYFIP1, ITGA3, MAGED1, SPRR2Am, MT-CO2, RNA5SP202, MIR4421, HAL, RNA5SP263, TIMP1, and RNA5SP481 are downregulated in isSCC vs. ivSCC.

Expression levels of one or more gene may be detected. In some embodiments, the gene expression levels of one or more genes of interest are detected. In some embodiments, the gene expression levels of one or more genes of interest are detected for use in identifying SCC vs. other skin diseases. In some instances, at least two of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least three of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least four of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least five of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least six of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least seven of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least eight of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least nine of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least ten of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least eleven of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least twelve of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, all of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some embodiments, the gene expression levels of one or more genes of interest are detected. In some instances, one or more of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are upregulated in SCC vs. AK. In some embodiments, the gene expression levels of one or more genes of interest are detected for use in identifying SCC vs. other skin diseases. In some instances, at least two of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least three of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least four of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least five of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least six of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least seven of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least eight of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least nine of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least ten of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least eleven of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, at least twelve of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some instances, all of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are detected. In some embodiments, the gene expression levels of one or more genes of interest are detected. In some instances, one or more of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 are upregulated in SCC vs. AK. In some embodiments, the gene expression levels of one or more genes of interest are detected for use in identifying SCC vs. other skin diseases. In some instances, at least two of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least three of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least four of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least five of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least six of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least seven of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least eight of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least nine of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least ten of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least eleven of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, at least twelve of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some instances, all of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are detected. In some embodiments, the gene expression levels of one or more genes of interest are detected. In some instances, one or more of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, AND KRT77 are upregulated in SCC vs. AK. In some embodiments, the gene expression levels of one or more genes of interest are detected. In some embodiments, the gene expression levels of one or more genes of interest are detected for use in identifying SCC vs. other skin diseases. In some instances, at least two of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least three of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least four of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least five of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least six of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least seven of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least eight of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least nine of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least ten of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least eleven of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, at least twelve of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some instances, all of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are detected. In some embodiments, the gene expression levels of one or more genes of interest are detected. In some instances, one or more of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 are upregulated in SCC vs. AK. In some instances, one or more of MT-ND3, JPH3, TIMP2, MT-ND4, SOX11, and MED16 are upregulated in SCC vs. AK. In some embodiments, gene expression levels of one or more genes of interest comprise one or more of MT-ND3, JPH3, TIMP2, MT-ND4, SOX11, and MED16. In some embodiments, gene expression levels of one or more genes of interest comprise two or more of MT-ND3, JPH3-053, TIMP2, MT-ND4, SOX11, and MED16. In some embodiments, gene expression levels of one or more genes of interest comprise three or more of MT-ND3, JPH3, TIMP2, MT-ND4, SOX11, and MED16. In some embodiments, gene expression levels of one or more genes of interest comprise four or more of MT-ND3, JPH3, TIMP2, MT-ND4, SOX11, and MED16. In some embodiments, gene expression levels of one or more genes of interest comprise five or six of MT-ND3, JPH3, TIMP2, MT-ND4, SOX11, and MED16.

In some instances, BCC and/or SCC samples are compared to AK, SK, and normal samples. In some instances, BCC and SCC samples are compared to AK and SK samples. In some instances, BCC and SCC samples are compared to AK and normal samples. In some instances, BCC and SCC samples are compared SK and normal samples. In some instances, BCC and SCC samples are compared to AK samples. In some instances, BCC and SCC samples are compared to SK samples. In some instances, BCC and SCC samples are compared to normal samples. In some instances, BCC samples are compared to AK, SK, and normal samples. In some instances, BCC samples are compared to AK and normal samples. In some instances, BCC samples are compared to AK and SK samples. In some instances, BCC samples are compared to SK and normal samples. In some instances, BCC samples are compared to AK samples. In some instances, BCC samples are compared to SK samples. In some instances, BCC samples are compared to normal samples. In some instances, SCC samples are compared to AK, SK, and normal samples. In some instances, SCC samples are compared to AK and normal samples. In some instances, SCC samples are compared to AK and SK samples. In some instances, SCC samples are compared to SK and normal samples. In some instances, SCC samples are compared to AK samples. In some instances, SCC samples are compared to SK samples. In some instances, SCC samples are compared to normal samples. In some instances, BCC samples are compared to SCC samples.

In some embodiments, methods for differentiating cancer samples from non-cancer samples comprising detecting the gene expression levels of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof comprise improved specificity and sensitivity. In some embodiments, the specificity is at least or about 70%, 75%, 80%, 85%, 90%, or more than 95% when detecting the gene expression levels of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof. In some embodiments, the specificity is at least or about 70%, 75%, 80%, 85%, 90%, or more than 95% when detecting the gene expression levels of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof. In some embodiments, the specificity is at least or about 70%, 75%, 80%, 85%, 90%, or more than 95% when detecting the gene expression levels of IVNS1ABP, RAB5B, CAPZA2, STK4, SCAF1, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof. In some embodiments, methods for differentiating cancer samples from non-cancer samples comprising detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, KRTAP1-1 or a combination thereof comprise improved specificity and sensitivity. In some embodiments, the specificity is at least or about 70%, 75%, 80%, 85%, 90%, or more than 95% when detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, KRTAP1-1 or a combination thereof. In some embodiments, the specificity is at least or about 70%, 75%, 80%, 85%, 90%, or more than 95% when detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, KRTAP1-1, or a combination thereof. In some embodiments, the specificity is at least or about 70%, 75%, 80%, 85%, 90%, or more than 95% when detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP1-1, RTAP9-4, KRT33B, KRTAP1-5, KRTAP3-3, KRTAP2-1, KRTAP3-1, KRTAP1-1, or a combination thereof. In some embodiments, methods for differentiating cancer samples from non-cancer samples comprising detecting the gene expression levels of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof comprise improved specificity and sensitivity. In some embodiments, the specificity is at least or about 70%, 75%, 80%, 85%, 90%, or more than 95% when detecting the gene expression levels of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof. In some embodiments, the specificity is at least or about 70%, 75%, 80%, 85%, 90%, or more than 95% when detecting the gene expression levels of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof. In some embodiments, the specificity is at least or about 70%, 75%, 80%, 85%, 90%, or more than 95% when detecting the gene expression levels of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof. In some embodiments, the specificity is at least or about 70%, 75%, 80%, 85%, 90%, or more than 95% when detecting the gene expression levels of one or more of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP1-1, or a combination thereof. In some embodiments, the specificity is at least or about 70%, 75%, 80%, 85%, 90%, or more than 95% when detecting the gene expression levels of one or more of MT-ND3, JPH3, TIMP2, MT-ND4, SOX11, and MED16, or a combination thereof. In some embodiments, the specificity is at least or about 70%, 75%, 80%, 85%, 90%, or more than 95% when detecting the gene expression levels of one or more of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1, or a combination thereof. In some embodiments, the specificity is at least or about 70%, 75%, 80%, 85%, 90%, or more than 95% when detecting the gene expression levels of one or more of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77, or a combination thereof. In some embodiments, the specificity is at least or about 70%, 75%, 80%, 85%, 90%, or more than 95% when detecting the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1, or a combination thereof. In some embodiments, the specificity is at least or about 70%, 75%, 80%, 85%, 90%, or more than 95% when detecting the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1, or a combination thereof. In some embodiments, the specificity is at least or about 70%, 75%, 80%, 85%, 90%, or more than 95% when detecting the gene expression levels of one or more of KRT85, TIMP1, FP236383.5, LCE6A, LCE2C, LCE1F, FP236383.4, IL37, LCE5A, AL139247.1, WARS1, and GBP1, or a combination thereof. In some embodiments, the specificity is at least or about 70%, 75%, 80%, 85%, 90%, or more than 95% when detecting the gene expression levels of one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 or a combination thereof.

In some embodiments, the sensitivity is at least or about 70%, 75%, 80%, 85%, 90%, or more than 95% when detecting the gene expression levels of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof. In some embodiments, the sensitivity is at least or about 70%, 75%, 80%, 85%, 90%, or more than 95% when detecting the gene expression levels of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, AND RNA5SP481, UPP1, or a combination thereof. In some embodiments, the sensitivity is at least or about 70%, 75%, 80%, 85%, 90%, or more than 95% when detecting the gene expression levels of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof. In some embodiments, the sensitivity is at least or about 70%, 75%, 80%, 85%, 90%, or more than 95% when detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, KRTAP1-1, or a combination thereof. In some embodiments, the sensitivity is at least or about 70%, 75%, 80%, 85%, 90%, or more than 95% when detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1, or a combination thereof. In some embodiments, the sensitivity is at least or about 70%, 75%, 80%, 85%, 90%, or more than 95% when detecting the gene expression levels of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, KRTAP1-1, or a combination thereof. In some embodiments, the sensitivity is at least or about 70%, 75%, 80%, 85%, 90%, or more than 95% when detecting the gene expression levels of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof. In some embodiments, the sensitivity is at least or about 70%, 75%, 80%, 85%, 90%, or more than 95% when detecting the gene expression levels of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof. In some embodiments, the sensitivity is at least or about 70%, 75%, 80%, 85%, 90%, or more than 95% when detecting the gene expression levels of one or more of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP1-1, or a combination thereof. In some embodiments, the sensitivity is at least or about 70%, 75%, 80%, 85%, 90%, or more than 95% when detecting the gene expression levels of one or more of MT-ND3, JPH3, TIMP2, MT-ND4, SOX11, and MED16, or a combination thereof. In some embodiments, the sensitivity is at least or about 70%, 75%, 80%, 85%, 90%, or more than 95% when detecting the gene expression levels of one or more of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1, or a combination thereof. In some embodiments, the sensitivity is at least or about 70%, 75%, 80%, 85%, 90%, or more than 95% when detecting the gene expression levels of one or more of DSG4, GPRC5D, KRTAP4-11, KRTAP7-1, KRTAP8-1, KRTAP9-5, KRTAP9-1, KRTAP9-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77, or a combination thereof. In some embodiments, the sensitivity is at least or about 70%, 75%, 80%, 85%, 90%, or more than 95% when detecting the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1, or a combination thereof. In some embodiments, the sensitivity is at least or about 70%, 75%, 80%, 85%, 90%, or more than 95% when detecting the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1, or a combination thereof. In some embodiments, the sensitivity is at least or about 70%, 75%, 80%, 85%, 90%, or more than 95% when detecting the gene expression levels of one or more of KRT85, TIMP1, FP236383.5, LCE6A, LCE2C, LCE1F, FP236383.4, IL37, LCE5A, AL139247.1, WARS1, and GBP1, or a combination thereof. In some embodiments, the sensitivity is at least or about 70%, 75%, 80%, 85%, 90%, or more than 95% when detecting the gene expression levels of one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 or a combination thereof.

In some embodiments, cancer samples are differentiated from non-cancer samples when amplification of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof is detected. In some embodiments, amplification of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof is not detected in non-cancer samples. In some embodiments, cancer samples are differentiated from non-cancer samples when amplification of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, KRTAP1-1, or a combination thereof is detected. In some embodiments, amplification of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, KRTAP1-1, or a combination thereof is not detected in non-cancer samples. In some embodiments, cancer samples are differentiated from non-cancer samples when amplification of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof is detected. In some embodiments, amplification of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof is not detected in non-cancer samples. In some embodiments, cancer samples are differentiated from non-cancer samples when amplification of one or more of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1, or a combination thereof is detected. In some embodiments, cancer samples are differentiated from non-cancer samples when amplification of one or more of MT-ND3, JPH3, TIMP2, MT-ND4, SOX11, and MED16, or a combination thereof is detected. In some embodiments, cancer samples are differentiated from non-cancer samples when amplification of one or more of KRT33A, KRTAP8.1, KRTAP19-5, KRTAP2-2, KRTAP11-1, RTAP9-4, KRT33B, KRTAP-5, KRTAP3-3, KRTAP2-1, KRTAP3-1, and KRTAP1-1, or a combination thereof is detected. In some embodiments, cancer samples are differentiated from non-cancer samples when amplification of one or more of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77, or a combination thereof is detected. In some embodiments, cancer samples are differentiated from non-cancer samples when amplification of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1, or a combination thereof is detected. In some embodiments, cancer samples are differentiated from non-cancer samples when amplification of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1, or a combination thereof is detected. In some embodiments, cancer samples are differentiated from non-cancer samples when amplification of one or more of KRT85, TIMP1, FP236383.5, LCE6A, LCE2C, LCE1F, FP236383.4, IL37, LCE5A, AL139247.1, WARS1, and GBP1, or a combination thereof is detected. In some embodiments, cancer samples are differentiated from non-cancer samples when amplification of one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 or a combination thereof is detected.

Various methods for detecting gene expression levels are contemplated herein. In some instances, gene expression levels are quantified from the amount of RNA transcripts measured. For example, generating cDNA libraries and sequencing the libraries (e.g., RNA-seq). In some instances, RNA is isolated from a sample (e.g., non-invasive sample), reverse transcribed to generate cDNA, ligated to adapters, amplified, and sequenced. In some instances, the gene expression level is determined from the number of reads obtained for genes in the cDNA library. In some instances, the sequencing is performed using the Sanger sequencing method. In some instances, the sequencing involves the use of chain terminating dideoxynucleotides. In some instances, the sequencing involves gel-electrophoresis. In some instances, the sequencing is performed using a next generation sequencing method. In some instances, sequencing includes, but not limited to, single-molecule real-time (SMRT) sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, +S sequencing, and sequencing by synthesis.

In-silico methods may be used to reduce or eliminate gDNA from samples analyzed herein. In some instances, gDNA contaminates RNA, leading to artifactual changes in gene expression. In some instances, gDNA is removed prior to analysis (e.g., during RNA purification, library purification, or, or other wet lab step). In some instances, gDNA is removed in-silico, after obtaining expression data (such as by next generation sequencing). In some instances, exonic and non-exonic reads are quantified. In some instances, non-exonic reads are excluded from exonic reads. In some instances, gDNA removal methods result in a reduction of gDNA artifacts in the methods described herein.

RNA-seq may be used to detect genes and gene expression levels. For example, a cDNA library is used to detect at least two of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some cases, no more than 3, 4, 5, 6, 7, 8, 9, 10, 15, 20, or 22 genes are detected. In some instances, next generation sequencing is used to detect at least one of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least three of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least four of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least five of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least six of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least seven of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least eight of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least nine of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least ten of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least eleven of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect at least twelve of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, next generation sequencing is used to detect all of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. For example, a cDNA library is used to detect at least two of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some cases, no more than 3, 4, 5, 6, 7, 8, 9, 10, 15, 20, or 22 genes are detected. In some instances, next generation sequencing is used to detect at least one of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least three of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least four of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least five of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least six of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least seven of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least eight of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least nine of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least ten of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least eleven of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect at least twelve of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect all of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1. RNA-seq may be used to detect genes and gene expression levels. For example, a cDNA library is used to detect at least one of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In other embodiments, a cDNA library is used to detect at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some cases, no more than 3, 4, 5, 6, 7, 8, 9, 10, 15, 20, or 22 genes are detected. In some instances, next generation sequencing is used to detect at least three of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least four of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least five of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least six of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least seven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least eight of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, and KRT7. In some instances, next generation sequencing is used to detect at least nine of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least ten of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least eleven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect at least twelve of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect all of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, next generation sequencing is used to detect 1, 2, 3, 4, 5, 6, 7, 8, 9, 10, 11, 12, or more than 12 of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP-1. In some instances, next generation sequencing is used to detect 1, 2, 3, 4, 5, or 6 of MT-ND3, JPH3, TIMP2, MT-ND4, SOX11, and MED16. In some instances, next generation sequencing is used to detect 1, 2, 3, 4, 5, 6, 7, 8, 9, 10, 11, 12, or more than 12 of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1. In some instances, next generation sequencing is used to detect 1, 2, 3, 4, 5, 6, 7, 8, 9, 10, 11, 12, or more than 12 of DSG4, GPRC5D, KRTAP4-11, KRTAP7-1, KRTAP8-1, KRTAP9-5, KRTAP9-1, KRTAP9-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, next generation sequencing is used to detect 1, 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, next generation sequencing is used to detect 1, 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, next generation sequencing is used to detect 1, 2, 3, 4, 5, 6, 7, 8, 9, 10, 11, 12, or more than 12 of KRT85, TIMP1, FP236383.5, LCE6A, LCE2C, LCE1F, FP236383.4, IL37, LCE5A, AL139247.1, WARS1, and GBP1. In some instances, next generation sequencing is used to detect 1, 2, 3, 4, 5, 6, 7, 8, 9, 10, 11, 12, or more than 12 of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6.

Probes may also be used to detect genes. For example, one or more probes are used to detect at least one of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some cases, no more than 3, 4, 5, 6, 7, 8, 9, 10, 15, 20, or 22 genes are detected. In some instances, a set of probes are used to detect at least two of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least three of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least four of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least five of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least six of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least seven of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least eight of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least nine of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least ten of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least eleven of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect at least twelve of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, a set of probes are used to detect all of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. For example, a set of probes are used to detect at least two of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some cases, no more than 3, 4, 5, 6, 7, 8, 9, 10, 15, 20, or 22 genes are detected. In some instances, one or more probes are used to detect at least one of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least three of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least four of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least five of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least six of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least seven of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least eight of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least nine of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least ten of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least eleven of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect at least twelve of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes are used to detect all of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1.

Probes may also be used to detect genes. For example, one or more probes are used to detect at least one of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some cases, no more than 3, 4, 5, 6, 7, 8, 9, 10, 15, 20, or 22 genes are detected. In some instances, a set of probes are used to detect at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least three of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least four of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least five of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least six of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least seven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least eight of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least nine of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least ten of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least eleven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect at least twelve of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes are used to detect all of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, a set of probes is used to detect 1, 2, 3, 4, 5, 6, 7, 8, 9, 10, 11, 12, or more than 12 of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. In some instances, a set of probes is used to detect 1, 2, 3, 4, 5, or 6 of MT-ND3, JPH3, TIMP2, MT-ND4, SOX11, and MED16. In some instances, a set of probes is used to detect 1, 2, 3, 4, 5, 6, 7, 8, 9, 10, 11, 12, or more than 12 of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, a set of probes is used to detect 1, 2, 3, 4, 5, 6, 7, 8, 9, 10, 11, 12, or more than 12 of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, a set of probes is used to detect 1, 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, a set of probes is used to detect 1, 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, a set of probes is used to detect 1, 2, 3, 4, 5, 6, 7, 8, 9, 10, 11, 12, or more than 12 of KRT85, TIMP1, FP236383.5, LCE6A, LCE2C, LCE1F, FP236383.4, IL37, LCE5A, AL139247.1, WARS1, and GBP1. In some instances, a set of probes is used to detect 1, 2, 3, 4, 5, 6, 7, 8, 9, 10, 11, 12, or more than 12 of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6.

In some embodiments, the one or more probes comprises a polynucleotide. In some instances, the one or more probes comprises polynucleotides for two different exons of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, AND RNA5SP481. In some instances, the gene expression levels are detected following hybridization of one or more probes to at least one of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, the gene expression levels are detected following hybridization of the set of probes to at least two of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some embodiments, the set of probes detects RNA. In some embodiments, the set of probes detects mRNA. In some embodiments, the set of probes detects DNA. In some embodiments, the set of probes comprises polynucleotides. In some instances, the one or more probes comprises a polynucleotide for one or more different exons of MT-ND3, JPH3, TIMP2, MT-ND4, SOX11, MED16, KIF1B, MT-ND1, COL17A, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, the gene expression levels are detected following hybridization of the set of probes to at least two of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some embodiments, the set of probes detects RNA. In some embodiments, the set of probes detects mRNA. In some embodiments, the set of probes detects DNA. In some instances, the set of probes comprises polynucleotides for two different exons of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, the gene expression levels are detected following hybridization of the set of probes to at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, the gene expression levels are detected following hybridization of the set of probes to at least 2, 3, 4, 5, 6, 7, 8, 9, 10, 11, 12, or more than 12 of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP1-1. In some instances, the gene expression levels are detected following hybridization of the set of probes to at least 2, 3, 4, 5, or 6 of MT-ND3, JPH3, TIMP2, MT-ND4, SOX11, and MED16. In some instances, the gene expression levels are detected following hybridization of the set of probes to at least 2, 3, 4, 5, 6, 7, 8, 9, 10, 11, 12, or more than 12 of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1. In some instances, the gene expression levels are detected following hybridization of the set of probes to at least 2, 3, 4, 5, 6, 7, 8, 9, 10, 11, 12, or more than 12 of DSG4, GPRC5D, KRTAP4-11, KRTAP7-1, KRTAP8-1, KRTAP19-5, KRTAP19-1, KRTAP19-3, KRT34, KRTAP-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, the gene expression levels are detected following hybridization of the set of probes to at least 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, the gene expression levels are detected following hybridization of the set of probes to at least 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, the gene expression levels are detected following hybridization of the set of probes to at least 2, 3, 4, 5, 6, 7, 8, 9, 10, 11, 12, or more than 12 of KRT85, TIMP1, FP236383.5, LCE6A, LCE2C, LCE1F, FP236383.4, IL37, LCE5A, AL139247.1, WARS1, and GBP1. In some instances, the gene expression levels are detected following hybridization of the set of probes to at least 2, 3, 4, 5, 6, 7, 8, 9, 10, 11, 12, or more than 12 of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6. In some instances, the gene expression levels are detected following hybridization of the set of probes to at least two exons of at least 2, 3, 4, 5, 6, 7, 8, 9, 10, 11, 12, or more than 12 of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP1-1. In some instances, the gene expression levels are detected following hybridization of the set of probes to at least two exons of at least 2, 3, 4, 5, or 6 of MT-ND3, JPH3, TIMP2, MT-ND4, SOX11, and MED16. In some instances, the gene expression levels are detected following hybridization of the set of probes to at least two exons of at least 2, 3, 4, 5, 6, 7, 8, 9, 10, 11, 12, or more than 12 of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP-1. In some instances, the gene expression levels are detected following hybridization of the set of probes to at least two exons of at least 2, 3, 4, 5, 6, 7, 8, 9, 10, 11, 12, or more than 12 of DSG4, GPRC5D, KRTAP4-11, KRTAP7-1, KRTAP8-1, KRTAP19-5, KRTAP19-1, KRTAP19-3, KRT34, KRTAP-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, the gene expression levels are detected following hybridization of the set of probes to at least two exons of at least 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, the gene expression levels are detected following hybridization of the set of probes to at least two exons of at least 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, the gene expression levels are detected following hybridization of the set of probes to at least two exons of at least 2, 3, 4, 5, 6, 7, 8, 9, 10, 11, 12, or more than 12 of KRT85, TIMP1, FP236383.5, LCE6A, LCE2C, LCE1F, FP236383.4, IL37, LCE5A, AL139247.1, WARS1, and GBP1. In some instances, the gene expression levels are detected following hybridization of the set of probes to at least two exons of at least 2, 3, 4, 5, 6, 7, 8, 9, 10, 11, 12, or more than 12 of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KR T28, RNF5, IGF2BP2, TIMP1, EDN1, and KRT16P6.

Probes for detecting gene expression levels of one or more of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, AND RNA5SP481, in certain embodiments, are used for an amplification reaction. In other embodiments, probes for detecting gene expression levels of at least two of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, AND RNA5SP481, in certain embodiments, are used for an amplification reaction. In some embodiments, the amplification reaction is PCR. In some embodiments, the amplification reaction is quantitative such as qPCR. In some instances, the amplification reaction is paired with reverse transcription (RT-qPCR). In some embodiments, the PCR reaction utilizes a TaqMan™ or a similar quantitative PCR technology. Probes for detecting gene expression levels of at least two of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP9-5, KRTAP2-2, KRTAP11-1, RTAP9-4, KRT33B, KRTAP-5, KRTAP3-3, KRTAP2-1, KRTAP3-1, and KRTAP1-1, in certain embodiments, are used for an amplification reaction. Probes for detecting gene expression levels of at least two of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7, in certain embodiments, are used for an amplification reaction. In some embodiments, the amplification reaction is PCR. In some instances, RT-qPCR is used to detect at least three of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least four of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least five of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least six of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least seven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least eight of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least nine of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least ten of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least eleven of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect at least twelve of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect all of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, RT-qPCR is used to detect 1, 2, 3, 4, 5, 6, 7, 8, 9, 10, 11, 12, or more than 12 of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP1-1. In some instances, RT-qPCR is used to detect 1, 2, 3, 4, 5, or 6 of MT-ND3, JPH3, TIMP2, MT-ND4, SOX11, and MED16. In some instances, RT-qPCR is used to detect 1, 2, 3, 4, 5, 6, 7, 8, 9, 10, 11, 12, or more than 12 of KRT33A, KRTAP8.1, KRTAP19-5, KRTAP2-2, KRTAP11-1, RTAP9-4, KRT33B, KRTAP-5, KRTAP3-3, KRTAP2-1, KRTAP3-1, and KRTAP1-1. In some instances, RT-qPCR is used to detect 1, 2, 3, 4, 5, 6, 7, 8, 9, 10, 11, 12, or more than 12 of DSG4, GPRC5D, KRTAP4-11, KRTAP7-1, KRTAP8-1, KRTAP9-5, KRTAP9-1, KRTAP9-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP-5, KRTAP3-1, KRTAP3-2, KRTAP-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, RT-qPCR is used to detect 1, 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, RT-qPCR is used to detect 1, 2, 3, 4, 5, 6, 7, 8, 9, 10, 11, 12, or more than 12 of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, RT-qPCR is used to detect 1, 2, 3, 4, 5, 6, 7, 8, 9, 10, 11, 12, or more than 12 of KRT85, TIMP1, FP236383.5, LCE6A, LCE2C, LCE1F, FP236383.4, IL37, LCE5A, AL139247.1, WARS1, and GBP1. In some instances, RT-qPCR is used to detect 1, 2, 3, 4, 5, 6, 7, 8, 9, 10, 11, 12, or more than 12 of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6.

In some embodiments, a number of probes in the set of probes is at least or about 1, 2, 3, 5, 6, 7, 8, 9, 10, 11, 12, 13, 14, 15, 16, 17, 18, 19, 20, 25, 30, or more than 30 probes. In some embodiments, the number of probes in the set of probes is about 6 probes. In some embodiments, the number of probes in the set of probes is about 7 probes. In some embodiments, the number of probes in the set of probes is about 8 probes. In some embodiments, the number of probes in the set of probes is about 9 probes. In some embodiments, the number of probes in the set of probes is about 13 probes.

In some embodiments, the set of probes comprises one or more primer pairs. In some embodiments, a number of primer pairs is at least or about 1, 2, 3, 5, 6, 7, 8, 9, 10, 11, 12, 13, 14, 15, 16, 17, 18, 19, 20, 25, 30, or more than 30 primer pairs. In some embodiments, the number of primer pairs is about 6 primer pairs. In some embodiments, the number of primer pairs is about 7 primer pairs. In some embodiments, the number of primer pairs is about 13 primer pairs.

In some embodiments, one or more probes in the set of probes is labeled. In some embodiments, the one or more probe is labeled with a radioactive label, a fluorescent label, an enzyme, a chemiluminescent tag, a colorimetric tag, an affinity tag or other labels or tags that are known in the art.

Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione.

In some embodiments, the fluorescent label is a fluorophore, a fluorescent protein, a fluorescent peptide, quantum dots, a fluorescent dye, a fluorescent material, or variations or combinations thereof.

Exemplary fluorophores include, but are not limited to, Alexa-Fluor dyes (e.g., Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750), APC, Cascade Blue, Cascade Yellow and R-phycoerythrin (PE), DyLight 405, DyLight 488, DyLight 550, DyLight 650, DyLight 680, DyLight 755, DyLight 800, FITC, Pacific Blue, PerCP, Rhodamine, and Texas Red, Cy5, Cy5.5, Cy7.

Examples of fluorescent peptides include GFP (Green Fluorescent Protein) or derivatives of GFP (e.g., EBFP, EBFP2, Azurite, mKalama1, ECFP, Cerulean, CyPet, YFP, Citrine, Venus, and YPet.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, and rhodamine 101 sulfonyl chloride (sold under the tradename of TEXAS RED®). In some embodiments, the cyanine dye is Cy3, Cy3B, Cy3.5, Cy5, Cy5.5, Cy7, IRDYE680, Alexa Fluor 750, IRDye800CW, or ICG.

In some embodiments, the gene expression levels of one or more of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof is measured using PCR. In some embodiments, the gene expression levels of one or more of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1, or a combination thereof is measured using PCR. In some embodiments, the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, or a combination thereof is measured using PCR. In some embodiments, the gene expression levels of one or more of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP-5, KRTAP3-3, KRTAP2-1, KRTAP3-1, and KRTAP1-1, or a combination thereof is measured using PCR. In some embodiments, the gene expression levels of one or more of MT-ND3, JPH3, TIMP2, MT-ND4, SOX11, and MED16, or a combination thereof is measured using PCR. In some embodiments, the gene expression levels of one or more of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1, or a combination thereof is measured using PCR. In some embodiments, the gene expression levels of one or more of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77, or a combination thereof is measured using PCR. In some embodiments, the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1, or a combination thereof is measured using PCR. In some embodiments, the gene expression levels of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1, or a combination thereof is measured using PCR. In some embodiments, the gene expression levels of one or more of KRT85, TIMP1, FP236383.5, LCE6A, LCE2C, LCE1F, FP236383.4, IL37, LCE5A, AL139247.1, WARS1, and GBP1, or a combination thereof is measured using PCR. In some embodiments, the gene expression levels of one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, RT-qPCR, hot start PCR, and Nested PCR.

In some embodiments, the expression levels are measured using qPCR. In some embodiments, the qPCR comprises use of detectable labels or detectably-labeled probes or primers. In some embodiments, the qPCR comprises use of fluorescent dyes or fluorescently-labeled probes or primers. In some embodiments, the fluorescent dye is an intercalating dye. Examples of intercalating dyes include, but are not limited to, intercalating dyes include SYBR green I, SYBR green II, SYBR gold, ethidium bromide, methylene blue, Pyronin Y, DAPI, acridine orange, Blue View, or phycoerythrin. In some embodiments, the qPCR comprises use of more than one fluorescent probe or primer. In some embodiments, the use of more than one fluorescent probes or primers allows for multiplexing. For example, different non-classical variants are hybridized to different fluorescent probes or primers and can be detected in a single qPCR reaction.

Methods and compositions described herein, in some embodiments, further comprise detecting a mutational change in a gene of interest. In some instances, the mutational change is detected in TERT, CDKN2A, TP53, PTCH1, or a combination thereof. In some instances, the mutational change is detected in TERT. In some instances, the mutational change is detected in CDKN2A. In some instances, the mutational change is detected in TP53. In some instances, the mutational change is detected in PTCH1. Exemplary amino acid sequences for TERT, CDKN2A, TP53, and PTCH1 are illustrated in Table 1.

TABLE 1
Mutational changes in genomic DNA

| SEQ | | ACCES- | |
|---|---|---|---|
| ID | GENE | SION | |
| NO | NAME | NUMBER | AMINO ACID SEQUENCE |
| 1 | TP53 | P04637 | MEEPQSDPSVEPPLSQETFSDLW |
| | | | KLLPENNVLSPLPSQAMDDLMLS |
| | | | PDDIEQWFTEDPGPDEAPRMPEA |
| | | | APPVAPAPAAPTPAAPAPAPSWP |
| | | | LSSSVPSQKTYQGSYGFRLGFLH |
| | | | SGTAKSVTCTYSPALNKMFCQLA |
| | | | KTCPVQLWVDSTPPPGTRVRAMA |
| | | | IYKQSQHMTEVVRRCPHHERCSD |
| | | | SDGLAPPQHLIRVEGNLRVEYLD |
| | | | DRNTFRHSVVVPYEPPEVGSDCT |
| | | | TIHYNYMCNSSCMGGMNRRPILT |
| | | | IITLEDSSGNLLGRNSFEVRVCA |
| | | | CPGRDRRTEEENLRKKGEPHHEL |
| | | | PPGSTKRALPNNTSSSPQPKKKP |
| | | | LDGEYFTLQIRGRERFEMFRELN |
| | | | EALELKDAQAGKEPGGSRAHSSH |
| | | | LKSKKGQSTSRHKKLMFKTEGPD |
| | | | SD |
| 2 | TERT | O14746 | MPRAPRCRAVRSLLRSHYREVLP |
| | | | LATFVRRLGPQGWRLVQRGDPAA |
| | | | FRALVAQCLVCVPWDARPPPAAP |
| | | | SFRQVSCLKELVARVLQRLCERG |
| | | | AKNVLAFGFALLDGARGGPPEAF |
| | | | TTSVRSYLPNTVTDALRGSGAWG |
| | | | LLLRRVGDDVLVHLLARCALFVL |
| | | | VAPSCAYQVCGPPLYQLGAATQA |
| | | | RPPPHASGPRRRLGCERAWNHSV |
| | | | REAGVPLGLPAPGARRRGGSASR |
| | | | SLPLPKRPRRGAAPEPERTPVGQ |
| | | | GSWAHPGRTRGPSDRGFCVVSPA |
| | | | RPAEEATSLEGALSGTRHSHPSV |
| | | | GRQHHAGPPSTSRPPRPWDTPCP |
| | | | PVYAETKHFLYSSGDKEQLRPSF |
| | | | LLSSLRPSLTGARRLVETIFLGS |
| | | | RPWMPGTPRRLPRLPQRYWQMRP |
| | | | LFLELLGNHAQCPYGVLLKTHCP |
| | | | LRAAVTPAAGVCAREKPQGSVAA |
| | | | PEEEDTDPRRLVQLLRQHSSPWQ |
| | | | VYGFVRACLRRLVPPGLWGSRHN |
| | | | ERRFLRNTKKFISLGKHAKLSLQ |
| | | | ELTWKMSVRDCAWLRRSPGVGCV |
| | | | PAAEHRLREEILAKFLHWLMSVY |
| | | | VVELLRSFFYVTETTFQKNRLFF |
| | | | YRKSVWSKLQSIGIRQHLKRVQL |
| | | | RELSEAEVRQHREARPALLTSRL |
| | | | RFIPKPDGLRPIVNMDYVVGART |
| | | | FRREKRAERLTSRVKALFSVLNY |
| | | | ERARRPGLLGASVLGLDDIHRAW |
| | | | RTFVLRVRAQDPPPELYFVKVDV |
| | | | TGAYDTIPQDRLTEVIASIIKPQ |
| | | | NTYCVRRYAVVQKAAHGHVRKAF |
| | | | KSHVSTLTDLQPYMRQFVAHLQE |
| | | | TSPLRDAVVIEQSSSLNEASSGL |
| | | | FDVFLRFMCHHAVRIRGKSYVQC |
| | | | QGIPQGSILSTLLCSLCYGDMEN |
| | | | KLFAGIRRDGLLLRLVDDFLLVT |
| | | | PHLTHAKTFLRTLVRGVPEYGCV |
| | | | VNLRKTVVNFPVEDEALGGTAFV |
| | | | QMPAHGLFPWCGLLLDTRTLEVQ |
| | | | SDYSSYARTSIRASLTFNRGFKA |
| | | | GRNMRRKLFGVLRLKCHSLFLDL |
| | | | QVNSLQTVCTNIYKILLLQAYRF |
| | | | HACVLQLPFHQQVWKNPTFFLRV |
| | | | ISDTASLCYSILKAKNAGMSLGA |
| | | | KGAAGPLPSEAVQWLCHQAFLLK |
| | | | LTRHRVTYVPLLGSLRTAQTQLS |
| | | | RKLPGTTLTALEAAANPALPSDF |
| | | | KTILD |
| 3 | PTCH1 | Q13635 | MASAGNAAEPQDRGGGGSGCIGA |
| | (iso- | | PGRPAGGGRRRRTGGLRRAAAPD |
| | form L) | | RDYLHRPSYCDAAFALEQISKGK |
| | | | ATGRKAPLWLRAKFQRLLFKLGC |
| | | | YIQKNCGKFLVVGLLIFGAFAVG |
| | | | LKAANLETNVEELWVEVGGRVSR |
| | | | ELNYTRQKIGEEAMFNPQLMIQT |
| | | | PKEEGANVLTTEALLQHLDSALQ |
| | | | ASRVHVYMYNRQWKLEHLCYKSG |
| | | | ELITETGYMDQIIEYLYPCLIIT |
| | | | PLDCFWEGAKLQSGTAYLLGKPP |
| | | | LRWTNFDPLEFLEELKKINYQVD |
| | | | SWEEMLNKAEVGHGYMDRPCLNP |
| | | | ADPDCPATAPNKNSTKPLDMALV |
| | | | LNGGCHGLSRKYMHWQEELIVGG |
| | | | TVKNSTGKLVSAHALQTMFQLMT |
| | | | PKQMYEHFKGYEYVSHINWNEDK |
| | | | AAAILEAWQRTYVEVVHQSVAQN |
| | | | STQKVLSFTTTTLDDILKSFSDV |
| | | | SVIRVASGYLLMLAYACLTMLRW |
| | | | DCSKSQGAVGLAGVLLVALSVAA |
| | | | GLGLCSLIGISFNAATTQVLPFL |
| | | | ALGVGVDDVFLLAHAFSETGQNK |
| | | | RIPFEDRTGECLKRTGASVALTS |
| | | | ISNVTAFFMAALIPIPALRAFSL |
| | | | QAAVVVVFNFAMVLLIFPAILSM |
| | | | DLYRREDRRLDIFCCFTSPCVSR |
| | | | VIQVEPQAYTDTHDNTRYSPPPP |
| | | | YSSHSFAHETQITMQSTVQLRTE |
| | | | YDPHTHVYYTTAEPRSEISVQPV |
| | | | TVTQDTLSCQSPESTSSTRDLLS |
| | | | QFSDSSLHCLEPPCTKWTLSSFA |
| | | | EKHYAPFLLKPKAKVVVIFLFLG |
| | | | LLGVSLYGTTRVRDGLDLTDIVP |
| | | | RETREYDFIAAQFKYFSFYNMYI |
| | | | VTQKADYPNIQHLLYDLHRSFSN |
| | | | VKYVMLEENKQLPKMWLHYFRDW |
| | | | LQGLQDAFDSDWETGKIMPNNYK |
| | | | NGSDDGVLAYKLLVQTGSRDKPI |
| | | | DISQLTKQRLVDADGIINPSAFY |
| | | | IYLTAWVSNDPVAYAASQANIRP |
| | | | HRPEWVHDKADYMPETRLRIPAA |
| | | | EPIEYAQFPFYLNGLRDTSDFVE |
| | | | AIEKVRTICSNYTSLGLSSYPNG |
| | | | YPFLFWEQYIGLRHWLLLFISVV |
| | | | LACTFLVCAVFLLNPWTAGIIVM |
| | | | VLALMTVELFGMMGLIGIKLSAV |
| | | | PVVILIASVGIGVEFTVHVALAF |
| | | | LTAIGDKNRRAVLALEHMFAPVL |
| | | | DGAVSTLLGVLMLAGSEFDFIVR |
| | | | YFFAVLAILTILGVLNGLVLLPV |
| | | | LLSFFGPYPEVSPANGLNRLPTP |
| | | | SPEPPPSVVRFAMPPGHTHSGSD |
| | | | SSDSEYSSQTTVSGLSEELRHYE |
| | | | AQQGAGGPAHQVIVEATENPVFA |
| | | | HSTVVHPESRHHPPSNPRQQPHL |
| | | | DSGSLPPGRQGQQPRRDPPREGL |
| | | | WPPPYRPRRDAFEISTEGHSGPS |
| | | | NRARWGPRGARSHNPRNPASTAM |
| | | | GSSVPGYCQPITTVTASASVTVA |
| | | | VHPPPVPGPGRNPRGGLCPGYPE |
| | | | TDHGLFEDPHVPFHVRCERRDSK |
| | | | VEVIELQDVECEERPRGSSSN |
| 4 | PTCH1 | NP_0010770 | MFNPQLMIQTPKEEGANVLTTEA |
| | (iso- | 74.1 | LLQHLDSALQASRVHVYMYNRQW |
| | form S) | | KLEHLCYKSGELITETGYMDQII |
| | | | EYLYPCLIITPLDCFWEGAKLQS |
| | | | GTAYLLGKPPLRWTNFDPLEFLE |
| | | | ELKKINYQVDSWEEMLNKAEVGH |
| | | | GYMDRPCLNPADPDCPATAPNKN |
| | | | STKPLDMALVLNGGCHGLSRKYM |
| | | | HWQEELIVGGTVKNSTGKLVSAH |
| | | | ALQTMFQLMTPKQMYEHFKGYEY |
| | | | VSHINWNEDKAAAILEAWQRTYV |
| | | | EVVHQSVAQNSTQKVLSFTTTTL |
| | | | DDILKSFSDVSVIRVASGYLLML |
| | | | AYACLTMLRWDCSKSQGAVGLAG |
| | | | VLLVALSVAAGLGLCSLIGISFN |
| | | | AATTQVLPFLALGVGVDDVFLLA |
| | | | HAFSETGQNKRIPFEDRTGECLK |
| | | | RTGASVALTSISNVTAFFMAALI |
| | | | PIPALRAFSLQAAVVVVFNFAMV |
| | | | LLIFPAILSMDLYRREDRRLDIF |
| | | | CCFTSPCVSRVIQVEPQAYTDTH |
| | | | DNTRYSPPPPYSSHSFAHETQIT |
| | | | MQSTVQLRTEYDPHTHVYYTTAE |
| | | | PRSEISVQPVTVTQDTLSCQSPE |
| | | | STSSTRDLLSQFSDSSLHCLEPP |
| | | | CTKWTLSSFAEKHYAPFLLKPKA |
| | | | KVVVIFLFLGLLGVSLYGTTRVR |
| | | | DGLDLTDIVPRETREYDFIAAQF |
| | | | KYFSFYNMYIVTQKADYPNIQHL |
| | | | LYDLHRSFSNVKYVMLEENKQLP |
| | | | KMWLHYFRDWLQGLQDAFDSDWE |
| | | | TGKIMPNNYKNGSDDGVLAYKLL |
| | | | VQTGSRDKPIDISQLTKQRLVDA |
| | | | DGIINPSAFYIYLTAWVSNDPVA |
| | | | YAASQANIRPHRPEWVHDKADYM |
| | | | PETRLRIPAAEPIEYAQFPFYLN |
| | | | GLRDTSDFVEAIEKVRTICSNYT |
| | | | SLGLSSYPNGYPFLFWEQYIGLR |
| | | | HWLLLFISVVLACTFLVCAVFLL |
| | | | NPWTAGIIVMVLALMTVELFGMM |
| | | | GLIGIKLSAVPVVILIASVGIGV |
| | | | EFTVHVALAFLTAIGDKNRRAVL |
| | | | ALEHMFAPVLDGAVSTLLGVLML |
| | | | AGSEFDFIVRYFFAVLAILTILG |
| | | | VLNGLVLLPVLLSFFGPYPEVSP |
| | | | ANGLNRLPTPSPEPPPSVVRFAM |
| | | | PPGHTHSGSDSSDSEYSSQTTVS |
| | | | GLSEELRHYEAQQGAGGPAHQVI |
| | | | VEATENPVFAHSTVVHPESRHHP |
| | | | PSNPRQQPHLDSGSLPPGRQGQQ |
| | | | PRRDPPREGLWPPPYRPRRDAFE |
| | | | ISTEGHSGPSNRARWGPRGARSH |
| | | | NPRNPASTAMGSSVPGYCQPITT |
| | | | VTASASVTVAVHPPPVPGPGRNP |
| | | | RGGLCPGYPETDHGLFEDPHVPF |
| | | | HVRCERRDSKVEVIELQDVECEE |
| | | | RPRGSSSN |
| 5 | CDKN2A | P42771 | MEPAAGSSMEPSADWLATAAARG |
| | | | RVEEVRALLEAGALPNAPNSYGR |
| | | | RPIQVMMMGSARVAELLLLHGAE |
| | | | PNCADPATLTRPVHDAAREGFLD |
| | | | TLVVLHRAGARLDVRDAWGRLPV |
| | | | DLAEELGHRDVARYLRAAAGGTR |
| | | | GSNHARIDAAEGPSDIPD |

TERT, also known as Telomerase Reverse Transcriptase or Telomerase-Associated Protein 2, encodes the TERT protein. The TERT protein is the catalytic subunit of the protein telomerase. In some instances, a mutation in TERT is correlated with a non-melanoma skin cancer (e.g., BCC and/or SCC). In some instances, a mutation is in the TERT promoter. In some instances, a mutation is at least or about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, or more than 200 base pairs upstream of the translation start site of the TERT promoter. In some instances, one or more mutations are in the TERT promoter. In some instances, the one or more mutations in the TERT promoter are a G to A mutation. In some instances, the one or more mutations in the TERT promoter are a T to G mutation. In some instances, the one or more mutations in the TERT promoter are a C to T mutation. In some instances, one or more mutations in the TERT promoter result in increased expression of TERT. In some instances, one or more mutations in the TERT promoter result in increased expression or activity of TERT protein.

Exemplary mutations in TERT include, but not limited to, 1,295,228 C>T (C228T) and 1,295,250 C>T (C250T). In some instances, C228T is a mutation corresponding to −124 C>T from the translation start site in the TERT promoter. In some instances, C250T is a mutation corresponding to −146 C>T from the translation start site in the TERT promoter. In some instances, a mutation is a nucleotide sequence of TERT. For example, the mutation in the nucleotide sequence includes, but not limited to, 571A>G, 648G>T, 1127C>T, 1135T>C, 1216G>A, 1217G>A, 1281C>T, 1284G>A, 1405C>T, 1461C>T, 1529G>A, 1541T>A, 1566G>A, 1689C>T, 1695G>A, 1782G>A, 1831G>A, 1841C>T, 1882G>A, 1928G>A, 2009C>A, 2067C>T, 2152G>A, 2162C>G, 2163C>T, 2178G>A, 2208G>A, 2254C>A, 2262C>G, 2271G>A, 2272G>A, 2283C>T, 2328C>T, 2361G>A, 2391C>T, 2405G>A, 2436C>T, 2456G>A, 2472C>T, 2499G>A, 2508C>A, 2568G>A, 2589C>T, 2633C>T, 2640G>A, 2725G>A, 2750C>T, 2755T>A, 2758G>A, 2773C>T, 2784C>T, 2786C>T, 2896G>A, 3015C>T, 3057C>T, 3084C>A, 3096C>T, 3097C>T, 3139C>T, 3198C>T, 3200C>T, 3284C>G, 3345G>A, 3363G>A, 1-100C>T, 1-101C>T, 1-101_1-100CC>TT, 1-106_1-105CC>TT, 1-111C>T, 1-124C>A, 1-124C>T, 1-125C>T, 1-125_1-124CC>TT, 1-126C>T, 1-126_1-124CCC>TTT, 1-126_1-125CC>TT, 1-127_1-126CC>TT, 1-139_1-138CC>TT, 1-144C>T, 1-145C>T, 1-146C>T, 1-149C>T, 1-150C>T, 1-154C>T, 1-156C>T, 1-156C>T, 1-159C>T, 1-176C>T, 1-187C>T, 1-242C>T, 1-46C>T, 1-57A>C, 1-58C>T, 1-90_1-89GC>TT, and 1-91C>T.

In some instances, a mutation in TERT is in exon 1, 2, 3, 4, 5, 6, 7, 8, 9, 10, 11, 12, 13, 14, 15, 16, or a combination thereof of TERT. In some instances, a mutation in TERT is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, S191G, L216L, P376L, L379L, G406R, G406E, A427A, R428R, L469L, F487F, W510*, V514E, R522R, V563V, E565E, K594K, E611K, P614L, D628N, R643K, A670E, A689A, D718N, P721R, P721P, T726T, Q736Q, H752N, H754Q, K757K, A758T, S761S, F776F, R787R, S797S, S802N, F812F, R819H, S824S, Q833Q, 18361, G856G, L863L, T878I, A880A, V909I, T917M, F919I, V920I, H925Y, F928F, P929L, A966T, L1005L, L1019L, N1028K, F1032F, L1033L, A1040T, L1047L, P1066P, S1067F, S1095*, L1115L, and P1121P. In some cases, the mutation(s) are at the corresponding residue positions as set forth in SEQ ID NO: 2.

The gene CDKN2A, also known as cyclin-dependent kinase inhibitor 2A, encodes two proteins p16^{INK4a} and p14^{ARF}. p16^{INK4a} is transcribed from exon 1α and p14^{ARF} is transcribed from exon 1β and both genes are involved in cellular senescence. In some instances, a mutation in CDKN2A is correlated with a non-melanoma skin cancer (e.g., BCC and/or SCC). In some instances, a mutation is a nucleotide sequence of CDKN2A. For example, the mutation in the nucleotide sequence includes, but not limited to, 1_471del471, 4G>A, 9_32del24, 12G>T, 12G>A, 42C>G, 44G>A, 45G>A, 47_50delTGGC, 58G>C, 66_67GG>AA, 68delG, 83T>G, 92T>C, 95_112del18, 97G>T, 104G>A, 104G>T, 106delG, 107C>G, 109C>T, 113C>T, 128_129delGT, 132delC, 142_143CC>TT, 143C>T, 144G>A, 147_148CC>AT, 147_148CC>TT, 148C>T, 151_457del307, 158_159delTG, 161T>A, 163G>C, 164G>A, 166_167insA, 168_169insG, 169_170GC>TT, 170C>T, 170_172CCC>TTT, 171C>T, 171_172CC>TT, 171_178delCCGAGTGG, 172C>T, 172_173insC, 172_179delCGAGTGGC, 172delC, 176T>G, 179C>T, 179_180insC, 181G>T, 181_202>AC, 185T>C, 188T>C, 192G>A, 194T>C, 196_198CAC>TAG, 198_199insA, 199G>A, 202G>A, 203_204delCG, 203delC, 204G>A, 205G>T, 209C>T, 212A>T, 215G>A, 218C>T, 219C>T, 222C>A, 223C>T, 223_224CC>T, 225_243del19, 227C>T, 236C>T, 237C>T, 237_238CC>TT, 238C>T, 238_247del10, 239G>A, 241C>T, 242C>A, 242C>T, 242_243CC>TT, 243_244ins19, 243_244insC, 244G>A, 245T>A, 245T>C, 247C>G, 247C>T, 248A>G, 248A>T, 248_249AC>CT, 249C>G, 250G>A, 250_270del21, 254C>T, 257_259delCCC, 259C>T, 259delC, 260G>A, 261_262GG>AA, 262G>A, 262G>T, 264_265GG>AA, 266delG, 268T>C, 270C>T, 286_294del9, 290T>C, 290T>G, 290_294delTGCAC, 295C>T, 297G>A, 297_298GG>AC, 299C>T, 301G>T, 304G>A, 305C>T, 311T>G, 319C>T, 320G>A, 329G>A, 329_330GG>AA, 330G>A, 331G>A, 334C>G, 335G>C, 341C>A, 341C>T, 341_342CC>TT, 342C>T, 346G>T, 346_347insG, 370C>T, 371G>A, 373G>A, 373_374insCG, 377T>A, 380delC, 386_387AC>TT, 387C>G, 389T>G, 406G>A, 413G>A, 442G>A, 443C>G, 457G>A, 470G>C, 1_150del150, 1_457del457, 151-1_151GG>AA, 151_151G>A, 151_457del307, 151_457del321, 458_471del14, (199_204)delG, 150+15T>C, 150+1G>A, 150+2T>C, 150+8G>C, 151-1G>A, 151-1G>T, 151-2A>G, 151-2A>T, 151-42C>T, 457+1G>A, and 458-2A>G.

In some instances, mutations in CDKN2A comprise deletions and/or mutations throughout the coding region. In some instances, a mutation in CDKN2A is in exon 1, 2, 3, 4, 5, 6, 7, 8, or a combination thereof of CDKN2A.

In some instances, a mutation in CDKN2A is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, E2K, P3_P11del, A4S, A4A, D14E, W15*, W15*, L16fs*9, A20P, G23S, G23fs*3, R24P, V28G, L31P, L32_L37del, L32_L37del, E33*, G35E, G35V, A36fs*17, A36G, A36T, L37L, P38L, S43fs*76, Y44fs*1, P48L, P48L, P48P, P48L, I49M, Q50*, Q50*, Q50*, Q50*, V51fs, M53I, M53fs*66, M54K, M54R, G55R, G55D, S56fs*64, A57fs*63, A57F, A57V, A57_R58>V*, A57A, R58*, R58fs*59, R58*, R58fs*62, R58fs*59, R58fs*88, R58*, V59G, A60V, A60fs*, E61fs*59, E61*, E61fs*52, L62P, L63P, L64L, L65P, H66*, G67*, G67fs*53, G67S, A68T, A68fs*51, A68fs*78, A68A, E69*, P70L, N711, C72Y, A73V, A73A, D74E, P75S, P75fs*71, A76fs*64, A76V, T791, T79T, R80*, R80*, R80fs*63, R80Q, R80*, R80, P81S, P81H, P81L, P81L, P81L, V82fs*44, V82fs*38, V82M, V82E, V82A, H83D, H83Y, H83R, H83L, H83P, H83Q, H83N, H83Y, D84N, D84_F90del, A85V, A86_R87>G, R87W, R87fs*59, R87Q, E87K, E88K, E88*, E88*, G89S, G89fs*57, F90L, F90F, V96_H98del, L97P, L97R, L97fs*21, R99W, R99R, A100P, A100V, G101W, G101W, A102T, A102V, L104R, R107C, R107H, A109V, W110*, W110*, W110*, W110*, G111S, R112G, R112P, P114H, P114L, P114L, P114P, P114L, D116Y, D116fs*4, R124C, R124H, D125N, D125fs*22, V126D, V126D, A127fs*19, Y129F, Y129*, L130R, L130R, G136S, R138K, A148T, A148G, D153N, and *157S. In some cases, the mutation(s) are at the corresponding residue positions as set forth in SEQ ID NO: 5.

In some instances, CDKN2A comprises one or more mutations in the protein region QVMMMGSARVAELLLLHGAEPNCADPATLTRPVHDAAREGFLDTL VVLHRAGARLDVRDAW GRLPVDLAEELGHRDVARYLRAAAGGTRGSNHARIDAAEGPS (SEQ ID NO: 6). In some cases, CDKN2A comprises a mutation at V51fs, M53I, R58*, E61*, G67*, E69*, or R80*, or a combination thereof, in which fs denotes frameshift and (*) denotes nonsense substitution.

TP53, also known as p53, cellular tumor antigen p53, phosphoprotein p53, tumor suppressor p53, antigen NY-CO-13, or transformation-related protein 53 (TRP53), encodes the tumor protein p53 (TP53). TP53 is a phosphoprotein made of 393 amino acids and comprises four domains. TP53 plays a role in cell cycle control and apoptosis. In some instances, a mutation in TP53 is associated with a non-melanoma skin cancer (e.g., BCC and/or SCC). In some instances, a mutation is a nucleotide sequence of TP53. For example, the mutation in the nucleotide sequence includes, but not limited to, 96+1G>A, 96+1G>T, 97-1G>A, 375+1G>A, 375+2T>C, 375_375+1GG>AT, 376-1G>A, 376-1G>T, 559+1G>A, 559+2T>G, 560-1G>A, 560-1G>T, 560-1_560GG>AA, 560-2A>C, 672+1G>A, 673-1G>A, 673-8T>A, 782+1G>C, 782_782+1GG>AA, 783-2A>T, 919+1G>A, 920-1G>T, 993+1G>A, 994-1G>A, 19G>C, 31G>A, 37C>T, 69G>A, 79C>T, 101C>T, 102C>G, 136T>C, 139_140delCC, 140delC, 142G>A, 151G>T, 158G>A, 159G>A, 159G>C, 162C>T, 166G>T, 173delC, 175_176GG>AA, 181G>A, 202G>A, 206C>T, 211C>T, 212C>T, 214C>G, 214C>T, 215C>G, 216_217insC, 217G>A, 229C>T, 238C>T, 239C>T, 242C>T, 242delC, 245C>T, 248C>A, 250G>A, 250_251insT, 251C>T, 251_252CC>TT, 253C>T, 254C>T, 257_279del23, 265C>T, 265_266CC>TT, 266C>T, 269C>T, 272G>A, 273G>A, 275C>T, 281C>T, 284C>T, 287C>T, 289G>A, 292C>T, 293C>T, 296C>T, 298C>T, 305C>T, 309C>G, 310C>T, 312delG, 313delG, 321C>A, 321C>G, 322G>A, 325_330delTTCCGT, 326T>C, 327_328CC>TT, 328C>T, 328delC, 332T>A, 349delG, 358A>G, 365_367delTGA, 375G>A, 375G>T, 380C>T, 380_381CC>TT, 382C>T, 386C>T, 388C>T, 388delC, 394A>C, 394A>G, 395A>T, 396G>T, 398T>A, 400T>C, 403T>C, 404G>A, 404G>C, 405C>A, 405_406CC>TT, 406C>T, 409C>A, 412delG, 413C>T, 413_414CC>TT, 415A>T, 416delA, 417G>C, 418_419insN, 419C>T, 424C>T, 424_425CC>TT, 425C>T, 428T>A, 428T>G, 430delC, 432G>A, 434_435TG>GT, 437G>A, 438G>A, 442_465del24, 446C>T, 447C>T, 449C>T, 451C>T, 452C>A, 452_453CC>TT, 453C>T, 453_454insN, 454C>T, 454_455CC>TT, 455C>G, 455C>T, 457_461delCCCGG, 456_457insC, 459_460insN, 463_464delAC, 465C>T, 466C>T, 466delC, 467G>C, 468_469delCG, 468_487del20, 469G>A, 469G>T, 471_472CC>TT, 472C>T, 474C>T, 475G>A, 476C>T, 476_477CC>TT, 477C>T, 480G>A, 480G>C, 481G>A, 482C>T, 483C>T, 487T>C, 487T>G, 493C>T, 496T>G, 502C>T, 502_503insN, 502_511del10, 507G>A, 508A>G, 509C>T, 511G>C, 513delG, 517G>A, 517G>C, 518T>G, 521G>A, 522G>C, 524G>A, 527G>A, 527G>T, 528C>G, 529C>T, 529_530CC>TT, 530C>T, 530_531CC>AT, 530_531CC>TT, 528delC, 531_532CC>TT, 532C>A, 532C>T, 532_533insN, 534_535CC>AT, 534_535CC>TT, 535C>A, 535C>T, 536A>T, 541C>T, 542G>A, 546C>T, 548C>A, 548C>T, 550G>A, 556G>A, 559G>A, 565G>A, 565_591del27, 566C>T, 567C>T, 568C>T, 568_569CC>TT, 569C>T, 571C>T, 572C>T, 573T>A, 574C>T, 580C>T, 581T>G, 582_586delTATCC, 583A>T, 585_586CC>TT, 586C>T, 587G>T, 590T>A, 592G>A, 592G>T, 599delA, 600_601insN, 601T>G, 603_604GC>TT, 603_604insAAATTTG, 605G>C, 605G>T, 605_606GT>CG, 606delT, 613T>G, 614A>G, 617T>A, 620_627delATGACAGA, 622G>T, 626G>A, 626_627delGA, 632C>T, 637C>T, 638G>A, 638G>C, 640_647delCATAGTGT, 645T>G, 647T>G, 652G>A, 653T>A, 653T>G, 656C>T, 656_657CC>TT, 658T>A, 659A>G, 660_661insN, 662_672+40del51, 664_665CC>TT, 665C>T, 666G>C, 667delC, 670G>T, 674T>C, 677G>A, 677G>C, 680C>T, 682G>A, 683A>C, 685_686delTG, 688A>G, 689C>T, 690C>A, 691A>T, 697delC, 700T>A, 700T>C, 701A>C, 702C>A, 703A>G, 704A>G, 704A>T, 706T>A, 706T>C, 712T>A, 713G>A, 713G>T, 714T>G, 721T>C, 722C>T, 722_723CC>TT, 723C>T, 724T>A, 724T>G, 725G>A, 726C>T, 727A>T, 728T>G, 730G>A, 733G>A, 733G>T, 733_734GG>AA, 734G>A, 734G>T, 737T>C, 737T>G, 738G>A, 739A>T, 740A>C, 741_742CC>TT, 742C>T, 742_744CGG>TGC, 743G>A, 743G>C, 743G>T, 743_744GG>AA, 744G>A, 745A>T, 745_768del24, 746G>C, 746G>T, 747G>C, 747G>T, 748C>G, 748C>T, 748_749CC>TT, 749C>T, 749_750CC>TG, 750C>T, 752T>A, 755T>C, 756C>T, 757A>G, 758C>T, 759C>T, 762delC, 770T>C, 771_772GG>AA, 772G>A, 775delG, 776A>T, 781A>T, 791delT, 794T>C, 795G>A, 795_796GG>AA, 796G>A, 796G>T, 796_797GG>AA, 797G>A, 799C>T, 800G>C, 806G>A, 808T>A, 811G>A, 812_815delAGGT, 814G>A, 815T>A, 815T>G, 817C>G, 817C>T, 817_825delCGTGTTTG . . . , 818G>A, 818G>T, 820G>A, 821T>C, 824G>A, 824G>C, 825T>G, 826G>C, 827C>A, 827C>T, 827_829CCT>TC, 829T>G, 830G>A, 830G>T, 832C>A, 832C>T, 832_833CC>TT, 833C>G, 833C>T, 834_835insN, 835G>A, 836G>A, 836_837GG>AA, 837G>A, 838A>T, 839G>A, 839G>C, 841G>A, 843C>A, 843C>G, 843C>T, 843_844CC>AT, 843_844CC>TT, 844C>G, 844C>T, 845G>A, 845G>T, 847C>T, 852A>T, 853G>A, 853G>C, 854A>T, 855G>A, 855_856GG>AA, 856G>A, 856G>C, 856G>T, 857A>T, 859G>A, 863delA, 865C>T, 867C>T, 868C>T, 868delC, 869G>A, 870C>G, 880G>A, 880G>T, 882G>A, 884C>T, 888_889CC>TT, 890delA, 892G>T, 898C>T, 899C>G, 898delC, 901C>T, 902_903insC, 904delG, 908G>C, 919G>T, 947C>T, 948_949CC>TT, 949C>T, 955A>G, 960G>A, 965C>T, 968T>C, 972T>A, 976G>T, 981T>A, 986_987CC>TT, 987C>A, 989T>G, 991C>T, 992_993insN, 1006G>T, 1009C>T, 1014C>T, 1023_1024CC>TT, 1024C>T, 1045G>T, 1050delC, 1051A>G, 1072G>A, 1082G>A, 1083delG, 1084delA, 1133C>T, 1143A>T, 559+11G>T, 559+37T>G, 74+12C>T, 783-57A>G, and 919+40delG.

In some instances, mutations in TP53 comprise deletions and mutations throughout the coding region. In some instances, a mutation in TP53 is in exon 1, 2, 3, 4, 5, 6, 7, 8, 9, 10, 11, or a combination thereof of TP53. In some instances, a mutation in TP53 is in exon 5, 7, 8, or a combination thereof of TP53. In some instances, a mutation in TP53 is in exon 5. In some instances, a mutation in TP53 is in exon 7. In some instances, a mutation in TP53 is in exon 8.

In some instances, a mutation in TP53 is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, D7H, E11K, P13S, W23*, P27S, V31I, P34L, P34P, S46P, S46F, P47fs*4, P47fs*76, D48N, E51*, W53*, W53*, W53C, F54F, E56*, P58fs*65, G59N, D61N, E68K, A69V, P71S, P71L, P72A, P72S, P72R, V73fs*76, V73M, P77S, P77del, P80S, P80L, T81I, T81fs*42, P82L, A83E, A84T, A84fs*65, A84V, A84V, P85S, P85L, A86fs*55, P87S, P89S, P89F, P89L, S90F, W91*, W91*, P92L, S94L, S95F, S96F, V971, P98S, P98L, P98L, S99F, Q100*, T1021, Y103*, Q104*, Q104*, G105fs*18, G105fs*18, Y107*, Y107*, G108S, F109_R110delFR, F109S, R110C, R110C, R110fs*13, L111Q, T118fs*5, K120E, V122_T123>A, T125T, T125T, Y126*, S127F, S127F, S127F, P128S, A129V, L130F, L130fs*40, N131K, K132Q, K132E, K132M, K132N, M133K, F134L, C135R, C135Y, C135S, C135*, Q136*, Q136*, L137M, A138fs*32, A138V, A138V, K139*, K139fs*31, K139N, T140fs*9, T140I, P142S, P142F, P142L, V143E, V143G, Q144fs*26, Q144Q, L145R, W146*, W146*, V147A, D148_T155delDSTPP . . . , S149F, S149S, T1501, P151S, P151H, P151L, P151P, P152fs*29, P152S, P152L, P152R, P152L, P152L, P152S, P153fs*26, G154fs*27, G154fs*27, T155fs*25, T155T, R156C, R156fs*14, R156P, V157fs*23, V157fs*17, V157I, V157F, R158C, R158C, R158R, A159T, A159V, A159V, A159A, M160I, M160I, A161T, A161V, A161A, Y163H, Y163D, Q165*, S166A, H168Y, H168fs*13, H168fs*3, M169I, T170A, T170M, E171Q, V172fs*2, V173M, V173L, V173G, V173L, R174K, R174S, R175 (e.g., R175H), C176Y, C176F, C176W, P177S, P177F, P177L, P177H, P177L, P177S, H178fs*69, H178Y, H178N, H178Y, H178fs*3, H178_H179>QY, H179Y, H179N, H179Y, H179L, H179Y, R181C, R181H, C182C, S183*, S183L, D184N, D186N, G187S, G187D, A189T, A189_V197delAPPQHL, A189V, A189A, P190S, P190F, P190L, P191S, P191L, P191P, Q192*, L194F, L194R, I195fs*12, I195F, R196*, R196*, R196L, R196*, V197E, E198K, E198*, N200fs*47, L201fs*8, L201V, L201_R202>FC, R202fs*9, R202P, R202L, R202P, V203fs*44, Y205D, Y205C, L206*, D207fs*6, D208Y, R209K, R209fs*6, T2111, R213*, R213Q, R213P, H214fs*5, S215R, V216G, V216G, V216M, V218M, V218E, V218G, V218G, P219L, P219L, P219L, Y220N, Y220C, E221fs*4, E221fs*4, P222L, P222L, P222P, P223fs*24, E224*, V225A, G226D, G226A, S227F, D228N, D228A, C229fs*10, T230A, T230I, T230T, T231S, H233fs*14, Y234N, Y234H, Y234S, Y234*, Y234S, N235D, N235S, N235I, Y236N, Y236H, C238S, C238Y, C238F, C238W, 5240, S241P, S241F, S241F, S241S, C242S, C242G, C242Y, C242C, M243L, M243R, G244S, G245 (e.g., G245S, G245C, G245N, G245D, G245V, G245D, G245R), M246T, M246R, M2461, N247Y, N247T, R248 (e.g., R248W, R248W, R248C, R248Q, R248P, R248L, R248Q, R248R, R248Q, R248W, R248Y), R249 (e.g., R249W, R249_T256delRPILTI . . . , R249T, R249M, R249S, R249S), P250A, P250S, P250F, P250L, P250L, P250P, 1251N, L252P, L252L, T253A, T2531, T253T, 1255fs*90, L257P, E258K, E258K, D259fs*86, D259V, D259Y, S261C, L264fs*81, L265P, L265L, G266R, G266R, G266*, G266K, G266E, R267W, R267P, S269N, F270I, E271K, E271fs*73, V272M, V272E, V272G, R273 (e.g., R273G, R273C, R273_C275delRVC, R273H, R273L, R273C, R273H), V274I, V274A, C275Y, C275S, C275W, A276P, A276D, A276V, A276fs*69, C277G, C277Y, C277F, P278T, P278S, P278F, P278R, P278L, P278F, P278S, G279fs*27, G279R, G279E, G279E, G279G, G279W, R280*, R280K, R280T, D281N, D281E, D281E, D281D, D281_R282>EW, R282 (e.g., R282W, R282G, R282W, R282Q, R282L), R283C, T284 (e.g., T284T), E285K, E285Q, E285V, E285E, E286K, E286K, E286Q, E286*, E286V, E287K, N288fs*57, L289F, L289L, R290C, R290fs*55, R290H, R290R, E294K, E294*, E294E, P295L, H297Y, H297fs*48, E298*, P300S, P300R, P301fs*44, P301S, G302fs*4, S303fs*42, S303T, A307S, P316L, Q317*, Q317*, Q317*, K319E, K320K, P322L, L323P, D324E, E326*, Y327*, T329I, T329T, L330R, Q331*, Q331fs*6, E336*, R337C, F338F, R342*, R342*, E349*, K351fs*19, K351E, E358K, G361E, S362fs*8, S362fs*8, T377P, S378F, and K381N. In some instances, a mutation is R175, 5240, G245, R248, R249, R273, R282, T284, or combinations thereof. In some cases, the mutation(s) are at the corresponding residue positions as set forth in SEQ ID NO: 1.

PTCH1, also known as Patched 1 or Protein Patched Homolog 1, is a gene that encodes PTCH1, a member of the patched family of proteins. PTCH1 is involved in hedgehog signaling pathway. In some instances, mutations in PTCH1 are involved in a non-melanoma skin cancer (e.g., BCC and/or SCC). In some instances, a mutation is a nucleotide sequence of PTCH1. For example, the mutation in the nucleotide sequence includes, but not limited to, 394+1delG, 584_584+1GG>AA, 747-1G>A, 1067+1G>A, 1068-2A>T, 1068-2_1068-1AG>CT, 1216-4_1227del16, 1216-6C>T, 1347+1G>A, 1504-8T>C, 1603-1G>A, 1729-1G>T, 1847+3A>T, 1848-1G>A, 2251-1G>A, 2561-1G>A, 3168+5G>T, 3306+1G>A, 3449+1G>A, 3549+5G>A, 204G>A, 250C>A, 250C>T, 262_274del13, 271G>A, 272G>A, 277_278insA, 286A>T, 290_291insT, 292_310del19, 304T>A, 343_344GG>AA, 378delG, 387G>A, 394G>A, 404G>A, 426T>A, 430_431ins11, 441_442TG>AT, 445G>T, 451G>A, 463C>T, 475A>T, 478C>T, 493G>T, 523C>T, 528_529AC>CT, 549_550CC>TT, 550C>T, 584G>A, 584G>T, 631A>G, 652C>T, 654G>A, 666T>A, 681G>A, 707G>A, 708G>A, 708_709GG>AA, 709G>A, 712_713insA, 713G>A, 717G>C, 724C>T, 751_760del10, 754C>T, 757C>A, 757C>T, 758_776del19, 767G>A, 768G>A, 804_807delAAAG, 809_818del10, 813_819delAAACTAT, 833G>A, 834G>A, 838G>A, 851_872del22, 857_861AGGTT>G, 862G>A, 863G>A, 865delC, 864_871delTCATGGTT, 879_880CC>TT, 992C>T, 994A>T, 1031G>A, 1047delC, 1055G>A, 1062_1063insC, 1062_1063insT, 1082A>C, 1085C>T, 1092_1093CC>TT, 1093C>T, 1106_1107CC>TT, 1108_1111delAAGC, 1138G>T, 1160G>A, 1161G>A, 1167_1168GG>AT, 1196G>A, 1229G>A, 1249C>T, 1249_1250ins28, 1285delG, 1292T>A, 1316T>C, 1324delG, 1356T>G, 1361_1389del29, 1393_1394insC, 1396C>T, 1433C>T, 1434_1437delACTG, 1439C>G, 1450G>A, 1481C>T, 1481_1485delCCTTT, 1510C>T, 1511C>A, 1511C>T, 1557C>T, 1585A>T, 1594C>T, 1595C>T, 1615G>T, 1634G>A, 1667delT, 1673_1695del23, 1688C>T, 1703C>T, 1703_1704CC>TT, 1703_1711delCCGCT . . . , 1717T>A, 1719delC, 1721_1722CC>TT, 1722C>T, 1725C>T, 1726C>T, 1777_1778CC>TT, 1778C>T, 1796_1799delATTT, 1800A>T, 1804C>T, 1847G>A, 1847G>C, 1854C>A, 1863_1864delAG, 1887delC, 1893_1894insC, 1922C>T, 1930C>T, 1959_1969del11, 1977G>A, 1980C>T, 1986_1987CC>TT, 1992C>T, 1993C>G, 1993C>T, 2004C>T, 2008C>T, 2011C>T, 2020delG, 2033C>T, 2038G>T, 2042C>T, 2048C>T, 2050G>T, 2062C>T, 2066C>T, 2072C>T, 2105C>T, 2107G>T, 2120C>T, 2126G>A, 2128G>A, 2128delG, 2134_2144del11, 2146delT, 2147_2148CC>TT, 2178_2179insC, 2207C>T, 2209G>A, 2209G>T, 2265C>T, 2287G>A, 2287delG, 2307_2308CC>TT, 2308C>T, 2321G>A, 2334G>A, 2345C>T, 2364T>A, 2372T>C, 2380C>T, 2385_2399del15, 2397_2418del22, 2400C>T, 2421C>T, 2421_2422CC>TT, 2438C>A, 2439delG, 2446C>T, 2477delT, 2485G>A, 2492_2493insAGTA, 2557C>T, 2566_2568CAG>T, 2588G>A, 2589G>A, 2666A>G, 2693A>G, 2708_2709insAT, 2709_2710insAA, 2713C>T, 2716_2729del14, 2747_2748CC>AT, 2758_2771del14, 2765_2766ins14, 2777G>A, 2777_2778GG>AA, 2778G>A, 2778_2779GG>AA, 2791_2793CCC>T, 2793_2794ins22, 2794_2795insC, 2810C>T, 2812C>T, 2843G>A, 2847C>T, 2865C>A, 2866_2867delAT, 2873delA, 2885G>C, 2891_2892ins17, 2910delG, 2965G>T, 2974G>T, 2985G>T, 3027C>T, 3046C>T, 3054G>A, 3054_3055GG>AA, 3072C>T, 3120C>T, 3138C>T, 3148C>T, 3152_3153GG>AA, 3153G>A, 3196G>T, 3209T>G, 3236G>T, 3240C>T, 3249delG, 3261C>T, 3320_3321CC>TT, 3340A>T, 3356T>A, 3374_3375CC>TT, 3378_3379CC>TT, 3389C>T, 3401T>A, 3422C>T, 3425G>A, 3435C>T, 3487G>A, 3499G>A, 3499G>T, 3509_3538>GGA, 3514C>T, 3583A>T, 3584C>T, 3586C>T, 3590C>T, 3591C>T, 3592C>T, 3603C>T, 3605C>T, 3634G>A, 3641C>T, 3662C>T, 3708_3709GG>AA, 3715C>T, 3724G>A, 3739G>A, 3748C>T, 3815_3816CC>TT, 3833C>T, 3844C>T, 3856_3867del12, 3857C>T, 3859C>T, 3883C>T, 3906C>T, 3917C>T, 3918C>T, 3944T>C, 3970G>A, 4058C>T, 4140C>T, 4179C>T, 4187G>A, 4204C>T, 4205C>T, 4235C>T, 4249C>T, 4324C>T, 4328G>T, 1405_1406ins, 1728_1728+1delGG, 3169-1_3169GG>AA, 1503+3A>T, 1729-2A>T, 2250+25T>C, 3169-2A>G, 3450-1G>A, 3450-2A>T, 3550-27C>T, 394+1G>A, 584+5G>A, 654+1G>A, 654+2T>A, and 945+5G>C.

In some instances, mutations in PTCH1 comprise deletions and mutations throughout the coding region. In some instances, a mutation in PTCH1 is in exon 1, 2, 3, 4, 5, 6, 7, 8, 9, 10, 11, 12, 13, 14, 15, 16, 17, 18, 19, 20, 21, 22, 23, 24, or a combination thereof of PTCH1. In some instances, a mutation in PTCH1 is in exon 2, 3, 5, 6, 8, 10, 12, 14, 15, 17, 18, 22, 23, or a combination thereof of PTCH1. In some instances, a mutation in PTCH1 is in exon 14, 15, 17, or a combination thereof of PTCH1. In some instances, a mutation in PTCH1 is in exon 2, 3, 5, 6, 8, 10, 12, 18, 22, 23, or a combination thereof of PTCH1. In some instances, a mutation in PTCH1 is in exon 14, 15, 17, or a combination thereof and further in combination with one or more from exon 2, 3, 5, 6, 8, 10, 12, 18, 22, or 23. In some instances, a mutation in PTCH1 is in exon 2 of PTCH1. In some instances, a mutation in PTCH1 is in exon 3 of PTCH1. In some instances, a mutation in PTCH1 is in exon 5 of PTCH1. In some instances, a mutation in PTCH1 is in exon 6 of PTCH1. In some instances, a mutation in PTCH1 is in exon 8 of PTCH1. In some instances, a mutation in PTCH1 is in exon 10 of PTCH1. In some instances, a mutation in PTCH1 is in exon 12 of PTCH1. In some instances, a mutation in PTCH1 is in exon 14 of PTCH1. In some instances, a mutation in PTCH1 is in exon 15 of PTCH1. In some instances, a mutation in PTCH1 is in exon 17 of PTCH1. In some instances, a mutation in PTCH1 is in exon 18 of PTCH1. In some instances, a mutation in PTCH1 is in exon 22 of PTCH1. In some instances, a mutation in PTCH1 is in exon 23 of PTCH1.

In some instances, a mutation in PTCH1 is a mutation in a peptide sequence. In some instances, the mutation results in a missense substitution, a nonsense substitution (*), a coding silent substitution, deletion (del), an insertion (ins), or a frameshift (fs). Exemplary mutations include, but are not limited to, G68G, Q84K, Q84*, F88fs*25, G91S, G91D, Y93fs*1, K96*, C98fs*42, C98fs*13, L102M, G115K, E127fs*10, W129*, V132I, R135Q, Y142*, R144fs*19, G148*, E149*, A151T, P155S, I159L, Q160*, E165*, L175F, Q177*, Q184*, Q184*, R195K, R195M, T211A, Q218*, Q218Q, Y222*, L227L, W236*, W236*, W236_E237>*, E237K, G238fs*14, G238E, A239A, Q242*, K251fs*15, P252S, P253T, P253S, L254fs*9, W256*, W256*, K270fs*1, K270fs*10, N272fs*9, W278*, W278*, E280K, K284fs*33, E286fs*37, G288S, G288D, H289fs*35, Y291fs*25, R294C, S331F, R332*, G344D, N349fs*18, G352E, V355fs*82, V355fs*82, Q361P, T362I, Q365*, Q365*, P369L, K370fs*61, E380*, H384fs*, W387*, W387*, D390Y, W399*, S410N, Q417*, Q417fs*29, D429fs*3, L431Q, V439A, V442fs*14, Y452*, C454fs*1, Q466fs*31, Q466*, V469M, A478V, L479fs*11, S480*, A483G, G484R, S494F, S494fs*1, P504S, P504Q, P504L, A519A, K529*, P532S, P532L, E539*, G545E, V556fs*9, F559fs*60, A563V, P568L, P568L, P568_L570delPAL, F5731, S574fs*6, S574F, S574S, L575L, Q576*, P593F, P593L, P593L, L600fs*22, L600F, R602*, S616N, S616T, C618*, R621fs*5, Q628*, Y630fs*63, D632fs*22, P641L, P644S, E653fs*24, Q659Q, S660S, Q663*, L664L, R665G, R665C, E667*, Y668Y, P670S, H671Y, V674fs*19, T678I, E680*, P681L, S683F, E684*, Q688*, P689L, T6911, P702L, E703*, S707F, R709K, D710N, D710fs*36, S713fs*21, S716fs*30, S716F, C727fs*11, A736V, E737K, E737*, A741V, F755F, V763I, V763fs*9, R770*, R770*, G774E, T778T, P782L, Y788*, 1791T, Q794*, K796_F800del, F800fs*23, F800F, T807T, Q808*, P813Q, N814fs*16, Q816*, F826fs*4, V829M, Y831*, Q853*, Q853*, Q856fs*1, W863*, W863*, Q889R, D898G, K904fs*21, Q905fs*20, Q905*, L907fs*4, P916H, Y920fs*34, Y922fs*1, W926*, W926*, W926*, W926_V927>*, W926*, P931fs*27, V932fs*34, V932fs*27, S937F, Q938*, W948*, V949V, Y955*, M956fs*2, T959fs*3, R962T, A965fs*36, E970fs*25, L981F, E989*, E992*, R995S, Y1009Y, Y1013*, L1016F, W1018*, W1018_E1019>*, L1024L, F1040F, F1046F, P1050S, W1051*, W1051*, E1066*, M1070R, S1079I, A1080A, V1084fs*3, I1087I, A1107V, R1114W, L1119Q, P1125L, (=), A1130V, L1134Q, A1141V, G1142E, F1145F, G1163S, G1167R, G1167W, L1170_P1180>WT, P1172S, T1195S, T1195I, P1196S, S1197F, S1197S, P1198S, P1201P, P1202L, G1212S, T1214M, S1221F, E1237K, R1239W, E1242K, E1242K, A1247T, P1250S, P1272L, P1278L, P1282S, P1286_D1289del, P1286L, H1287Y, P1295S, P1302P, P1306L, P1306P, L1315P, P1315L, E1324K, A1353V, A1380A, P1387S, P1393P, G1396E, P1402S, P1402L, P1412L, H1417Y, R1442W, and G1443V. In some instances, a mutation is S616N, S616T, C618*, R621fs*5, Y630fs*63, D632fs*22, P641L, P644S, E653fs*24, Q659Q, Q663*, L664L, R665G, Y668Y, P670S, H671Y, T678I, E680*, P681L, S683F, E684*, Q688*, P689L, T6911, P702L, S707F, S716fs*30, C727fs*11, A736V, or combinations thereof. In some cases, the mutation(s) are at the corresponding residue positions as set forth in SEQ ID NO: 3 or 4.

Expression level or mutational change once detected, in certain embodiments, provides information regarding a disease in an individual. In some instances, expression level of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, AND RNA5SP481, or combinations thereof provides information regarding the disease in the individual. In some instances, expression level of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1, or combinations thereof provides information regarding the disease in the individual. In some instances, expression level of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7, or combinations thereof provides information regarding the disease in the individual. In some instances, expression level of MT-ND3, JPH3, TIMP2, MT-ND4, SOX11, MED16, KRT33A, KRTAP8-1, KRTAP9-5, KRTAP2-2, KRTAP11-1, KRTAP9-4, KRT33B, KRTAP1-5, KRTAP3-3, KRTAP2-1, KRTAP3-1, and KRTAP1-1, or combinations thereof provides information regarding the disease in the individual. In some instances, expression level of MT-ND3, JPH3, TIMP2, MT-ND4, SOX11, and MED16, or combinations thereof provides information regarding the disease in the individual. In some instances, expression level of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1, or combinations thereof provides information regarding the disease in the individual. In some instances, expression level of DSG4, GPRC5D, KRTAP4-11, KRTAP7-1, KRTAP8-1, KRTAP19-5, KRTAP19-1, KRTAP19-3, KRT34, KRTAP-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77, or combinations thereof provides information regarding the disease in the individual. In some instances, expression level of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1, or combinations thereof provides information regarding the disease in the individual. In some instances, expression level of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1, or combinations thereof provides information regarding the disease in the individual. In some instances, expression level of KRT85, TIMP1, FP236383.5, LCE6A, LCE2C, LCE1F, FP236383.4, IL37, LCE5A, AL139247.1, WARS1, and GBP1, or combinations thereof provides information regarding the disease in the individual. In some instances, expression level of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 or combinations thereof provides information regarding the disease in the individual.

In some instances, mutational change of TERT, CDKN2A, TP53, PTCH1, or combinations thereof provides information regarding the disease of the individual. In some instances, both expression level and mutational change provide information regarding the disease in the individual. Information regarding the disease includes, but is not limited to, identification of a disease state, likelihood of treatment success for a given disease state, identification of progression of a disease state, and identification of a disease stage. In some instances, at least one of expression level and mutational change are compared to a control sample for identification of the disease state, determining likelihood of treatment success for the given disease state, identification of progression of the disease state, or identification of the disease stage. In some instances, the control sample is any sample that is used for making any one of these determinations. In some instances, the control sample is from a healthy individual. In some instances, the control is a sample from an individual with a known disease or disorder. In some instances, the control is from a database or reference. In some instances, the control is a normal sample from the same individual. In some instances, the normal sample is a sample that does not comprise cancer, disease, or disorder, or a sample that would test negative for cancer, disease, or disorder. In some instances, the normal sample is assayed at the same time or at a different time.

In some instances, an expression level of one or more genes of interest from a biological sample varies as compared to a control sample. In some instances, the control sample is a non-cancer sample. In some instances, the expression level is of at least two genes selected from a group consisting of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, the control sample is a non-cancer sample. In some instances, the expression level is of at least two genes selected from a group consisting of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, the expression level is of at least two genes selected from a group consisting of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, and KRT7. In some instances, the expression level is of at least two genes selected from a group consisting of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. In some instances, the expression level is of at least two genes selected from a group consisting of MT-ND3, JPH3, TIMP2, MT-ND4, SOX11, and MED16. In some instances, the expression level is of at least two genes selected from a group consisting of KRT33A, KRTAP8.1, KRTAP9-5, KRTAP2-2, KRTAP1-1, RTAP9-4, KRT33B, KRTAP1-5, KRTAP3-3, KRTAP2-1, KRTAP3-1, and KRTAP1-1. In some instances, the expression level is of at least two genes selected from a group consisting of DSG4, GPRC5D, KRTAP4-11, KRTAP7-1, KRTAP8-1, KRTAP9-5, KRTAP9-1, KRTAP9-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, the expression level is of at least two genes selected from a group consisting of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, and GBP1. In some instances, the expression level is of at least two genes selected from a group consisting of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, the expression level is of at least two genes selected from a group consisting of KRT85, TIMP1, FP236383.5, LCE6A, LCE2C, LCE1F, FP236383.4, IL37, LCE5A, AL139247.1, WARS1, and GBP1. In some instances, the expression level is of at least two genes selected from a group consisting of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6.

Expression levels may be compared to a control (sample). In some instances a control comprises healthy skin from an unaffected area of same individual or a different individual. In some instances, the expression level of a gene described herein is at least or about 5%, 6%, 7%, 8%, 9%, 10%, 11%, 12%, 13%, 14%, 15%, 16%, 17%, 18%, 19%, 20%, 22%, 24%, 28%, 30%, 35%, 40%, 45%, 50%, 55%, 60%, 65%, 70%, 75%, 80%, 85%, 90%, 95% or more than 95% increased as compared to control. In some instances, the expression level is increased in a range of about 1% to about 100%, about 10% to about 90%, about 20% to about 80%, about 30% to about 70%, or about 40% to about 60%. In some instances, the expression level is increased at least 1.1, 1.2, 1.5, 1.7, 2, 3, 4, 5, 6, or at least 10 fold increase relative to a control. In some instances, the control sample is any sample that is used for making any one of these determinations. In some instances, the control sample is from a healthy individual. In some instances, the control is a sample from an individual with a known disease or disorder. In some instances, the control is from a database or reference. In some instances, the control is a normal sample from the same individual. In some instances, the normal sample is a sample that does not comprise cancer, disease, or disorder, or a sample that would test negative for cancer, disease, or disorder. In some instances, the normal sample is assayed at the same time or at a different time. In some instances a control comprises a sample having a skin disease that is not SCC. In some instances a control comprises a sample having a skin disease that is not BCC. In some instances a control comprises a sample having AK. A control may be used to differentiate between skin diseases, such as BCC and SCC. In some instances a control comprises a sample having SCC or BCC. Differences between expression levels of two or more gene in some instances are expressed with a statistical measurement used to validate a hypothesis against observed data. In some instances, the hypothesis comprises whether the sample or patient has a skin disorder. In some instances, the statistical measurement comprises a p-value. In some instances, a diagnosis for a disease or condition described herein (e.g., BCC, SCC, or other disease) is made based on one or more genes having a p-value. In some instances, the p-value is no more than 0.5, 0.4, 0.3, 0.2, 0.15, 0.12, 0.1, 0.9, 0.8, 0.5, 0.4, 0.3, 0.2, 0.1, 0.08, 0.05, 0.03, 0.02, 0.01, 0.008, 0.005, or no more than 0.001. In some instances, the p-value is about 0.5, 0.4, 0.3, 0.2, 0.15, 0.12, 0.1, 0.9, 0.8, 0.5, 0.4, 0.3, 0.2, 0.1, 0.08, 0.05, 0.03, 0.02, 0.01, 0.008, 0.005, or about 0.001. In some instances, the p-value is no more than 0.5, 0.4, 0.3, 0.2, 0.15, 0.12, 0.1, 0.9, 0.8, 0.5, 0.4, 0.3, 0.2, 0.1, 0.08, 0.05, 0.03, 0.02, 0.01, 0.008, 0.005, or no more than 0.001 and the expression level is increased at least 1.1, 1.2, 1.5, 1.7, 2, 3, 4, 5, 6, or at least 10 fold increase relative to a control. In some embodiments, the p value is less than 0.1 (i.e., p<0.1). In some other embodiments, the p value is less than 0.01 (i.e., p<0.01). In some embodiments, the fold change in gene expression between one or more detected genes is at least 1.1 fold, 1.2 fold, 1.3 fold, 1.4 fold, 1.5 fold, 2 fold, 4 fold, 6 fold, 10 fold, 15 fold, or 20 fold, or any measurement in between. In some other embodiments, the fold change in gene expression between one or more detected genes is at least 1.5 fold. In some embodiments, the fold change in gene expression between one or more detected genes is measured as an upregulation relative to a control. In some embodiments, the fold change in gene expression between one or more detected genes is measured as a down regulation relative to a control.

In some instances, a mutational change in one or more genes of interest from a biological sample comprises at least one mutation as compared to a control sample. In some instances, the mutational change is in TERT, CDKN2A, TP53, PTCH1, or a combination thereof. In some instances, TERT, CDKN2A, TP53, PTCH1, or a combination thereof from the biological sample comprises at least or about 1, 2, 3, 4, 5, 6, 7, 8, 9, 10, or more than 10 mutations. In some instances, TERT, CDKN2A, TP53, PTCH1, or a combination thereof from the biological sample comprises at least or about at least 1.5×, 2×, 3×, 4×, 5×, 6×, 7×, 8×, 9×, 10×, 11×, or 12× more mutations compared to a normal biological sample. In some instances, at least one of expression level and mutational change of a gene of interest provide information regarding a skin cancer. In some instances, the skin cancer is melanoma, basal cell carcinoma (BCC), or squamous cell carcinoma (SCC). In some instances, the skin cancer is BCC. In some instances, the skin cancer is SCC. For example, the at least one of expression level and mutational change of a gene of interest provide information regarding a stage of skin cancer. In some instances, the at least one of expression level and mutational change of a gene of interest is associated with a stage of skin cancer. In some instances, one or more mutations in a gene of interest indicate a risk factor for skin cancer or the stage of skin cancer. In some instances, the gene of interest is one or more of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, the gene of interest comprising a mutational change is at least one of TERT, CDKN2A, TP53, and PTCH1. In some instances, the gene of interest is one or more of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9-590, FOXI3-AP329YP, EDN1-961, MUC5B-588, WIP1-447, HLA-H-683, CDV3-555, FMNL3-999, STRA6-621, CASC15-411, FSTL1-496, ATG4B-774, KRT7-840, FDCSP-324, EAF1-532, or SLC5A10-293. In some instances, the gene of interest is one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, the gene of interest is one or more of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. In some instances, the gene of interest is one or more of MT-ND3, JPH3, TIMP2, MT-ND4, SOX11, and MED16. In some instances, the gene of interest is one or more of KRT33A, KRTAP8.1, KRTAP9-5, KRTAP2-2, KRTAP11-1, RTAP9-4, KRT33B, KRTAP1-5, KRTAP3-3, KRTAP2-1, KRTAP3-1, and KRTAP1-1. In some instances, the gene of interest is one or more of DSG4, GPRC5D, KRTAP4-11, KRTAP7-1, KRTAP8-1, KRTAP9-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, the gene of interest is one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, the gene of interest is one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, the gene of interest is one or more of KRT85, TIMP1, FP236383.5, LCE6A, LCE2C, LCE1F, FP236383.4, IL37, LCE5A, AL139247.1, WARS1, and GBP1. In some instances, the gene of interest is one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6.

Methods and compositions provided herein comprising detecting at least one of expression level and mutational change result in improved sensitivity and specificity for diagnosis or prognosis of disease. In some instances, detecting at least one of expression level and mutational change result in improved sensitivity and specificity for diagnosis or prognosis of skin cancer. In some instances, sensitivity is improved by at least or about 55%, 60%, 65%, 70%, 75%, 80%, 85%, 90%, 95% or more than 95% as compared to other diagnosis or prognosis methods. In some instances, specificity is improved by at least or about 55%, 60%, 65%, 70%, 75%, 80%, 85%, 90%, 95% or more than 95% as compared to other diagnosis or prognosis methods. The other diagnosis or prognosis methods include, but are not limited to, morphology histopathology, pattern histopathology, and RNA only based gene expression assays.

### Computer Implemented Methods and Systems for Carcinoma Assay

Described herein, in some embodiments, are non-transitory computer-readable medium with instructions stored thereon, that when executed by a processor, perform the steps comprising: (a) generating gene expression data of one or more of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 from a set of biological samples by a sequencing method; (b) obtaining, by a processor, the gene expression data; (c) generating a plurality of pair-wise interactions between at least two genes of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 from a set of biological samples by a sequencing method; (b) obtaining, by a processor, the gene expression data; (c) generating a plurality of pair-wise interactions between at least two genes of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. In some instances, the count value is measured in counts per million reads. In some instances, the machine learning method utilizes an algorithm selected from: random forest (rf) model, boosting model, logit model, or lasso model. In some instances, the set of samples comprises one or more of basal cell carcinoma, squamous cell carcinoma, actinic keratosis (AK), seborrheic keratosis (SK), normal samples, or a combination thereof. In some instances, the set of samples comprises one or more of basal cell carcinoma, squamous cell carcinoma, actinic keratosis (AK), seborrheic keratosis (SK), and normal samples. In some instances, the set of samples comprises one or more of squamous cell carcinoma and actinic keratosis (AK). In some instances, the set of samples comprises one or more of squamous cell carcinoma (ivSCC and isSCC) and actinic keratosis (AK). In some instances, the set of samples comprises ivSCC and isSCC samples. In some instances, the set of samples comprises basal cell carcinoma, squamous cell carcinoma, and actinic keratosis (AK). In some instances, the one or more cancer samples are differentiated from the one or more non-cancer samples when a log 2 threshold value is at least 1, 2, 3, 4, 5, 6, 7, 8, 9, 10, 12, 14, 15, 17, or 20. In some instances, the one or more cancer samples are differentiated from the one or more non-cancer samples when a log 2 threshold value is no more than −1, −2, −3, −4, −5, −6, −7, −8, −9, −10, −12, −14, −15, −17, or −20.

Described herein, in some embodiments, are computer-implemented methods for differentiating one or more cancer samples from one or more non-cancer samples, comprising: (a) hybridizing one or more probes that recognizes one or more of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. In some instances, the pair-wise interactions comprises IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2Am, MT-CO2, RNA5SP202, MIR4421, HAL, RNA5SP263, TIMP1, and RNA5SP481. Described herein, in some embodiments, are computer-implemented methods for differentiating one or more cancer samples from one or more non-cancer samples, comprising: (a) hybridizing one or more probes that recognizes one or more of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-12 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, the pair-wise interactions comprise two or more of MT-ND3, JPH3, TIMP2, MT-ND4, SOX11, and MED16. Described herein, in some embodiments, are non-transitory computer-readable medium with instructions stored thereon, that when executed by a processor, perform the steps comprising: (a) generating gene expression data of one or more of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 from a set of biological samples by a sequencing method; (b) obtaining, by a processor, the gene expression data; (c) generating a plurality of pair-wise interactions between at least two genes of MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of MT-ND3, JPH3, TIMP2, MT-ND4, SOX11, and MED16 from a set of biological samples by a sequencing method; (b) obtaining, by a processor, the gene expression data; (c) generating a plurality of pair-wise interactions between at least two genes of MT-ND3, JPH3, TIMP2, MT-ND4, SOX11, and MED16; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 from a set of biological samples by a sequencing method; (b) obtaining, by a processor, the gene expression data; (c) generating a plurality of pair-wise interactions between at least two genes of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77 from a set of biological samples by a sequencing method; (b) obtaining, by a processor, the gene expression data; (c) generating a plurality of pair-wise interactions between at least two genes of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1 from a set of biological samples by a sequencing method; (b) obtaining, by a processor, the gene expression data; (c) generating a plurality of pair-wise interactions between at least two genes of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of TAGLN, FDCSP, CASC15, CYP24A1, FOXI3, LINC02167, FSTL1, JPH3, CCT8, FMNL3, MED16, MT-ND3, STRA6, KIF1B, MIT-ND4, SOX11, and EDN1 from a set of biological samples by a sequencing method; (b) obtaining, by a processor, the gene expression data; (c) generating a plurality of pair-wise interactions between at least two genes of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of KRT85, TIMP1, FP236383.5, LCE6A, LCE2C, LCE1F, FP236383.4, IL37, LCE5A, AL139247.1, WARS1, and GBP1 from a set of biological samples by a sequencing method; (b) obtaining, by a processor, the gene expression data; (c) generating a plurality of pair-wise interactions between at least two genes of KRT85, TIMP1, FP236383.5, LCE6A, LCE2C, LCE1F, FP236383.4, IL37, LCE5A, AL139247.1, WARS1, and GBP1; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value. Described herein, in some embodiments, are non-transitory computer-readable medium with instructions stored thereon, that when executed by a processor, perform the steps comprising: (a) generating gene expression data of one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 from a set of biological samples by a sequencing method; (b) obtaining, by a processor, the gene expression data; (c) generating a plurality of pair-wise interactions between at least two genes of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value.

Described herein, in some embodiments, are non-transitory computer-readable medium with instructions stored thereon, that when executed by a processor, perform the steps comprising: (a) generating gene expression data of one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 from a set of biological samples by a sequencing method; (b) obtaining, by a processor, the gene expression data; (c) generating a plurality of pair-wise interactions between at least two genes of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7; (d) analyzing the plurality of pair-wise interactions using a machine learning method to determine a count value for each of the plurality of pair-wise interactions; and (e) differentiating the one or more cancer samples from the one or more non-cancer samples when a count value is greater than above a threshold value.

Described herein, in some embodiments, are computer-implemented methods for differentiating one or more cancer samples from one or more non-cancer samples, comprising: (a) hybridizing one or more probes that recognizes one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KR T85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, KRT7, and SPPG1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. In some instances, the pair-wise interactions comprises TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A1, METTL9, EAF1, CDV3, TIMP2, ZIC2, AKR1C1, HLA-H, MT-CYB, SLC5A10, and KRT7. Described herein, in some embodiments, are computer-implemented methods for differentiating one or more cancer samples from one or more non-cancer samples, comprising: (a) hybridizing one or more probes that recognizes one or more of MT-ND3, JPH3, TIMP2, MT-ND4, SOX11, MED16, KRT33A, KRTAP8-1, KRTAP19-5, KRTAP2-2, KRTAP11-4, KRTAP9-4, KRT33B, KRTAP1-5, KRTAP3-3, KRTAP2-1, KRTAP3-1, and KRTAP1-1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of MT-ND3, JPH3, TIMP2, MT-ND4, SOX11, MED16, KRT33A, KRTAP8-1, KRTAP19-5, KRTAP2-2, KRTAP11-4, KRTAP9-4, KRT33B, KRTAP1-5, KRTAP3-3, KRTAP2-1, KRTAP3-1, and KRTAP1-1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. In some instances, the pair-wise interactions comprises MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1. Described herein, in some embodiments, are computer-implemented methods for differentiating one or more cancer samples from one or more non-cancer samples, comprising: (a) hybridizing one or more probes that recognizes one or more of MT-ND3, JPH3, TIMP2, MT-ND4, SOX11, and MED16 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of MT-ND3, JPH3, TIMP2, MT-ND4, SOX11, and MED16 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises MT-ND3, JPH3, TIMP2, MT-ND4, SOX11, and MED16. In some instances, the pair-wise interactions comprises MT-ND3, JPH3, TIMP2, MT-ND4, SOX11, and MED16. Described herein, in some embodiments, are computer-implemented methods for differentiating one or more cancer samples from one or more non-cancer samples, comprising: (a) hybridizing one or more probes that recognizes one or more of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, the pair-wise interactions comprises KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. Described herein, in some embodiments, are computer-implemented methods for differentiating one or more cancer samples from one or more non-cancer samples, comprising: (a) hybridizing one or more probes that recognizes one or more of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises DSG4, GPRC5D, KRTAP4-11, KRTAP7-1, KRTAP8-1, KRTAP9-5, KRTAP9-1, KRTAP9-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. In some instances, the pair-wise interactions comprises DSG4, GPRC5D, KRTAP4-11, KRTAP7-1, KRTAP8-1, KRTAP9-5, KRTAP9-1, KRTAP9-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77. Described herein, in some embodiments, are computer-implemented methods for differentiating one or more cancer samples from one or more non-cancer samples, comprising: (a) hybridizing one or more probes that recognizes one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. In some instances, the pair-wise interactions comprises TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1. Described herein, in some embodiments, are computer-implemented methods for differentiating one or more cancer samples from one or more non-cancer samples, comprising: (a) hybridizing one or more probes that recognizes one or more of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. In some instances, the pair-wise interactions comprises TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1. Described herein, in some embodiments, are computer-implemented methods for differentiating one or more cancer samples from one or more non-cancer samples, comprising: (a) hybridizing one or more probes that recognizes one or more of KRT85, TIMP1, FP236383.5, LCE6A, LCE2C, LCE1F, FP236383.4, IL37, LCE5A, AL139247.1, WARS1, and GBP1 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of KRT85, TIMP1, FP236383.5, LCE6A, LCE2C, LCE1F, FP236383.4, IL37, LCE5A, AL139247.1, WARS1, and GBP1 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises KRT85, TIMP1, FP236383.5, LCE6A, LCE2C, LCE1F, FP236383.4, IL37, LCE5A, AL139247.1, WARS1, and GBP1 In some instances, the pair-wise interactions comprises KRT85, TIMP1, FP236383.5, LCE6A, LCE2C, LCE1F, FP236383.4, IL37, LCE5A, AL139247.1, WARS1, and GBP1 Described herein, in some embodiments, are computer-implemented methods for differentiating one or more cancer samples from one or more non-cancer samples, comprising: (a) hybridizing one or more probes that recognizes one or more of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 to generate gene expression data; (b) obtaining, by a processor, the gene expression data; and (c) analyzing, by the processor, the gene expression data to differentiate the one or more cancer samples from the one or more non-cancer samples, wherein the analysis comprises: (i) generating a plurality of pair-wise interactions between at least two genes of KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 in a set of samples; (ii) analyzing the plurality of pair-wise interactions using a machine learning method to determine an area under a curve (AUC) value for each of the plurality of pair-wise interactions; and (iii) differentiating the one or more cancer samples from the one or more non-cancer samples when an AUC value is greater than above about 0.8. In some instances, the pair-wise interactions comprises KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6. In some instances, the pair-wise interactions comprises KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KR T28, RNF5, IGF2BP2, TIMP1, EDN1, and KRT16P6.

In some instances, the machine learning method utilizes an algorithm selected from: random forest (rf) model, boosting model, logit model, or lasso model. In some instances, the set of samples comprises basal cell carcinoma, squamous cell carcinoma, actinic keratosis (AK), seborrheic keratosis (SK), normal samples, or a combination thereof. In some instances, the set of samples comprises basal cell carcinoma, squamous cell carcinoma, actinic keratosis (AK), seborrheic keratosis (SK), and normal samples. In some instances, the set of samples comprises one or more of squamous cell carcinoma and actinic keratosis (AK). In some instances, the set of samples comprises one or more of squamous cell carcinoma (ivSCC and isSCC) and actinic keratosis (AK). In some instances, the set of samples comprises ivSCC and isSCC samples. In some instances, the set of samples comprises basal cell carcinoma, squamous cell carcinoma, and actinic keratosis (AK). In some instances, the set of samples comprises basal cell carcinoma and squamous cell carcinoma. In some instances, the one or more cancer samples are differentiated from the one or more non-cancer samples when an AUC value is greater than above about 0.6, 0.65, 0.7, 0.75, 0.8, 0.85, 0.9, or 0.95.

### Biological Samples and Methods of Use

Biological samples are obtained using a variety of methods. In some instances, obtaining a biological sample such as a skin sample comprises, but is not limited to, scraping of the skin, biopsy, suction, blowing and other techniques. In some instances, obtaining the biological sample is non-invasive. For example, the biological sample is obtained from a skin using a skin sample collector. In some cases, the biological sample is obtained by applying an adhesive patch to a skin sample in a manner sufficient to adhere a sample of the skin to the adhesive patch, and removing the adhesive patch from the skin in a manner sufficient to retain the adhered skin sample to the adhesive patch. In some instances, the patch comprises a rubber adhesive on a polyurethane film. In some instances, about 1, 2, 3, 4, 8, 12, 16, 20, 24, or 32 patches are applied to and removed from the skin. In some instances, about one to about ten or twelve adhesive patches or one to ten or twelve applications of the patch are applied to and removed from the skin. In some instances, samples are collected serially (i.e., from approximately the same area of skin or same lesion). In some instances, at least 1, 2, 3, 4, 8, 12, 16, 20, 24, 32 or more than 32 patches are applied to approximately the same area of skin or same lesion. In some instances, samples are collected from different areas of the skin on the body. In some instances, at least 1, 2, 3, 4, 8, 12, 16, 20, 24, 32 or more than 32 patches are applied to different areas of the body. In some instances, samples are collected serially (i.e., from approximately the same area of skin or same lesion). In some instances, about 1, 2, 3, 4, 8, 12, 16, 20, 24, 32 about 40 patches are applied to approximately the same area of skin or same lesion. In some instances, samples are collected from different areas of the skin on the body. In some instances, about 1, 2, 3, 4, 8, 12, 16, 20, 24, 32 about 40 patches are applied to different areas of the body. In some instances, 1-40, 1-30, 1-20, 1-15, 1-12, 1-8, 1-4, 2-8, 2-16, 4-8, 4-12, 8-12, 8-16, 8-24, or 12-24 patches are applied to approximately the same area of skin or same lesion. In some instances, samples are collected from different areas of the skin on the body. In some instances, 1-40, 1-30, 1-20, 1-15, 1-12, 1-8, 1-4, 2-8, 2-16, 4-8, 4-12, 8-12, 8-16, 8-24, or 12-24 patches are applied to different areas of the body. In some instances about 4 patches are applied to the skin, either at the same location or at different locations on the body. In some instances about 4 patches are serially applied to the skin at the same location on the body. In some instances, about 4 patches are consecutively applied to the skin at different locations on the body. The used patch, in some instances, is stored so that the matrix containing, skin facing surface of the used patch is in contact with the placement area sheet. In some instances, the placement area sheet is a panel of the skin sample collector. In some instances, the skin sample collector further comprises a clear panel. In some instances, the skin sample collector is labeled with a unique barcode that is assigned to a subject. In some instances, the skin sample collector comprises an area for labeling subject information.

In an illustrative embodiment, the adhesive skin sample collection kit comprises one or more of: one or more adhesive patches, a peelable release panel, bag for storing the sampling, preservative, and a unique label. In some instances, the bag comprises a foil bag. In some instances, the bag comprises a plastic bag. In some instances, the collection kit comprises a foil bag and a plastic bag. In some instances, the preservative comprises a desiccant. In some instances, a skin collection kit does not comprise a desiccant.

Skin sample collection kits provided herein may comprise unique identifiers. In some embodiments, identifiers uniquely identify one or more of a patient, insurance carrier, doctor, sampling location (e.g., hospital, clinic, home, or other location), sample kit, sampling location from the body (e.g., chest, arms, face, head, back, etc.), destination laboratory for analysis, sample time, requested analysis method, and other property associated with the sample or testing method. In some instances, identifiers are printed on the collection kit or label thereon. In some instances, printed identifiers comprise codes (e.g., letters, numbers, symbols, etc.), scannable barcodes (e.g., 1D or 2D QR codes). In some instances, identifiers comprise magnetic or RFID identifiers. In some instances, identifiers are read with a scanner.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, foil bag, gloves, desiccant, unique label(s), and a cleansing reagent. The cleansing reagent includes, but is not limited to, an antiseptic such as isopropyl alcohol. In some instances, the components of the skin sample collection kit are provided in a cardboard box. In some instances, a skin collection kit does not comprise a desiccant.

Provided herein are systems and devices for collecting skin samples (skin sample collector). In some instances, a skin sample collector (or system) comprises one or more adhesive patches (tapes, stickers, strips, or other collector). In some instances, an adhesive patch comprises one or more of: a backing layer, an adhesive matrix, and a non-invasive handling area. In some instances, a skin sample collector further comprises one or more of a release panel, individual liners, a placement area, and individual panels. In some instances, devices are configured for optimum peel adhesion, elasticity of the backing film, extractables, dimensions, materials, functional results, or a combination thereof. In some instances, the backing layer comprises a flexibility and/or elasticity to conform to a morphology of a portion of skin comprising a lesion, and wherein the backing layer comprises a thickness such the at least one adhesive patch resists wrinkling. In some instances, the backing layer comprises a combination of dimensions, flexibility, and/or elasticity such that the at least one adhesive patch resists wrinkling. The wrinkling may be static wrinkling, such as wrinkling when a patch is on the skin. The wrinkling may be dynamic wrinkling, such as wrinkling when the at least one adhesive patch is released from the skin. In some instances, the at least one patch comprises a thickness such that it does not self-adhere when supported by a portion of the non-adhesive handling layer with a draft and in multiple orientations. In some instances, an amount of extractables and leachables released from the at least one adhesive patch is minimized to improve nucleic acid analysis. In some instances, the at least one adhesive patch comprises a longest dimension of about a wrinkling wavelength of the at least one adhesive patch. In some instances, the adhesive matrix comprises a pressure sensitive adhesive, wherein the pressure sensitive adhesive exhibits a glass transition temperatures lower than 5° C.

The adhesive patch of the adhesive skin sample collector typically comprises a backing layer. In some instances, the backing area comprises a first collection area comprising an adhesive matrix and a second area extending from the periphery of the first collection area. The adhesive matrix is located on a skin facing surface of the first collection area. The second area functions as a tab (or non-adhesive handling area), suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use. In some instances, the backing comprises a soft, clear, and pliable synthetic polymer.

The backing layer may comprise any material or mixture of materials which controls rigidity or flexibility. Without being bound by theory, a backing layer enables proper conformation of the patch over the lesion of any size or shape, which leads to higher removal of cellular materials during peeling off/collection. In some instances, the thickness or rigidness of the backing layer is configured to prevent deformation due to static wrinkles or slip-stick patterns during peel. In some embodiments, the backing layer comprises a polyurethane carrier film. Patches described herein may comprise any number of materials which provide for the desired sampling properties (e.g., thickness, performance, patient comfort, or other property).

The backing layer may comprise materials or mixtures of materials selected to mitigate wrinkling of the backing layer. Wrinkling of the backing layer may be characterized by a wrinkling pattern. In some cases, the wrinkling pattern may be a regular pattern. The wrinkling pattern may be irregular. A pattern of the wrinkling may be characterized by a wrinkling wavelength (e.g., an average wavelength). The wrinkling wavelength may be a distance (e.g., an average distance) between subsequent peaks or subsequent troughs in the wrinkles. In some instances, wrinkling comprises the average distance between the peak points of the periodic (and standing) wavy structures formed on the skin when a stiffer tape is applied on typically softer skin. An average wavelength may be determined from an average distance between peaks for the length of the tape. Wrinkling may be static or dynamic. Static wrinkling may occur when a backing layer comprising an adhesive is adhered to a surface (e.g., a skin). Dynamic wrinkling may occur during peeling of the backing layer. In some instances, the wrinkling wavelength approaches the length of a patch, such as 50%, 70%, 90%, 95%, 97%, 98%, 99%, 99.5%, or 99.9% of the length of a patch. In some instances, wrinkling is increased with the use of higher flexibility backing layers. In some instances, backing layers of at least 1, 2, 3, 4, 5, 6, or more than 6 mils result in a reduction in wrinkling.

In some cases, dynamic wrinkling may be caused by sticking and slipping of the backing layer during peeling. The process of peeling a backing layer comprising an adhesive may include dynamic sticking and slipping. For example, even when a user endeavors to peel a backing layer as smoothly as possible, the peeling may stop and start causing the effect of sticking and slipping. For example, during a stick, elastic potential energy may be stored in the adhesive and the bend of the tape. In some cases, both the tape and the adhesive may act like springs that store energy as they are stretched. During a slip, potential energy may be converted to kinetic energy. The sticking and slipping may occur even on microscopic length scales (e.g. length scales on the order of few microns or greater). Sticking and slipping may result in defects (e.g., wrinkles) during a peeling step. While not wishing to be bound by theory, the frequency of the stick-slip patterns in some instances decreases with the square root of the patch thickness. For example, the modulus of elasticity of the backing sheet may at least partially govern the wrinkling wavelength by the square root of the cubic root, which provides an exponent of ⅙, (i.e. λ˜Et1/6).

Parameters which effect the sticking and slipping may include elasticity of the skin, elasticity of the backing layer, strength of the adhesive, and geometric parameters such as the length and width of the tape. One or more of these parameters may affect a wavelength and frequency of wrinkling patterns in the backing layer. The skin elasticity may relate to the potential energy stored in a stick. For example, skin with a high elasticity may store greater potential energy during a stick and slip to a greater distance. The elasticity of the backing layer may relate to the potential energy stored in a stick. For example, a backing layer with a high elasticity may store greater potential energy during a stick and slip to a greater distance. The adhesive may relate to the potential energy stored in a stick. For example, a stronger adhesive may store greater potential energy during a stick and slip to a greater distance. In some cases, a separation front, the line dividing the attached portion to the separated portion, may not be a straight line during slips. For example, a slip may propagate along a width of the backing layer if the peel is along a length of the backing layer. Accordingly, a wider tape may change the wrinkling properties of the tape by changing the slip dynamics and/or by increasing the potential energy to peel per unit distance peeled along the peeling axis. In some examples, the wrinkling wavelength may be on the order of several centimeters. A wrinkling wavelength which is longer than the backing layer may mitigate dynamic wrinkles.

Static wrinkling may occur when an adhesive patch is attached to the skin. In some cases, static wrinkling may be caused by a mismatch between the extent of contraction of the soft foundation (e.g., skin) and the harder surface (e.g., the backing layer of the tape) due to the in-plane forces exerted by the adhesive. Parameters which effect the static wrinkling may include elasticity of the skin, elasticity of the backing layer, strength of the adhesive, and geometric parameters such as the length and width of the tape. One or more of these parameters may affect a wavelength and frequency of wrinkling patterns in the backing layer. The extent of contraction of the soft foundation may be related to the elasticity of the soft foundation. The extent of contraction of the harder surface may be related to the elasticity of the backing layer. A mismatch between the extents of contraction may create a deformation in the peel (e.g., a wrinkle). The deformation may be characterized by an amplitude. A mismatch between the extents of contraction may cause static wrinkles. The frequency of static wrinkles may be strongly correlated with the thickness of the backing layer. In some examples, the wrinkling wavelength may be on the order of several centimeters. A wrinkling wavelength which is longer than the backing layer may mitigate static wrinkles. In some cases, a backing layer with a thickness greater than 3 mil or above may provide a wrinkling wavelength of several centimeters.

In some embodiments, the wrinkling wavelength is configured to mitigate static and/or dynamic wrinkling. In some examples, the wrinkling wavelength may be on the order of several centimeters. A wrinkling wavelength that is longer than a length of the backing layer may mitigate wrinkling. A wrinkling wavelength that is longer than a length of a patch applied to the skin may mitigate wrinkling. The wrinkling wavelength may comprise a length which is equal to or greater than, for example and without limitation, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm.

In some instances, patches described herein comprise a backing layer. In some embodiments, the backing layer comprises one or more of TPU (thermoplastic polyurethane), LPDE (low density polyethylene), PET (polyethylene), PP (polypropylene), Teflon, Polyimide, PEN (Polyethylene naphthalate), PVB (polyvinyl butyral), PVOH (poly(vinyl alcohol)), PVP (Poly(vinylpolypyrrolidone)) cellulose butyrate, cellulose acetate, or a mixture thereof. In some embodiments, the backing layer comprises TPU (thermoplastic polyurethane) and LPDE (low density polyethylene). In some instances, the soft, clear, and pliable synthetic polymer comprises an elastomer of olefin. In some instances, the elastomer of olefin comprises copolymers or compounds of polymers comprising one or more of ethylene, propylene, isobutylene, vinyl acetate, vinyl alcohol, ethylene oxide, and propylene oxide. In some instances, the soft clear, and pliable synthetic polymer comprises a thermoplastic elastomer. In some instances, the thermoplastic elastomer comprises a polyester based elastomer. In some instances, the thermoplastic elastomer comprises a copolymer or compound of an ether or amide.

In some instances, flexibility is controlled by properties of the backing layer, the adhesive matrix, or both. In some instances, patches are configured to adhere to atypical/3-dimensional morphologies. In some instances, patches comprise a conformability/flexibility to contact the morphological structure of the lesion while minimizing or avoiding wrinkling of the patch upon peel/release. In some instances, flexibility and the thickness of the backing layer provides for the proper conformation of the patch over the lesion of any size or shape, which leads to higher removal of skin cells during peeling off/collection. In some instances, flexibility is measured using ASTM D882 or ASTM D1938 methods with an XLW (EC) Auto Tensile Tester (Labthink Instrument Inc). In some instances, the thickness of the backing layer is no more than 7, 6, 5, 4, 3, 2.5, 2.0, 1.5, 1.25, 1, 0.8, 0.7, 0.6, 0.5, 0.3, 0.2, or no more than 0.1 mils. In some instances, the thickness of the backing layer is about 7, 6, 5, 4, 3, 2.5, 2.0, 1.5, 1.25, 1, 0.8, 0.7, 0.6, 0.5, 0.3, 0.2, or about 0.1 mils. In some instances, the thickness of the backing layer is 0.1-5, 0.1-4, 0.1-3, 0.1-2, 0.1-1, 0.5-4, 0.5-3, 1-5, 2-7, 3-5, 3-10, or 1-2 mils. In some instances, a backing layer comprising one or more of LDP or TPU has a thickness of at least 1, 2, 2.5, 3, 3.5, 4, 4.5, 5, 5.5, or more than 6 mils.

In some instances, elasticity is controlled by properties of the backing layer, the adhesive matrix, or both. In some instances, patches are configured to adhere to atypical/3-dimensional morphologies. In some instances, patches comprise an elasticity to contact the morphological structure of the lesion while minimizing or avoiding wrinkling of the patch upon peel/release. The elasticity may be characterized by an elastic modulus. In some instances, the backing layer has an elastic modulus from about 200 to about 2,000 Psi as measured by ASTM D-882. In some instances, the backing layer has an elastic modulus of about 250, 500, 750, 1000, 1250, 1500, 1750, 2000, 2250, 2500, 3000, 3250, 3500 or about 4000 Psi. In some instances, the backing layer has an elastic modulus of from about 1000 to about 2000 Psi, about 500 to about 3000 Psi, about 250 to about 2000 Psi, about 400 to about 2000 Psi, about 500 to about 1500 Psi, about 750 to about 2000 Psi, about 1000 to about 3000 Psi, about 1000 to about 4000 Psi, about 2000 to about 4000 Psi, or about 500 to about 2500 Psi. In some instances, the backing layer has a tensile strength of from about 7 to about 60 MPa, about 5 to about 60 MPa, about 10 to about 60 MPa, about 20 to about 80 MPa, about 30 to about 60 MPa, about 5 to about 30 MPa, about 5 to about 20 MPa, or about 7 to about 15 MPa. In some instances, the backing layer has an elongation of 100-1000%, 100-750%, 100-500%, 150-500%, 200-1000%, 400-600%, 400-800%, 500-1000%, 750-1000%, or 750-1500%. Tensile strength and/or elongation in some instances is measured using CD (cross-direction) or MD (machine direction) test values.

Adhesive patches described herein may comprise an adhesive matrix. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent. In some instances, the adhesive matrix comprises one or more of acrylics, silicones and hydrocarbon rubbers (like butyl rubber, styrene-butadiene rubber, ethyl-vinyl acetate polymers, styrene-isoprene-butadiene rubbers), or combination thereof. In some instances, tack of the adhesive matrix is measured by ASTM D1876 using XLW (EC) Auto Tensile Tester (Labthink Instrument Inc). In some instances, the adhesive matrix comprises a hydrophobicity of no more than 2000, 1500, 1000, 900, 800, 700, 600, 500, 400, 300, 200, or no more than 150 g/m²/24 hours. In some instances, hydrophobicity is measured as an upright MVTR (moisture vapor transmission rate) or inverted MVTR. In some instances, hydrophobicity is measured using ASTM E96-80. In some instances, the patch (including adhesive matrix) comprises a hydrophobicity of no more than 2000, 1500, 1000, 900, 800, 700, 600, 500, 400, 300, 200, or no more than 150 g/m²/24 hours. In some instances, the adhesive matrix comprises a peel adhesion, or force exerted when removing a patch comprising the adhesive matrix. In some instances, peel adhesion is optimal when the desired amount of cellular material is removed from the skin, but without causing skin damage or discomfort to the patient. In some instances, the peel adhesion is measured using ASTM D3330. In some instances, peel adhesion is measured using PSTC-1. In some instances, the peel adhesion is 1-40, 1-30, 1-20, 5-30, 5-25, 5-20, 5-15, 3-15, 3-12, 10-20, 5-30, 15-30, or 3-10 Newtons/inch. In some instances, the peel adhesion is at least 1, 2, 3, 4, 5, 6, 7, 8, 9, 10, 12, 15, 20, 25, 30, or at least 35 Newtons/inch. In some instances, the peel adhesion is no more than 1, 2, 3, 4, 5, 6, 7, 8, 9, 10, 12, 15, 20, 25, 30, or no more than 35 Newtons/inch. In some instances, the peel adhesion is about 1, 2, 3, 4, 5, 6, 7, 8, 9, 10, 12, 15, 20, 25, 30, or about 35 Newtons/inch. In some instances, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 1800 peel adhesion at a pull rates from about 1.0 inch/min to about 12.0 inch/min. In some instances, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 1800 peel adhesion at a pull rates from about 4.0 inch/min to about 16.0 inch/min. In some instances, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 1800 peel adhesion at a pull rates from about 0.5 inch/min to about 8 inch/min. In some instances, the adhesive matrix comprises a pressure sensitive adhesive. In some instances, the pressure sensitive adhesive exhibits a glass transition temperature lower than 20° C., 15° C., 10° C., 7° C. 6° C., 5° C., 4° C., 3° C., or lower than 2° C. In some instances, the pressure sensitive adhesive exhibits a glass transition temperature of 1-20° C., 1-15° C., 1-10° C., 1-7° C. 3-8° C., 4° C.-6° C. or 4° C.-10° C. In some instances, the pressure sensitive adhesive exhibits a glass transition temperature of about 20° C., 15° C., 10° C., 7° C., 6° C., 5° C., 4° C., 3° C., or about 2° C. In some instances, pressure-sensitive tack of an adhesive is measured. In some instances, pressure-sensitive tack of an adhesive is measured using ASTM D2979. In some instances, pressure-sensitive tack of the adhesive is 100-200, 100-500, 100-750, 100-1000, 150-500, 150-300, 200-500, 200-750, 300-400, 300-600, 450-750, or 500-1000 grams per square inch. In some instances, pressure-sensitive tack of the adhesive is about 50, 75, 100, 125, 150, 175, 200, 250, 300, 350, 400, 500, 600, 700, 800, or about 1000 grams per square inch.

Adhesives may be configured to reduce wrinkling during skin patch sampling. In some instances, provided herein are patches comprising an adhesive. In some instances, provided herein are patches comprising a hybrid adhesive (comprising two or more components). In some instances, adhesives comprise-pressure sensitive adhesives. In some instances, the adhesive comprises a component selected from two or more of silicone, acrylate polymer, or rubber (natural or synthetic). In some instances, acrylic polymer comprises “pure” acrylic or modified acrylic adhesives. In some instances, synthetic rubber comprises hot-melt rubber, solvent rubber, or butyl rubber. In some instances, the adhesive comprises one or more components. In some instances, the adhesive comprises a first component, wherein the first component comprises a synthetic rubber adhesive. In some instances, the adhesive comprises a second component, wherein the second component comprises an acrylate polymer. In some instances, the adhesive is applied to patch comprising a polyester backing layer. Without being bound by theory, using a hybrid adhesive which rapidly reaches its maximal adhesion in some instances reduces or eliminates the skin sampling variations caused by operators, while the stiffer polyester backing creates a more uniformed skin sample collection across the patch stripping (i.e., reduces wrinkling). In some instances, adhesive components are homogenous. In some instances, adhesives comprise a first layer comprising a first component and a second layer comprising a second component. In some instances, adhesives comprise a first layer comprising a first component and a second layer comprising a second component, wherein the first layer comprises a rubber adhesive and the second layer comprises an acrylic adhesive.

Adhesives may comprise compositions as described in U.S. Pat. No. 5,625,005, incorporated herein by reference in its entirety. In some instances, adhesives comprise graft copolymer acrylates. In some instances, adhesives are generated by reacting at least one alkyl acrylate ester containing from about 4 to about 8 carbon atoms in the alkyl group in the presence of a macromer selected from the group consisting of ethylene-butylene and ethylene-propylene macromers and mixtures thereof, each of said macromers having a molecular weight of from about 2,000 to about 30,000. In some instances, adhesives comprise on a percent-by-weight basis, from about 35 to about 100 percent by weight of the total acrylate backbone of one or more alkyl acrylate esters (or vinyl esters) containing about 4 to about 8 carbon atoms in the alkyl group. In some instances, alkyl acrylate esters include n-butyl acrylate, 2-ethyl hexyl acrylate, and isooctyl acrylate. In some instances, vinyl esters include vinyl acetate, vinyl butyrate, vinyl propionate, vinyl isobutyrate, vinyl valerate, and vinyl versitate.

Adhesives may comprise compositions as described in U.S. Pat. No. 6,642,298, incorporated herein by reference in its entirety. In some instances, adhesives comprise an acrylic polymer copolymerized with a rubber macromer. In some instances, the polymer comprises at least one alkyl acrylate monomer containing from about 4 to about 18 carbon atoms in the alkyl group and at least one monomer whose homopolymer has a glass transition temperature greater than about 0° C., and wherein the macromer has a glass transition temperature of about −30° C. or less. In some instances, an adhesive comprises an acrylic polymer copolymerized with a rubber macromer (macromer), the polymer comprising at least one alkyl acrylate monomer containing from about 4 to about 18 carbon atoms in the alkyl group, and wherein the polymer is crosslinked using a titanium crosslinking agent. In some instances, the macromer comprises poly(ethylene-butylene), poly(ethylene-propylene) or poly(ethylene-butylene-propylene). In some instances, the macromer has a molecular weight of from about 2,000 to about 10,000. In some instances, the adhesive comprises methyl acrylate and hydroxyethyl acrylate or hydroxypropyl methacrylate In some instances, at least one alkyl acrylate monomer is 2-ethylhexyl acrylate, said at least one monomer is methyl acrylate and said at least one hydroxy functional monomer is hydroxyethyl acrylate.

Adhesives may comprise compositions as described in U.S. Pat. No. 7,396,871, incorporated herein by reference in its entirety. In some instances, the adhesive comprises a rubber modified acrylic and/or vinyl resin comprising the mini-emulsion polymerization product of at least one rubber compound substantially dissolved in at least one acrylic and/or vinyl monomer, wherein said resin comprises a rubber portion derived from said rubber compound and an acrylic and/or vinyl portion derived from said acrylic and/or vinyl monomer. In some instances, the at least one rubber compound is selected from one or more of the group consisting of natural rubber, butyl rubber, isoprene rubber, chloroprene rubber, neoprene rubber, polybutadiene rubber, nitrile-butadiene rubber, styrene-butadiene rubber, polypentanamer, and ethylene-propylene-diene terpolymer. In some instances, the acrylic monomer and/or vinyl monomer is selected from the group consisting of styrene, a-methyl styrene, vinyl naphthalene, vinyl toluene, chloromethyl styrene, methyl acrylate, acrylic acid, methacrylic acid, methyl methacrylate, ethyl acrylate, ethyl methacrylate, butyl acrylate, butyl methacrylate, isobutyl acrylate, isobutyl methacrylate, ethylhexyl acrylate, ethylhexyl methacrylate, lauryl methacrylate, lauryl acrylate, octyl acrylate, octyl methacrylate, glycidyl methacrylate, allyl methacrylate, vinyl methacrylate, acetoacetoxyethyl acrylate, acetoacetoxyethyl methacrylate, acetoacetoxypropyl acrylate, acetoacetoxypropyl methacrylate, hydroxybutenyl methacrylate, the allyl ester of maleic acid, the diallyl ester of maleic acid, poly(allyl glycidyl ether), alkyl crotonates, vinyl cetate, di-n-butyl maleate, di-octylmaleate, acrylonitrile, diacetone acrylamide, acrylamide, methacrylamide, hydroxyethyl methacrylate, hydroxyethyl acrylate, acrylonitrile, t-butylaminoethyl methacrylate, dimethylaminoethyl methacrylate, diethylaminoethyl methacrylate, N, N-dimethylaminopropyl methacrylamide, 2-t-butylaminoethyl methacrylate, N, N-dimethylaminoethyl acrylate, N-(2-methacryloyloxy-ethyl)ethylene urea, and methacrylamidoethylethylene urea.

Adhesives may comprise compositions as described in US 2008/0251201, incorporated herein by reference in its entirety. In some instances, adhesives comprise general compositions of poly(meth)acrylate; polyvinyl ether; diene rubber such as natural rubber, polyisoprene, and polybutadiene; polyisobutylene; polychloroprene; butyl rubber; butadiene-acrylonitrile polymer; thermoplastic elastomer; block copolymers such as styrene-isoprene and styrene-isoprene-styrene (SIS) block copolymers, ethylene-propylene-diene polymers, and styrene-butadiene polymers; poly-alpha-olefin; amorphous polyolefin; silicone; ethylene-containing copolymer such as ethylene vinyl acetate, ethylacrylate, and ethyl methacrylate; polyurethane; polyamide; epoxy; polyvinylpyrrolidone and vinylpyrrolidone copolymers; polyesters; and mixtures or blends of the above. Adhesives in some instances comprise additives including, but not limited to, tackifiers, plasticizers, fillers, antioxidants, stabilizers, pigments, diffusing materials, curatives, fibers, filaments, and solvents.

Adhesives are in some instances an acrylic based adhesive but other adhesives are contemplated as well and may be used. Such other adhesives include those based on silicones or based on polyolefins as disclosed in Handbook of Pressure Sensitive Adhesive Technology (third edition) D. Satas, Ed. Satas and Associates, Warwick R.I./USA, 1989 on pages 550-556 and 423-442 respectively.

Adhesives may comprise compositions as described in WO 2014/130507, incorporated herein by reference in its entirety. In some instances, patches described herein comprise one or more adhesive layers. In some instances, patches comprise a first adhesive layer and a second adhesive layer. In some instances, the first adhesive layer comprises an acrylic based adhesive, a rubber based adhesive, or a combination of two or more thereof. In some instances, the first layer comprises polyisoprene, polybutadiene, styrenebutadiene polymers, styrene-butadiene block copolymers, multi-armed repeating styrene-butadiene copolymers, styrene-isoprene-styrene polymers, styrene-butadienestyrene polymers, styrene-isoprene polymers, styreneisoprene block copolymers, and multi-armed repeating styrene-isoprene copolymers, or a combination of two or more thereof. In some instances, the second layer comprises an adhesive comprising a monomer chosen from methyl acrylate, ethyl acrylate, n-propyl acrylate, isopropyl acrylate, n-butyl acrylate, isobutyl acrylate, n-amyl acrylate, isoamyl acrylate, n-hexyl acrylate, isohexyl acrylate, cyclohexyl acrylate, isooctyl acrylate, 2-ethyl hexyl acrylate, decyl acrylate, lauryl acrylate, stearyl acrylate, isobornyl acrylate, methyl methacrylate, ethyl methacrylate, n-propyl methacrylate, isopropyl methacrylate, n-butyl methacrylate, isobutyl methacrylate, n-amyl methacrylate, isoamyl methacrylate, n-hexyl methacrylate, isohexyl methacrylate, cyclohexyl methacrylate, isooctyl methacrylate, 2-ethyl hexyl methacrylate, decyl methacrylate, lauryl methacrylate, stearyl methacrylate, and isobornyl methacrylate, or a combination of two or more thereof.

Adhesive patches may be transparent or opaque depending on the application. In some instances, the patch is opaque. In some instances, the patch is clear. In some instances, the patch has an opacity of about 1%, 2%, 5%, 8%, 10%, 15%, 20%, 25%, 30%, 40%, 50%, 60%, 70%, 80%, 90%, 95%, or about 98%. In some instances, the patch has an opacity of at least 1%, 2%, 5%, 8%, 10%, 15%, 20%, 25%, 30%, 40%, 50%, 60%, 70%, 80%, 90%, 95%, or at least 98%. In some instances, the patch has an opacity of no more than 1%, 2%, 5%, 8%, 10%, 15%, 20%, 25%, 30%, 40%, 50%, 60%, 70%, 80%, 90%, 95%, or no more than 98%. In some instances, the patch has an opacity after removing skin cells one or more times (peeling). In some instances, the patch has an opacity of about 1%, 2%, 5%, 8%, 10%, 15%, 20%, 25%, 30%, 40%, 50%, 60%, 70%, 80%, 90%, 95%, or about 98% after 1 peeling of skin cells. In some instances, the patch has an opacity of at least 1%, 2%, 5%, 8%, 10%, 15%, 20%, 25%, 30%, 40%, 50%, 60%, 70%, 80%, 90%, 95%, or at least 98% after 1 peeling of skin cells. In some instances, the patch has an opacity of no more than 1%, 2%, 5%, 8%, 10%, 15%, 20%, 25%, 30%, 40%, 50%, 60%, 70%, 80%, 90%, 95%, or no more than 98% after 1 peeling of skin cells. In some instances, an adhesive patch comprises a haze value of less than about 50%, 45%, 40%, 30%, 25%, 20%, 15%, 10%, or less than about 5% as measured by ASTM D1003.

Adhesive patches may comprise a matrix material. The matrix material in some instances is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant. In some instances, patches are applied multiple times to an area or region. In some instances, greater than 2 applications in the same area or region results in no more than 80, 70, 60, 50, 40, 35, 30, 25, 20, 17, 15, 12, 10, or no more than 5 g/m²/h) transepidermal water loss (TEWL). In some instances, greater than 4 applications in the same area or region results in no more than 80, 70, 60, 50, 40, 35, 30, 25, 20, 17, 15, 12, 10, or no more than 5 g/m²/h) transepidermal water loss (TEWL). In some instances, greater than 8 applications in the same area or region results in no more than 80, 70, 60, 50, 40, 35, 30, 25, 20, 17, 15, 12, 10, or no more than 5 g/m²/h) transepidermal water loss (TEWL). In some instances, greater than 6 applications in the same area or region results in no more than 80, 70, 60, 50, 40, 35, 30, 25, 20, 17, 15, 12, 10, or no more than 5 g/m²/h) transepidermal water loss (TEWL).

Adhesive patches may comprise a flexible material, enabling the patch to conform to the shape of the skin surface upon application (or backing layer). In some instances, patches comprise an adhesive matrix present in the first collection area. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical. In some embodiments the length of the patch (including both adhesive and non-adhesive handling areas) is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical. Without being bound by theory the length of a patch applied to the skin is comparable to the wrinkling wavelength to avoid the wavy structure on static patch before peel. In some instances, the longest linear dimension of the patch no more than 15, 12, 10, 8, 6, 5, 4, 3, 2, or no more than 1 cm. In some instances, the longest linear dimension of the first collection area is no more than 15, 12, 10, 8, 6, 5, 4, 3, 2, or no more than 1 cm.

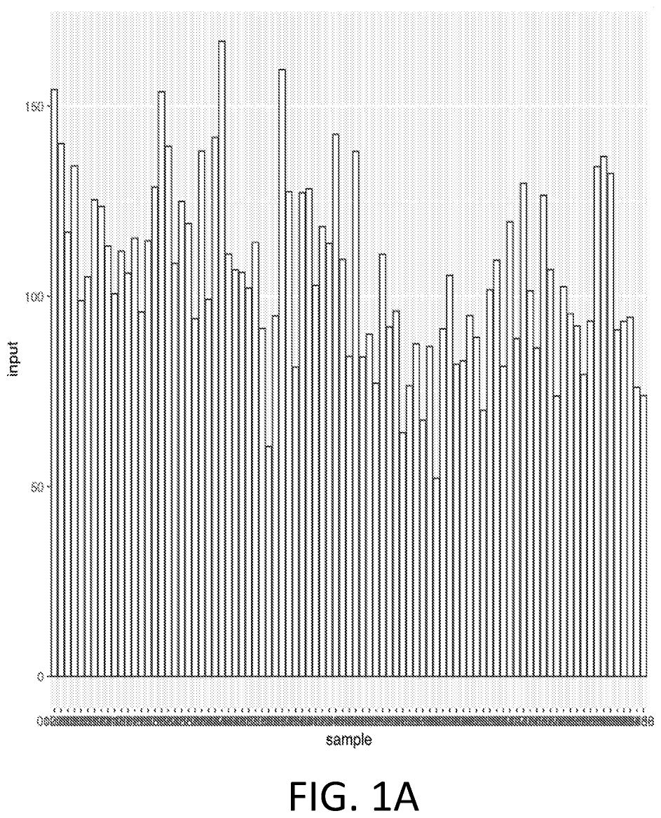

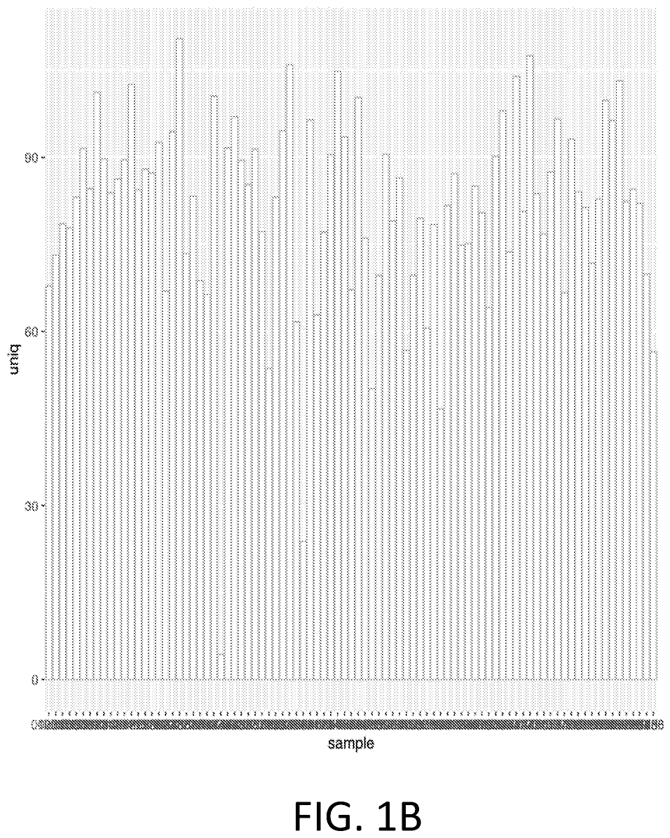

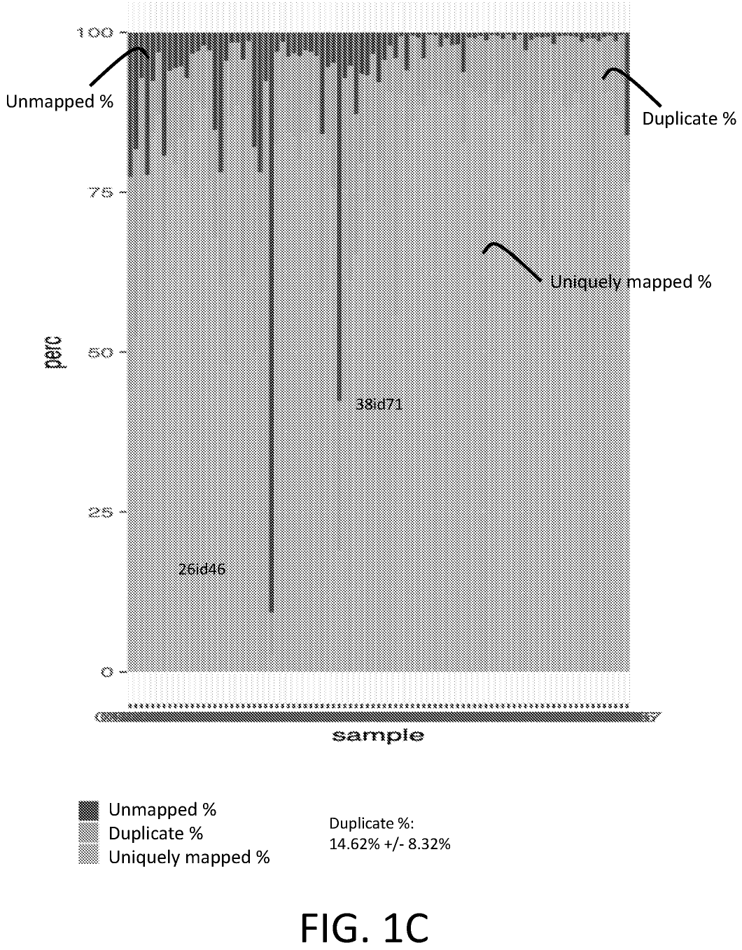

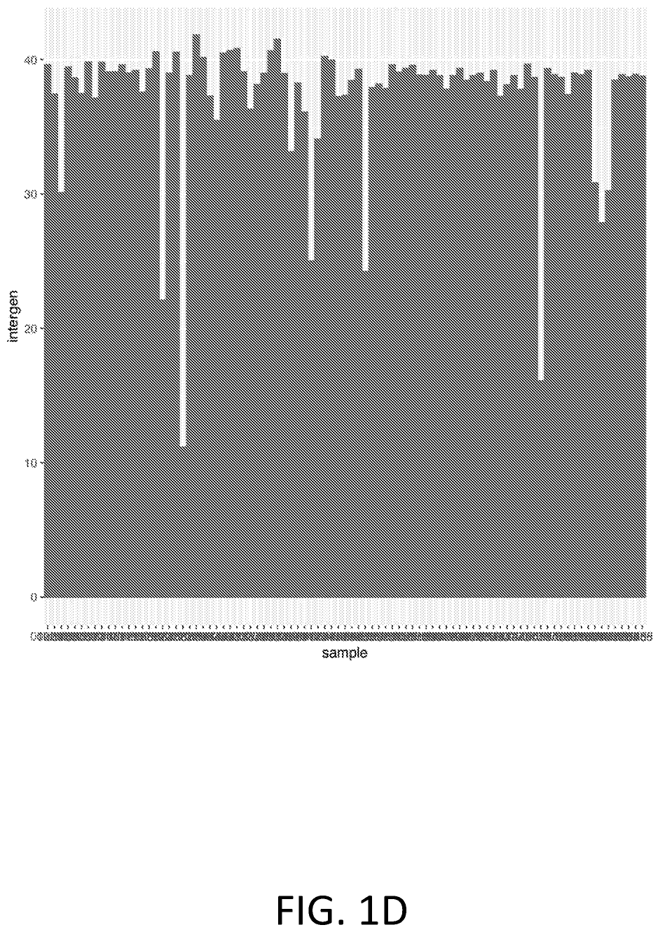

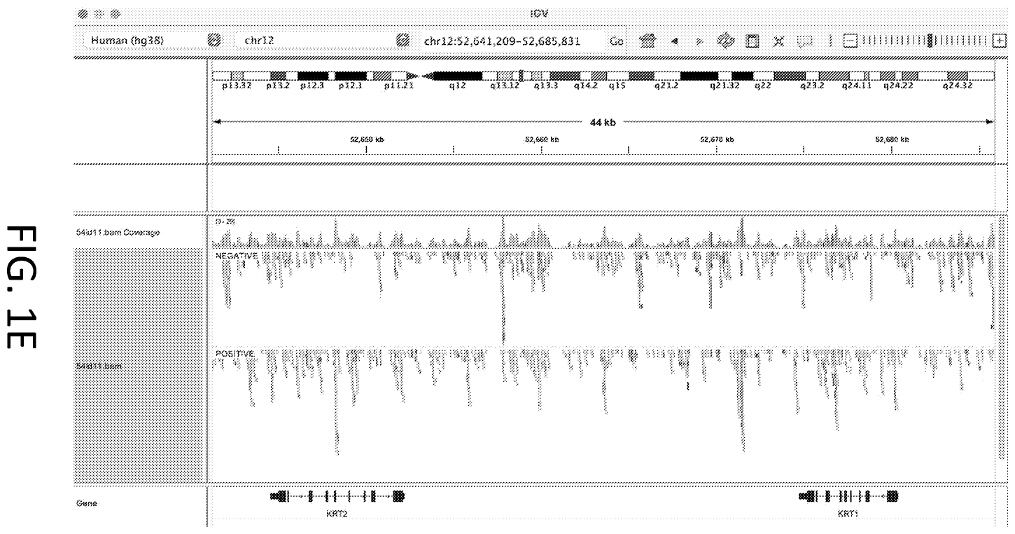

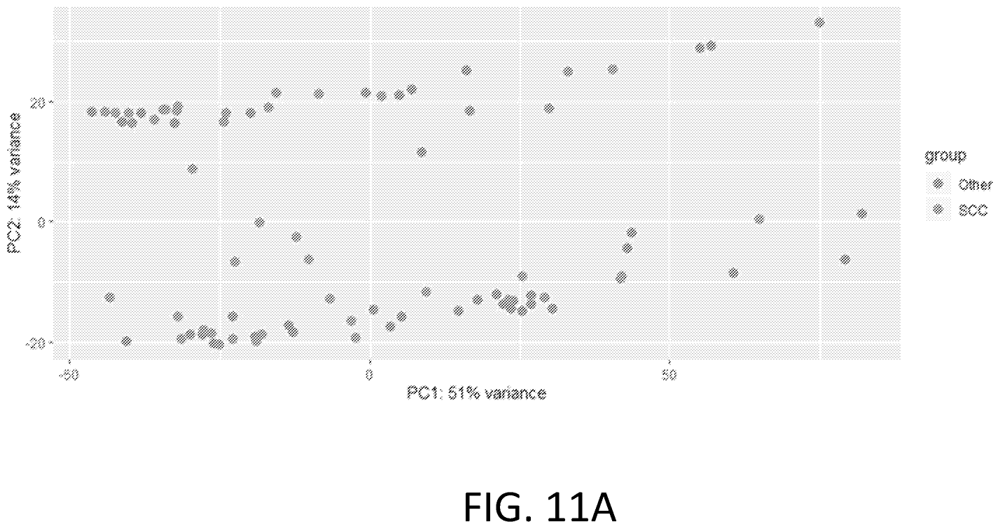

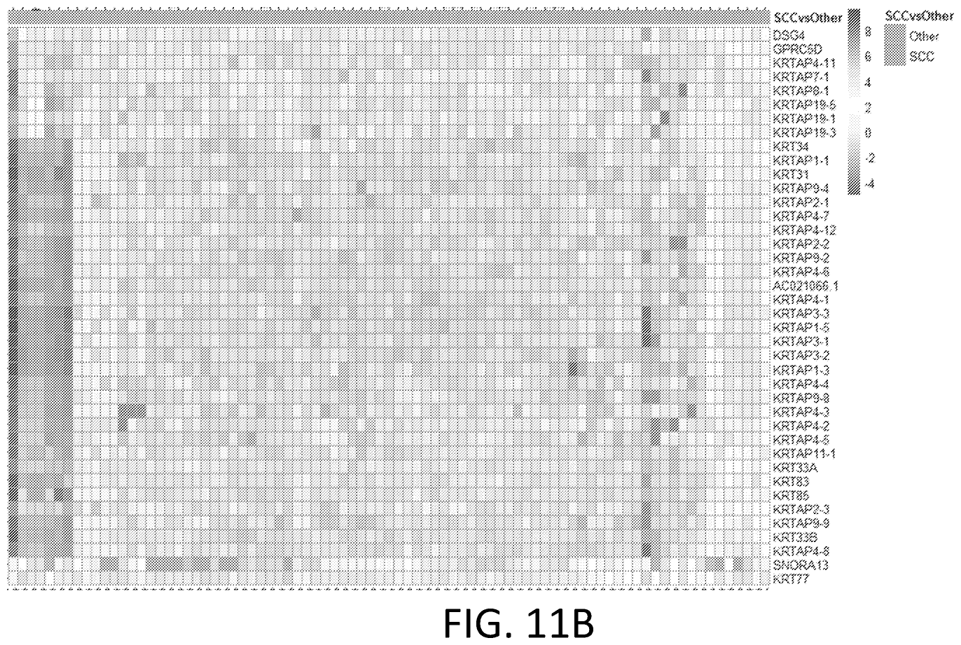

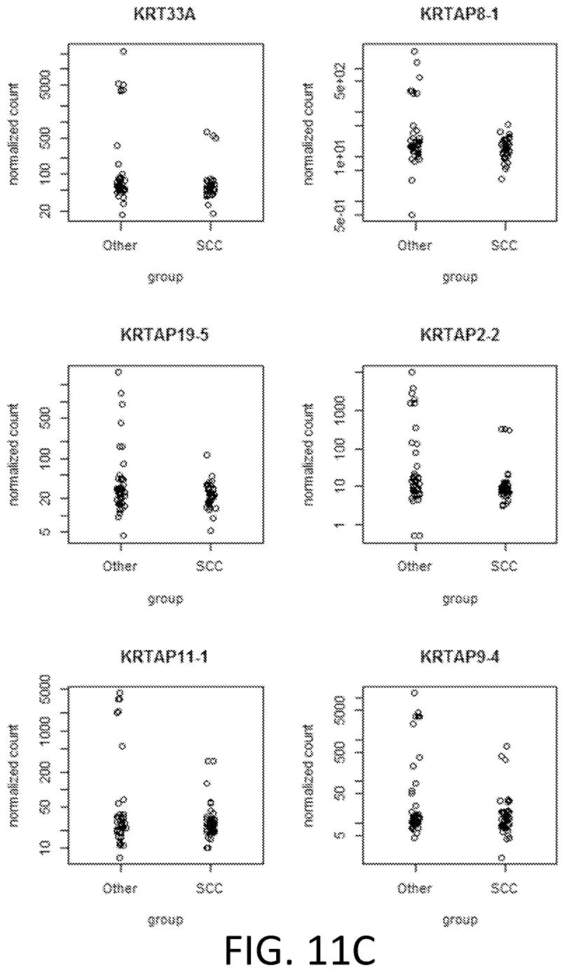

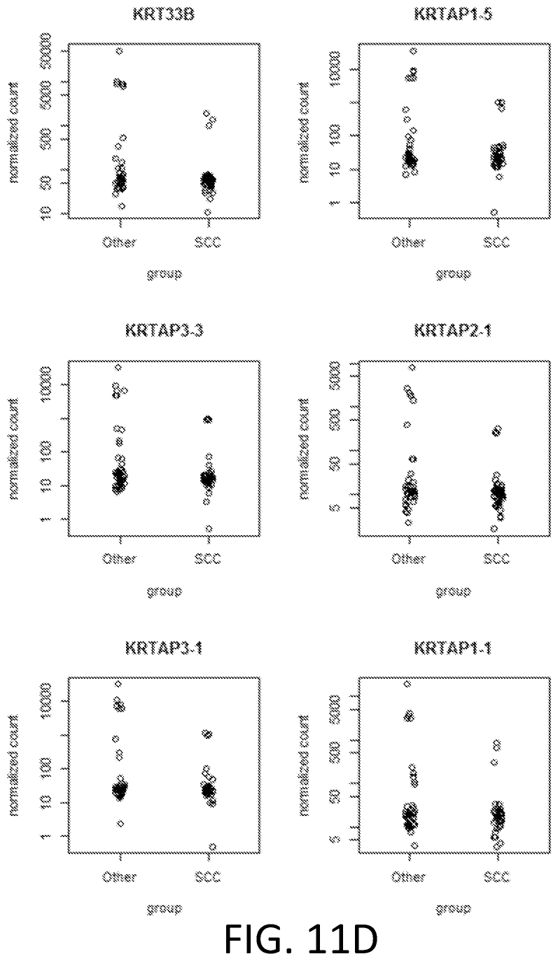

Patches may be configured for any size or shape. In some instances, patches are configured to adhere to specific areas of the body (e.g., face, head, or other area). In some instances, patches are configured as a single sheet covering the entire face. In some instances, multiple patches are configured to sample skin from the face. In some instances, patches are used as disclosed in FIGS. 11-13 of US 2016/0279401; or FIGS. 1-4 of US 20030167556, incorporated by reference in its entirety.

In some embodiments, a skin collection device such as an adhesive patch comprises a shape. The skin collection device may include 1 shape, or may include multiple shapes. A kit may include skin collection devices having separate shapes, for example 1, 2, 3, 4, 5, 6, 7, 8, 9, 10, or more different shaped collection devices. Examples of shapes include circles, ovals, squares, and the like. A shape may be straight. A shape may be generally composed of straight line segments. For example, the shape may include an angle (e.g. acute angle, obtuse angle, or right angle), balbis, concave polygon, constructible polygon, convex polygon, cyclic polygon, equiangular polygon, equilateral polygon, penrose tile, polyform, regular polygon, simple polygon, or tangential polygon. The shape may include a polygon with a specific number of sides, such as a triangle—3 sides, acute triangle, equilateral triangle, heptagonal triangle, isosceles triangle, golden triangle, obtuse triangle, rational triangle, right triangle, 30-60-90 triangle, isosceles right triangle, kepler triangle, scalene triangle, quadrilateral—4 sides, cyclic quadrilateral, kite, parallelogram, rhombus (equilateral parallelogram), lozenge, rhomboid, rectangle, square (regular quadrilateral), tangential quadrilateral, trapezoid, isosceles trapezoid, pentagon—5 sides, hexagon—6 sides, lemoine hexagon, heptagon—7 sides, octagon—8 sides, nonagon—9 sides, decagon—10 sides, hendecagon—11 sides, dodecagon—12 sides, tridecagon—13 sides, tetradecagon—14 sides, pentadecagon—15 sides, hexadecagon—16 sides, heptadecagon—17 sides, octadecagon—18 sides, enneadecagon—19 sides, icosagon—20 sides, star polygon—there are multiple types of stars, pentagram star polygon with 5 sides, hexagram—star polygon with 6 sides, star of David, heptagram—star polygon with 7 sides, octagram—star polygon with 8 sides, star of Lakshmi, enneagram—star polygon with 9 sides, decagram—star polygon with 10 sides, hendecagram—star polygon with 11 sides, dodecagram—star polygon with 12 sides, or apeirogon—generalized polygon with countably infinite set of sides. The shape may be curved. The shape may be composed of circular arcs. For example, the shape may include an annulus, arbelos, circle, Archimedes' twin circles, bankoff circle, circular triangle, reuleaux triangle, circumcircle, disc, incircle and excircles of a triangle, nine-point circle, circular sector, circular segment, crescent, lens, vesica piscis (fish bladder), lune, quatrefoil, reuleaux polygon, reuleaux triangle, salmon, semicircle, tomahawk, trefoil, triquetra, or heart shape. In some embodiments, the shape may not be composed of circular arcs. For example, the shape may include an Archimedean spiral, astroid, cardioid, deltoid, ellipse, heartagon, lemniscate, oval, cartesian oval, cassini oval, oval of booth, ovoid—similar to an oval, superellipse, taijitu, tomoe, or magatama shape.

The shape may be based on a skin collection area. For example, the skin collection device may include a single large patch, include face mask, be shaped for a forehead (e.g., be kidney shaped), be shaped to go under eyes (e.g. crescent), be shaped to cover at least part of a nose, be shaped to cover at least part of a right cheek, be shaped to cover at least part of a left cheek, may be postauricular, may be shaped to cover at least part of a right or left hand, or may be shaped to cover at least part of a right or left foot.

Parameters which effect the static wrinkling may include elasticity of the skin, elasticity of the backing layer, strength of the adhesive, and geometric parameters such as the length and width of the tape. One or more of these parameters may affect a wavelength and frequency of wrinkling patterns in the backing layer. Of these, the elasticity of the skin may not be readily controllable. For example, it may be a property of the skin to which the patch may adhere. An adhesive patch may comprise one or more of the following properties: a backing thickness greater than 3 mil, a longest dimension less than 10 cm, and a backing layer with an elastic modulus between 200 and 2000 PSI. An adhesive patch may comprise one or more of the following properties: a backing thickness greater than 3 mil, a longest dimension less than 5 cm, and a backing layer with an elastic modulus between 500 and 1500 PSI. An adhesive patch may comprise one or more of the following properties: a backing thickness greater than 3 mil, a longest dimension less than 5 cm, and a backing layer with an elastic modulus between 1000 and 2000 PSI. An adhesive patch may comprise an elastic modulus of from about 1000 to about 2000 Psi, about 500 to about 3000 Psi, about 250 to about 2000 Psi, about 400 to about 2000 Psi, about 500 to about 1500 Psi, about 750 to about 2000 Psi, about 1000 to about 3000 Psi, or about 500 to about 2500 Psi; a backing thickness greater than 3 mil; and a longest dimension less than 10 cm. An adhesive patch may comprise a longest dimension of about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm; a backing thickness greater than 3 mil; and a longest dimension less than 10 cm. An adhesive patch may comprise a backing thickness of about 3 mil, about 4 mil, about 5 mil, about 6 mil, about 7 mil, about 8 mil, about 9 mil, about 10 mil, about 20 mil, about 30 mil, about 40 mil, about 50 mil, about 60 mil, about 70 mil, about 80 mil, about 90 mil, about 100 mil, or about 125 mil; a longest dimension less than 10 cm; and a backing layer with an elastic modulus between 200 and 2000 PSI.

In further embodiments, the adhesive patch is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector. The peelable release sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The peelable release sheet is configured to hold about 12 adhesive patches. The peelable release sheet is configured to hold about 11 adhesive patches. The peelable release sheet is configured to hold about 10 adhesive patches. The peelable release sheet is configured to hold about 9 adhesive patches. The peelable release sheet is configured to hold about 8 adhesive patches. The peelable release sheet is configured to hold about 7 adhesive patches. The peelable release sheet is configured to hold about 6 adhesive patches. The peelable release sheet is configured to hold about 5 adhesive patches. The peelable release sheet is configured to hold about 4 adhesive patches. The peelable release sheet is configured to hold about 3 adhesive patches. The peelable release sheet is configured to hold about 2 adhesive patches. The peelable release sheet is configured to hold about 1 adhesive patch.

The adhesive patch is applied to the skin and removed from the skin. After removing the used adhesive patch from the skin surface, the patch stripping method further comprises storing the used patch on a placement area sheet, where the patch remains until the skin sample is isolated or otherwise utilized. The used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C. In some instances, patches are stored with a desiccant.

Skin collector kits may comprise an adhesive matrix. In some instances, the adhesive matrix comprises at least 3, 5, 8, 10, 11, 12, 13, 14, 15, 18, 20, or at least 25 oz/in² loop tackiness. In some instances, the adhesive matrix comprises 3-25, 3-20, 3-15, 5-20, 8-20, 10-15, 15-24, 10-20, or 1-20 oz/in² loop tackiness. In some instances, the adhesive matrix comprises a working temperature range from −40 to 176° F., −40 to 150° F., −40 to 130° F., −30 to 176° F., −20 to 176° F., −10 to 176° F., or −40 to 200° F. In some instances, the backing layer comprises at least 5, 8, 10, 12, 15, 18, 20, 23, 25, 30, or at least 55 lb/inch tensile force. In some instances, the backing layer comprises about 5, 8, 10, 12, 15, 18, 20, 23, 25, 30, or about 55 lb/inch tensile force. In some instances, the backing layer comprises 5-55, 5-40, 5-30, 5-25, 1-50, 10-20, 10-30, 15-30, 15-45, 20-45, 25-40, 30-50, or 25-60 lb/inch tensile force. In some instances, the backing layer comprises about 50, 80, 100, 120, 150, 180, 200, 230, 250, 300, 400, or about 500 mN tear strength. In some instances, the backing layer comprises 50-550, 50-400, 50-300, 50-250, 100-500, 100-200, 100-300, 150-300, 150-450, 200-450, 250-400, 300-500, or 250-600 mN tear strength.

In some instances, one or more components of the skin collector kit may be water soluble. In some instances, the adhesive patch is water soluble. In some instances, one or more of the backing layer and adhesive matrix are water soluble. In some instances, the placement area sheet is water soluble. In some instances, backing layer or adhesive matrix is configured to dissolve during skin sample lysis. In some instances, the adhesive patch is dissolvable in no more than 10, 15, 20, 30, 40, 50, 60, 90, or not more than 120 seconds. In some instances, the adhesive patch is dissolvable in no more than 10, 15, 20, 30, 40, 50, 60, 90, or not more than 120 seconds in an aqueous solution. In some instances, the adhesive patch is dissolvable in no more than 10, 15, 20, 30, 40, 50, 60, 90, or not more than 120 seconds in an aqueous solution having a temperature of no more than 30 degrees C. In some instances, the adhesive patch is dissolvable in no more than 10, 15, 20, 30, 40, 50, 60, 90, or not more than 120 seconds in an aqueous solution having a temperature of no more than 20 degrees C. In some instances, wherein the adhesive patch has shelf life of at least 1, 2, 3, 6, 8, 12, 14, 16, or at least 24 months. In some instances, wherein the adhesive patch has an shelf life of at least 1, 2, 3, 6, 8, 12, 14, 16, or at least 24 months at a temperature of no more than 30 degrees C.

An adhesive patch may comprise a water soluble adhesive. Current skin sample collection tools in some instances comprise non-invasive sample collection and genomic tests using an adhesive patch that comprises two parts: (a) a layer of non-water soluble adhesive (styrene-butadiene diblock copolymer) on (b) a thin backing sheet of thermoplastic polyurethane (TPU) film. This non-water soluble adhesive in some instances may cause the sample-loaded patches to stick together (self-fold or between patches) during sample lysis incubation, which prevents nucleic acids (DNA and RNA) releasing from the samples collected on patch to lysis solution (reduced the nucleic acid recovery yields) and the use of larger sample collection patch for sample collection (due to a higher incident of patch self-fold sticking in sample lysis incubation tube), in some instances limiting this non-invasive sample application tool to genomic test applications that may run on minute quantity of samples, and the incubation of multiple sample-loaded patches in one tube (due to sticking between patches, so each patch has to be incubated in a separate tube) may increase the cost on sample preparation for some genomic tests. The non-water soluble TPU backing sheet of the patch in some instances has disadvantages, e.g., the TPU film is removed from the lysis tube at the end of lysis incubation (before magnetic beads are added to the lysis tube), to prevent magnetic beads from sticking to the adhesive on the TPU film (those beads will get lost from the process, together with the sample nucleic acids bound on these beads). The process of removing TPU film in some instances presents challenges such as interrupting the workflow of the extraction process (increase in both labor work, time), a fully manual process that prevents process automation, increasing the chance of cross-contamination between samples, and potentially causing loss of nucleic acid-containing sample lysis solution that be trashed with the TPU films), reducing the sample nucleic acid yields. In some instances, use of water soluble adhesives and/or patches improves performance of the non-invasive sampling systems and methods described herein. Soluble adhesive patches are used to non-invasively collect skin samples for genomic testing, i.e., collecting skin samples with (water) soluble adhesive patches and these sample-loaded adhesive patches (including adhesive and backing sheet) will dissolve in the lysis solution with the collected skin samples during sample extraction. These soluble adhesive patches in some instances allow all sample-loaded patches to incubate in one lysis tube (reducing sample prep cost and time) and skipping the manual step of removing the backing films from lysis tubes. In some instances, use of soluble adhesive patches allow for automation of the sample process to save time and labor costs, and reduces the chance of cross-contamination and lost samples. In some instances, use of soluble adhesive patches provides increased utilization (up to 100%) of all collected skin tissues for nucleic acid extraction. In some instances, use of soluble adhesive patches provides at least 50%, 60%, 70%, 80%, 90%, 95%, or at least 99% utilization of all collected skin tissues for nucleic acid extraction. In some instances, all skin tissues on soluble patches are released to the lysis solution, compared to the non-soluble patches where some skin tissues still remain trapped in the non-soluble adhesive layers after lysis incubation.

Water soluble adhesives may generally include adhesives formed by copolymerization of a hydrophilic monomer with a monomer that used in an adhesive resin. Monomers used in adhesive resins may include monomers of one or more adhesive matrix materials described herein, for example, one or more of acrylics, silicones and hydrocarbon rubbers (like butyl rubber, styrene-butadiene rubber, ethyl-vinyl acetate polymers, styrene-isoprene-butadiene rubbers), or combination thereof. Monomers used in adhesive resins may include monomers of one or more adhesive matrix materials such as, for example, polyvinylpyrrolidone, polyacrylamide, polyacrylic acid, polyvinyl ethers, cellulose ethers, natural or synthetic gums, and polyethers (e.g., polyethylene glycol). Formulations of adhesive resins may include various types of water soluble and/or water dispersible salts, plasticizers, tackifiers, and surfactants. Tackifiers and plasticizers may be used to improve adhesion in formulations of adhesive resins. Example tackifiers and plasticizers may include one or more of, for example, ethoxylates, glucosides, rosins, and polyols.

Water soluble adhesives may generally include adhesives formed by conversion of an acrylic adhesive, which may not be sufficiently water soluble, to a more water soluble adhesive. Water solubility may be increased, for example, by neutralization of a carboxylic group in a pendant group of the monomer. The resultant polymer may, optionally, be plasticized with polyethylene glycol or polypropylene glycol. In an example, adhesive monomers such as, for example, butyl acrylate, acrylic acid, di-2-ethylhexyl fumarate, and/or vinyl acetate may be copolymerized, followed by the addition of an ethoxylated tert-N-alkyl diamine (an ethoxylated surfactant) as a plasticizer and/or tackifier and potassium hydroxide (neutralization agent). See for example, U.S. Pat. No. 3,441,430, which is incorporated herein by reference in its entirety.

Water soluble adhesives may generally include adhesives formed from acrylic acid and acrylamide, a polyhydric alcohol surfactant (tackifier/plasticizer), and a caustic (neutralization agent). See for example, U.S. Pat. No. 4,388,432, which is incorporated herein by reference in its entirety.

Water soluble adhesives may generally include adhesives formed from copolymers of acrylic acid and acrylates. These copolymers can be neutralized with aminopropanol followed by the addition of glycol ether. See, for example, JP Patent JP-56-7007 which is incorporated herein by reference in its entirety.

Water soluble adhesives may generally include adhesives formed from copolymers of 2-ethylhexyl acrylate, hydroxyethyl methacrylate, and acrylic acid. The copolymer may be neutralized with sodium hydroxide in methanol to make a water soluble adhesive. The formulation may include polyethylene glycol (tackifier/plasticizer) and polypropylene glycol diglycidyl ether (tackifier/plasticizer). See, for example, JP Patent JP-57-156456, which is incorporated herein by reference in its entirety.

Water soluble adhesives may generally include adhesives formed from polyethylene glycol, polypropylene glycol, or similar hydrophilic polymers or surfactants with hydroxyl or amine groups grafted to acrylic acid pendant groups on the adhesive polymers.

Water soluble adhesives may generally include adhesives formed from polyvinyl alcohol, cellulose ethers, and blends of such polymers. The adhesive formulation may be blended with water, dispersible/soluble additives, and other thermoplastics.

In some instances, the placement area sheet comprises a removable liner, provided that prior to storing the used patch on the placement area sheet, the removable liner is removed. The placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The placement area sheet is configured to hold about 12 adhesive patches. The placement area sheet is configured to hold about 11 adhesive patches. The placement area sheet is configured to hold about 10 adhesive patches. The placement area sheet is configured to hold about 9 adhesive patches. The placement area sheet is configured to hold about 8 adhesive patches. The placement area sheet is configured to hold about 7 adhesive patches. The placement area sheet is configured to hold about 6 adhesive patches. The placement area sheet is configured to hold about 5 adhesive patches. The placement area sheet is configured to hold about 4 adhesive patches. The placement area sheet is configured to hold about 3 adhesive patches. The placement area sheet is configured to hold about 2 adhesive patches. The placement area sheet is configured to hold about 1 adhesive patch.

The used patch is stored so that the matrix containing, skin facing surface of the used patch is in contact with the placement area sheet. In some instances, the placement area sheet is a panel of the tri-fold skin sample collector. In some instances, the tri-fold skin sample collector may further comprise a clear panel. The tri-fold skin sample collector may be labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. The patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances the placement area panel is a single placement area panel sheet. The identity of the skin sample collected is indexed to the tri-fold skin sample collector or placement area panel sheet by using a barcode or printing patient information on the collector or panel sheet. The indexed tri-fold skin sample collector or placement sheet is sent to a diagnostic lab for processing. The used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments the indexed tri-fold skin sample collector or placement sheet is sent to a diagnostic lab using UPS or FedEx.

In some embodiments, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit comprises at least one adhesive patch, a sample collector, and an instructions for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector may further comprise a barcode and/or an area for transcribing patient information. The adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provides the kit operator all of the necessary information for carrying out the patch stripping method. The instructions for use sheet preferably includes diagrams to illustrate the patch stripping method.

In some instances, the adhesive skin sample collection kit provides all the necessary components for performing the patch stripping method. In some instances, a kit comprises one or more of at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a packaging comprising instructions. In some embodiments, the adhesive skin sample collection kit includes a lab requisition form for providing patient information. In some instances, the kit further comprises accessory components. Accessory may components include, but are not limited to, a marker, a resealable plastic bag, gloves, and a cleansing reagent. The cleansing reagent includes, but is not limited to, an antiseptic such as isopropyl alcohol. In some instances, a skin sample collection kit may be provided in a cardboard box. In some instances, a kit comprises any of the skin collection components described herein. In some instances, a kit further comprises packaging comprising instructions. In some instances, the instructions are provided to perform one or more of the following: marking the patch to approximately a size of a lesion on a skin; peel the patch slowly; and peel at an angle greater than about perpendicular to the skin surface. In some instances, slowly is indicated as less than about 0.5, 0.7, 0.8. 0.9, 1, 1.1, 1.2, 1.5, 2.0, or 2.5 linear inches peeled per about five seconds. In some instances, slowly is indicated as less than about 0.5, 0.7, 0.8. 0.9, 1, 1.1, 1.2, 1.5, 2.0, or 2.5 linear inches peeled per about ten seconds. In some instances, slowly is indicated as less than about 0.5, 0.7, 0.8. 0.9, 1, 1.1, 1.2, 1.5, 2.0, or 2.5 linear inches peeled per about three seconds.

A kit described herein may comprise a means for preservation or storage of a collected skin sample. In some instances, a kit for non-invasive collection and analysis of a skin sample comprises at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a return pouch sized and shaped to receive the at least one adhesive patch, the return pouch comprising a preservative. In some instances, the preservative is a desiccant. In some instances, the preservative is configured to prevent degradation of biological molecules sampled using the collector kit. In some instances, the desiccant is configured to prevent the activity of nucleases in the skin sample. In some instances, the desiccant is configured to prevent degradation of nucleic acids in the sample. In some instances, the desiccant is configured to prevent the activity of RNases, DNAases, or both RNAases and DNAses in the skin sample. In some instances, the amount of the desiccant is from about 0.5 grams to about 5 grams, about 0.1 grams to about 10 grams, about 0.1 grams to about 5 grams, about 0.5 grams to about 5 grams, about 0.1, 0.5, 1, 1.5, 2.0, 2.5, 3, 3.5, 4, 4.5, 5, 5.5, 6, 8, 10, 12, 15 or about 20 grams. In some instances, the kit comprises a return pouch. In some instances, the return pouch is plastic or foil. In some instances, the return pouch is sealable. In some instances, the desiccant is silica gel. In some instances, a skin collection kit does not comprise a desiccant.

In some embodiments, the skin sample is obtained from a skin lesion. In some cases, the skin lesion is a pigmented skin lesion comprising a mole, dark colored skin spot, or melanin containing skin area. In some cases, the skin lesion is an area on the skin surface that is suspicious for melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, merkel cell carcinoma, sebaceous carcinoma, squamous cell carcinoma, and dermatofibrosarcoma protuberans. In some instances, the skin lesion is suspicious for skin cancer. Exemplary skin cancer include, but are not limited to, melanoma, basal cell carcinoma (BCC), squamous cell carcinoma (SCC), angiosarcoma, cutaneous B-cell lymphoma, cutaneous T-cell lymphoma, dermatofibrosarcoma protuberans, Merkel cell carcinoma, and sebaceous gland carcinoma. In some instances, the skin lesion is suspicious for melanoma. In some instances, the skin lesion is suspicious for basal cell carcinoma. In some instances, the skin lesion is suspicious for squamous cell carcinoma.

In some cases, the skin lesion is from about 5 mm to about 20 mm in diameter.

Methods and compositions as described herein, in certain embodiments, result in obtaining various layers of skin. In some instances, the layers of skin include epidermis or dermis. The epidermis is further subdivided into stratum corneum, stratum lucidum, stratum granulosum, stratum spinosum, and stratum germinativum. In some instances, the skin sample is obtained from the epidermis layer, optionally from one or more of stratum corneum, stratum lucidum, stratum granulosum, stratum spinosum, and stratum germinativum. In some instances, the skin sample is obtained from the dermis layer. In some instances, cells are obtained from the skin using methods and compositions as described herein. Exemplary cells that are obtained include, but are not limited to, keratinocytes, melanocytes, basal cells, T-cells, Merkel cells, Langerhans cells, fibroblasts, macrophages, adipocytes, and dendritic cells.

Provided herein are methods and compositions for extraction of nucleic acids from a biological sample such as a sample collected using an adhesive patch. In some instances, nucleic acids are extracted using any technique that does not interfere with subsequent analysis. In some instances, this technique uses alcohol precipitation using ethanol, methanol or isopropyl alcohol. In some instances, this technique uses phenol, chloroform, or any combination thereof. In some instances, this technique uses cesium chloride. In some instances, this technique uses sodium, potassium or ammonium acetate or any other salt commonly used to precipitate the nucleic acids.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

Following extraction of nucleic acids from a biological sample, the nucleic acids, in some instances, are further purified. In some instances, the nucleic acids are RNA. In some instances, the nucleic acids are DNA. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods and compositions for isolating or enriching nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 um, 1 um, 1.5 um, 2 um, 2.5 um, 3 um, 3.5 um, 4 um, 4.5 um, 5 um, 5.5 um, 6 um, 6.5 um, 7 um, 7.5 um, 8 um, 8.5 um, 9 um, 9.5 um, 10 um, or more than 10 um.

In some cases, a yield of the nucleic acids products obtained using methods described herein is about 500 picogram or higher, about 1000 picogram or higher, about 2000 picogram or higher, about 3000 picogram or higher, about 4000 picogram or higher, about 5000 picogram or higher, about 6000 picogram or higher, about 7000 picogram or higher, about 8000 picogram or higher, about 9000 picogram or higher, about 10000 picogram or higher, about 20000 picogram or higher, about 30000 picogram or higher, about 40000 picogram or higher, about 50000 picogram or higher, about 60000 picogram or higher, about 70000 picogram or higher, about 80000 picogram or higher, about 90000 picogram or higher, or about 100000 picogram or higher.

In some cases, methods described herein provide less than less than 10%, less than 8%, less than 5%, less than 2%, less than 1%, or less than 0.5% product yield variations between samples.

In some cases, methods described herein provide a substantially homogenous population of a nucleic acid product.

In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods and compositions for detecting an expression level of one or more genes of interest from nucleic acids isolated from a biological sample. In some instances, the expression level is detected following an amplification reaction. In some instances, the nucleic acids are RNA. In some instances, the expression level is determined using PCR. In some instances, the expression level is determined using qPCR. In some instances, primers and probes for use in the qPCR are specific to IVNS1ABP, RAB5B, CAPZA2, STK4, SCAF11, NCOA1, CBL, EFCAB2, KRTAP4-6, KRTAP9-4, U3, DDX3X, CYFIP1, ITGA3, MAGED1, SPRR2AM, MT-CO2, RNA5SP202, MIR4421, HAL, RNA5SP263, TIMP1, RNA5SP481, or a combination thereof. In some instances, primers and probes for use in the qPCR are specific to MT-ND3, JPH3, TIMP2, MT-ND4, SOX11, MED16, KIF1B, MT-ND1, COL17A1, HERC4, AKR1C1, TAGLN, SLCO1A2, ZIC2, LINC02167, ATPAF2, PUM2, CYP24A1, CCT8, MT-CYB, WARS, METTL9, FOXI3, EDN1, MUC5B, WIP11, HLA-H, CDV3, FMNL3, STRA6, CASC15, FSTL1, ATG4B, KRT7, FDCSP, EAF1, SLC5A10, KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1. In some instances, primers and probes for use in the qPCR are specific to TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, GBP1, WARS, SLCO1A2, ATPAF2, MT-ND1, ATG4B, MUC5B, WIP11, HERC4, PUM2, COL17A, METTL9, EAF1, CDV3, TIMP2, ZIC2, AKR1C1, HLA-H, MT-CYB, SLC5A10, KRT7, or a combination thereof. In some instances, primers and probes for use in the qPCR are specific MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1 or a combination thereof. In some instances, primers and probes for use in the qPCR are specific MT-ND3, JPH3, TIMP2, MT-ND4, SOX11, and MED16 or a combination thereof. In some instances, primers and probes for use in the qPCR are specific KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1 or a combination thereof. In some instances, primers and probes for use in the qPCR are specific DSG4, GPRC5D, KRTAP4-11, KRTAP7-1, KRTAP8-1, KRTAP19-5, KRTAP19-1, KRTAP19-3, KRT34, KRTAP1-1, KRT31, KRTAP9-4, KRTAP2-1, KRTAP4-7, KRTAP4-12, KRTAP2-2, KRTAP9-2, KRTAP4-6, AC021066.1, KRTAP4-1, KRTAP3-3, KRTAP1-5, KRTAP3-1, KRTAP3-2, KRTAP1-3, KRTAP4-4, KRTAP9-8, KRTAP4-3, KRTAP4-2, KRTAP4-5, KRTAP11-1, KRT33A, KRT83, KRT85, KRTAP2-3, KRTAP9-9, KRT33B, KRTAP4-8, SNORA13, and KRT77 or a combination thereof. In some instances, primers and probes for use in the qPCR are specific TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1 or a combination thereof. In some instances, primers and probes for use in the qPCR are specific TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1 or a combination thereof. In some instances, primers and probes for use in the qPCR are specific KRT85, TIMP1, FP236383.5, LCE6A, LCE2C, LCE1F, FP236383.4, IL37, LCE5A, AL139247.1, WARS1, and GBP1 or a combination thereof. In some instances, primers and probes for use in the qPCR are specific KRT85, KRT83, FP671120.7, FP236383.4, FP236383.5, LCE1F, LCE1B, LCE1C, LCE6A, LCE2D, LCE2A, LCE2C, LCE2B, AL139247.1, HPGD, KCTD4, IL37, LCE5A, RNU1-3, WARS1, GBP1, AC104109.2, C2orf76, PSG7, PLCXD2, KRT28, RNF5, IGF2BP2, TIMP1, EDN1, and KRT16P6 or a combination thereof. In some instances, the expression level is determined using a microarray. In some instances, the expression level is determined by sequencing.

Provided herein are methods and compositions for detecting a mutational change of one or more genes of interest from nucleic acids isolated from a biological sample. In some instances, the mutational change is detected following an amplification reaction. In some instances, the nucleic acids are RNA. In some instances, the nucleic acids are DNA. In some instances, the mutational change is detected using allele specific PCR. In some instances, the mutational change is detected using sequencing. In some instances, the sequencing is performed using the Sanger sequencing method. In some instances, the sequencing involves the use of chain terminating dideoxynucleotides. In some instances, the sequencing involves gel-electrophoresis. In some instances, the sequencing is performed using a next generation sequencing method. In some instances, sequencing includes, but not limited to, single-molecule real-time (SMRT) sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, +S sequencing, and sequencing by synthesis.

Provided herein are systems and methods of sample processing for analysis of non-melanoma skin diseases (NMSC, e.g., detection of SCC, BCC, or SCC subtype, e.g., isSCC, ivSCC) in a subject or patient. In embodiments, systems and methods comprise measuring gene expression levels found in a sample from a patient. In some embodiments, systems and methods comprise identifying a subject with a skin disease. In some embodiments, systems and methods comprise diagnosing the patient with the skin disease. In some embodiments, systems and methods comprise treating the patient with the skin disease. In some embodiments, the treating comprises one or more treatments for SCC or BCC. In some embodiments, a treatment comprises one or more of excisional surgery, Mohs surgery, cryosurgery, curettage and electrodesiccation (electrosurgery), laser surgery, radiation, immunotherapy, photodynamic therapy (pdt), and applying topical medications. In some instances, topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. In some embodiments, immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

### Tissue Sampling and Cellular Material

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids and proteins. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA includes, but not limited to, picogram, nanogram, and microgram quantities.

In still further or additional embodiments, the adhered skin sample comprises cellular material including nucleic acids such as RNA or DNA, or a polypeptide such as a protein, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In further or additional embodiments, the amount of cellular material, including nucleic acids such as RNA or DNA, or a polypeptide such as a protein, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR. In some instances, a one-step RT-PCR method is utilized. In some III instances a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as skin cancer. In some embodiments, a software program or module scores a sample based on gene expression levels. In some embodiments, the sample score is compared with a reference sample score to determine if there is a statistical significance between the gene expression signature and a disease.

Sample collection (e.g., patch stripping) can be performed using one or more components provided herein in an adhesive skin sample collection kit. The patch stripping method in some instances comprises applying and removing an adhesive patch to the skin surface of a subject. The adhesive patch comprises an adhesive matrix, wherein during application of the adhesive patch to the skin surface, an effective amount of a skin sample containing cellular material adheres to the adhesive matrix. The adhered skin sample is retained on the adhesive matrix upon removal of the patch from the skin surface. The adhesive patch containing the adhered skin sample is designated as a used adhesive patch. The adhesive patch is configured so that at least a portion of the skin sample cellular material can be harvested from a used patch.

In some embodiments, the method of collection of cellular or other material on the skin comprises using from one, two, three, four, five, six, seven, eight, nine, 10, 11, 12, 13, 14, 15, 16, 17, 18, 19, 20, or more patches in methods described herein.

The adhesive skin sample collection kit for use with patch stripping methods is provided as a non-invasive means to collect skin samples with minimal discomfort. Cellular material is isolated from the skin sample and can be utilized in tests that can determine the stage of disease, the risk of disease progression and a patient's likelihood of responding to a particular treatment. Treatments include drug therapies and biopsy. Skin sample cellular materials include nucleic acids, polypeptides, lipids, carbohydrates and small molecules. Nucleic acids include DNA and RNA.

Provided herein are methods of extracting biomolecules from skin samples. In some instances, biomolecules are extracted from patches described herein. In some instances, biomolecules comprise nucleic acids. In some instances, extraction of nucleic acids comprises one or more of lysing, binding, washing and elution of nucleic acids attached to patches. In some instances, samples are first lysed, which involves breaking the cell membrane and freeing the nucleic acid. Ethanol is added to the lysate to provide ideal binding conditions. The binding solution is then loaded onto the RNeasy silica spin column membrane. The wash buffers are added to the column and centrifuged three times to force the buffer through the column and wash away any remaining impurities from the membrane, leaving RNA bound to the silica gel. The elution buffer (water) is added to the column and centrifuged to remove the nucleic acid from the membrane and the nucleic acid is collected from the bottom of the column.

Adhesive patches may be configured to minimize extractables (or leachables), which in some instances may lead to interference with nucleic acid experiments. In some instances, an extractable or a leachable comprises a component of the system that is not the skin sample. Interference (volatile residuals, additives, fillers, binders, etc.) with RT-PCR test that could be present in alternative or prototype patches for this skin stripping application in some instances is analyzed by GC-MS extraction of patch samples using solvents such as ethanol and isopropanol (which are used for RNA isolation) and quantified via the standard curve method with known concentrations of standard solutions. In some instances, methods may reflect the disclosure of WO 2018/191268, the entire disclosure of which is incorporated herein by reference. In some instances, the method comprises one or more steps of: a) contacting the biological sample obtained from an individual in need thereof with a plurality of beads; b) co-isolating RNA and genomic DNA from the plurality of beads; c) amplifying both the RNA and genomic DNA extracted from step (b); d) detecting the expression level of a RNA of interest from the RNA isolated from the beads; and e) detecting a mutational change, a methylation status, or a combination thereof from a gene of interest from the genomic DNA isolated from the beads. In some instances, this classification allows the quality of each patch with respect to the unnecessary extractables released to the analysis solution. In some instances, extractables are measured from a patch comprising an adhesive matrix. In some instances, the extractables from a patch is about 70, 65, 60, 55, 50, 45, 40, 35, 30, 25, 20, 15, 10, or about 5 ppm per 25 cm² (3.875 square inches) area using a 20:80 IPA:H₂O extraction medium. In some instances, the extractables from a patch is no more than 70, 65, 60, 55, 50, 45, 40, 35, 30, 25, 20, 15, 10, or no more than 5 ppm per 25 cm² (3.875 square inches) area using a 20:80 IPA:H₂O extraction medium. In some instances, the extractables from a patch is about 700, 650, 600, 550, 500, 450, 400, 350, 300, 250, 200, 150, 100, or about 50 ppm per 25 cm² (3.875 square inches) area using an 80:20 IPA:H₂O extraction medium. In some instances, the extractables from a patch is no more than 700, 650, 600, 550, 500, 450, 400, 350, 300, 250, 200, 150, 100, or no more than 50 ppm per 25 cm² (3.875 square inches) area using an 80:20 IPA:H₂O extraction medium. In some instances, the patches do not comprise a substantial amount of volatile (e.g. unreacted monomers), semi-volatile (e.g. plasticizers, process aids) or ash (e.g. inorganic fillers) type ingredients as analyzed by TGA (Thermogravimetric Analysis) TA Q50 TGA instrument. In some instances, TGA data is analyzed using TA Universal Analysis Software (no significantly measurable fillers, binders or catalysts). In some instances, unreacted monomers, semi-volatile (e.g. plasticizers, process aids) or ash (e.g. inorganic fillers) levels are below a detection limit of about 50, 40, 30, 25, 20, 18, 15, 13, 10, 8, 6, 5, or 3 ug/L of GC-MS. In some instances, BHT (butylated hydroxytoluene) levels are below a detection limit of about 50, 40, 30, 25, 20, 18, 15, 13, 10, 8, 6, 5, or 3 ug/L of GC-MS. In some instances, an amount of extractables and leachables released from the at least one adhesive patch is no more than 1.0, 1.5, 2.0., 2.5, 3.0, 3.5, 4.0, 4.5, 5.0, 5.5, or no more than 6.0, mg/cm² when at least about 25 cm² of adhesive patch is refluxed for about 3 hours in 80% ethanol.

In some embodiments, isolated RNA from a collected skin sample is reverse transcribed into cDNA for amplification by PCR to enrich for target genes. The expression levels of these target genes are quantified by quantitative PCR in a gene expression test. A gene expression test provides information on a gene expression signature associated with a disease. A pigmented lesion assay is an exemplary gene expression test which measures the expression levels of target genes from RNA isolated using the adhesive skin sample collection kit.

For example, in some embodiments, the pigmented lesion assay provides objective information on a gene expression signature associated with melanoma. This information can be used to help support a histopathologic diagnosis or to determine the need for a biopsy, thereby reducing unnecessary biopsy procedures. The development of invasive tumor properties is also controlled by gene expression; therefore, the pigmented lesion assay may also differentiate invasive melanoma from melanoma in situ as well as provide staging information. The identification of invasive melanoma with metastatic potential will direct treatments to only those who need it. Another gene expression assay may determine if a melanoma tumor has spread to the lymph nodes. This test can reduce the need for a sentinel lymph node surgery, which can be extensive, cause morbidity and has significant medical costs.

Gene expression analyses facilitate drug development by identifying drug targets and stratifying patients into groups that will maximize a drug response. In an exemplary embodiment, a skin sample collected from the face of a subject with lupus is isolated and utilized in a gene expression test to assess the expression of target genes indicated in lupus drug effects. This gene expression test can identify responders to therapy and identify new drug targets. The use of the adhesive patch allows for skin sample collection without the scarring that can occur with a biopsy.

In some embodiments, one or more polypeptides isolated from the used adhesive patch are detected and/or quantified. For example, in some embodiments, one or more polypeptides isolated from the used adhesive patch are detected and/or quantified using ELISA, immunohistochemistry, mass spectrometry, and/or absorbance measurement. In some embodiments, the sequence of DNA isolated from the used adhesive patch is determined using gene sequencing methods known to one of skill in the art.

In some instances, the skin sample collected using the patch stripping method is used in combination with other clinical assays including immunohistochemistry, immunophenotyping, fluorescent in situ hybridization (FISH), and/or any combination thereof. The skin sample does not necessarily need to be removed from the adhesive patch to prove useful as an assay component. Cellular material from the skin samples can be detected from the surface of the adhesive patch matrix. Detection methods include the use of probes configured to bind to cellular material adhered to the adhesive patch matrix. Probes include, but are not limited to, primers configured to bind to nucleic acids, and antibodies configured to bind to polypeptides, nucleic acids, small molecules, lipids, and/or carbohydrates.

In some embodiments, the patch stripping method is part of the work up for a variety of suspected skin conditions including, but not limited to, lupus, rubeola, acne, hemangioma, psoriasis, eczema, candidiasis, impetigo, shingles, leprosy and Chron's disease. Skin conditions also include inflammatory dermatoses, bullous diseases, infections, and cancers. Skin cancers include, but are not limited to, basal cell carcinoma, actinic keratoses, merkel cell carcinoma, sebaceous carcinoma, squamous cell carcinoma, melanoma, and dermatofibrosarcoma protuberans.

In some embodiments, the patch stripping method is performed using a plurality of adhesive patches. Between 1 and 8 adhesive patches can be sequentially applied and removed to collect a skin sample. The number of adhesive patches used per skin sample may include, but is not limited to, 12, 11, 10, 9, 8, 7, 6, 5, 4, 3, 2, 1, from about 2 to about 7, from about 3 to about 6, and from about 4 to about 5. In certain instances, an adhesive patch is applied to the skin and removed from the skin about 1 to about 8 times.

The methods, devices, and systems provided herein involve applying an adhesive or other similar patch to the skin in a manner so that an effective or sufficient amount of a tissue, such as a skin sample, adheres to the adhesive matrix of the adhesive patch. For example, in some embodiments, the effective or sufficient amount of a skin sample is an amount that removably adheres to a material, such as the matrix or adhesive patch. The adhered skin sample, in certain embodiments, comprises cellular material including nucleic acids and proteins. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA include picogram, nanogram, and microgram quantities. In some instances, the amount of cellular material or nucleic acids is measured per kit, per patch (or patch), or as a function of the surface area of the adhesive area of the patch.

The amount of cellular material collected may be measured per collection kit. In some instances, a collection kit comprises one or more patches. In some embodiments, the adhered skin sample comprises cellular material including nucleic acids such as RNA or DNA, or a polypeptide such as a protein, in an amount that is at least about 1 picogram per collection kit. In some embodiments, the amount of cellular material is no more than about 1 nanogram per collection kit. In further or additional embodiments, the amount of cellular material is no more than about 1 microgram per collection kit. In still further or additional embodiments, the amount of cellular material is no more than about 1 gram per collection kit. In further or additional embodiments, the amount of cellular material, including nucleic acids such as RNA or DNA, or a polypeptide such as a protein, is less than about 1 gram, is less than about 500 milligrams, is less than about 490 milligrams, is less than about 480 milligrams, is less than about 470 milligrams, is less than about 460 milligrams, is less than about 450 milligrams, is less than about 440 milligrams, is less than about 430 milligrams, is less than about 420 milligrams, is less than about 410 milligrams, is less than about 400 milligrams, is less than about 390 milligrams, is less than about 380 milligrams, is less than about 370 milligrams, is less than about 360 milligrams, is less than about 350 milligrams, is less than about 340 milligrams, is less than about 330 milligrams, is less than about 320 milligrams, is less than about 310 milligrams, is less than about 300 milligrams, is less than about 290 milligrams, is less than about 280 milligrams, is less than about 270 milligrams, is less than about 260 milligrams, is less than about 250 milligrams, is less than about 240 milligrams, is less than about 230 milligrams, is less than about 220 milligrams, is less than about 210 milligrams, is less than about 200 milligrams, is less than about 190 milligrams, is less than about 180 milligrams, is less than about 170 milligrams, is less than about 160 milligrams, is less than about 150 milligrams, is less than about 140 milligrams, is less than about 130 milligrams, is less than about 120 milligrams, is less than about 110 milligrams, is less than about 100 milligrams, is less than about 90 milligrams, is less than about 80 milligrams, is less than about 70 milligrams, is less than about 60 milligrams, is less than about 50 milligrams, is less than about 40 milligrams, is less than about 30 milligrams, is less than about 20 milligrams, is less than about 10 milligrams, is less than about 5 milligrams, or is less than about 1 milligram. In further or additional embodiments, the amount of cellular material is from about 1 picogram to about 1 gram per collection kit. In further or additional embodiments, the amount of cellular material is from about 1 picogram to about 1 milligram per collection kit. In further or additional embodiments, the amount of cellular material is from about 1 picogram to about 1 microgram per collection kit. In further or additional embodiments, the amount of cellular material is from about 1 picogram to about 1 nanogram per collection kit. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms per collection kit. In further or additional embodiments, the amount of cellular material, including nucleic acids such as RNA or DNA, or a polypeptide such as a protein, comprises an amount that is from about 50 microgram to 1 milligram, 50 microgram to 50 milligrams, 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms per collection kit. In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram per collection kit. In further or additional embodiments, the amount of cellular material is from about 1 picogram to about 50 microgram per collection kit. In further or additional embodiments, the cellular material comprises an amount that is from about 1 picogram to about 15 micrograms, about 1 picogram to about 10 micrograms, about 1 picogram to about 50 micrograms, about 1 picogram to about 50 micrograms, about 1 picogram to about 100 micrograms, about 1 picogram to about 200 micrograms, about 1 picogram to about 500 micrograms, about 1 picogram to about 750 micrograms, 50 picogram to about 1 microgram, from about 500 picogram to 1 microgram, from about 100 picograms to about 500 microgram, from about 500 picograms to about 100 microgram, from about 750 picograms to about 1 microgram, from about 1 nanogram to about 750 microgram, from about 100 nanogram to about 500 microgram, or from about 1 nanogram to about 500 microgram per collection kit. In further or additional embodiments, the amount of cellular material is from about 1 picogram to about 1 gram per collection kit. In further or additional embodiments, the cellular material comprises an amount that is from about 50 microgram to about 1 milligram, from about 500 microgram to 1 milligram, from about 100 picograms to about 500 milligram, from about 500 picograms to about 100 milligram, from about 750 picograms to about 1 milligram, from about 1 nanogram to about 750 milligram, or from about 1 nanogram to about 500 milligram per collection kit.

The amount of cellular material collected may be measured per area of the adhesive region of a patch. In some embodiments, the adhered skin sample comprises cellular material including nucleic acids such as RNA or DNA, or a polypeptide such as a protein, in an amount that is at least about 1 picogram per square inch. In some embodiments, the amount of cellular material is no more than about 1 nanogram per square inch. In further or additional embodiments, the amount of cellular material is no more than about 1 microgram per square inch. In still further or additional embodiments, the amount of cellular material is no more than about 1 gram per square inch. In further or additional embodiments, the amount of cellular material is from about 1 picogram to about 1 gram per square inch. In further or additional embodiments, the amount of cellular material is from about 1 picogram to about 1 milligram per square inch. In further or additional embodiments, the amount of cellular material is from about 1 picogram to about 1 microgram per square inch. In further or additional embodiments, the amount of cellular material is from about 1 picogram to about 1 nanogram per square inch. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms per square inch. In further or additional embodiments, the amount of cellular material, including nucleic acids such as RNA or DNA, or a polypeptide such as a protein, comprises an amount that is from about 50 microgram to 1 milligram, 50 microgram to 50 milligrams, 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms per square inch. In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram per square inch. In further or additional embodiments, the amount of cellular material is from about 1 picogram to about 50 microgram per square inch. In further or additional embodiments, the cellular material comprises an amount that is from about 1 picogram to about 15 micrograms, about 1 picogram to about 10 micrograms, about 1 picogram to about 50 micrograms, about 1 picogram to about 50 micrograms, about 1 picogram to about 100 micrograms, about 1 picogram to about 200 micrograms, about 1 picogram to about 500 micrograms, about 1 picogram to about 750 micrograms, 50 picogram to about 1 microgram, from about 500 picogram to 1 microgram, from about 100 picograms to about 500 microgram, from about 500 picograms to about 100 microgram, from about 750 picograms to about 1 microgram, from about 1 nanogram to about 750 microgram, from about 100 nanogram to about 500 microgram, or from about 1 nanogram to about 500 microgram per square inch. In further or additional embodiments, the amount of cellular material is from about 1 picogram to about 1 gram per square inch. In further or additional embodiments, the cellular material comprises an amount that is from about 50 microgram to about 1 milligram, from about 500 microgram to 1 milligram, from about 100 picograms to about 500 milligram, from about 500 picograms to about 100 milligram, from about 750 picograms to about 1 milligram, from about 1 nanogram to about 750 milligram, or from about 1 nanogram to about 500 milligram per square inch.

The amount of cellular material collected may be measured per patch per patch. In some instances, a kit comprises one or more patches. In some embodiments, the adhered skin sample comprises cellular material including nucleic acids such as RNA or DNA, or a polypeptide such as a protein, in an amount that is at least about 1 picogram per patch. In some embodiments, the amount of cellular material is no more than about 1 nanogram per patch. In further or additional embodiments, the amount of cellular material is no more than about 1 microgram per patch. In still further or additional embodiments, the amount of cellular material is no more than about 1 gram per patch. In further or additional embodiments, the amount of cellular material is from about 1 picogram to about 1 gram per patch. In further or additional embodiments, the amount of cellular material is from about 1 picogram to about 1 milligram per patch. In further or additional embodiments, the amount of cellular material is from about 1 picogram to about 1 microgram per patch. In further or additional embodiments, the amount of cellular material is from about 1 picogram to about 1 nanogram per patch. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms per patch. In further or additional embodiments, the amount of cellular material, including nucleic acids such as RNA or DNA, or a polypeptide such as a protein, comprises an amount that is from about 50 microgram to 1 milligram, 50 microgram to 50 milligrams, 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms per patch. In further or additional embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram per patch. In further or additional embodiments, the amount of cellular material is from about 1 picogram to about 50 microgram per patch. In further or additional embodiments, the cellular material comprises an amount that is from about 1 picogram to about 15 micrograms, about 1 picogram to about 10 micrograms, about 1 picogram to about 50 micrograms, 50 picogram to about 1 microgram, about 1 picogram to about 50 micrograms, about 1 picogram to about 100 micrograms, about 1 picogram to about 200 micrograms, about 1 picogram to about 500 micrograms, about 1 picogram to about 750 micrograms, 500 picogram to 1 microgram, from about 100 picograms to about 500 microgram, from about 500 picograms to about 100 microgram, from about 750 picograms to about 1 microgram, from about 1 nanogram to about 750 microgram, from about 100 nanogram to about 500 microgram, or from about 1 nanogram to about 500 microgram per patch. In further or additional embodiments, the amount of cellular material is from about 1 picogram to about 1 gram per patch. In further or additional embodiments, the cellular material comprises an amount that is from about 50 microgram to about 1 milligram, from about 500 microgram to 1 milligram, from about 100 picograms to about 500 milligram, from about 500 picograms to about 100 milligram, from about 750 picograms to about 1 milligram, from about 1 nanogram to about 750 milligram, or from about 1 nanogram to about 500 milligram per patch.

### Computer Program

The methods, software, media, and systems disclosed herein comprise at least one computer processor, or use of the same. In some instances, the computer processor comprises a computer program. In some instances, a computer program includes a sequence of instructions, executable in the digital processing device's CPU, written to perform a specified task. In some instances, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program, in some embodiments, are written in various versions of various languages.

The functionality of the computer readable instructions, in certain embodiments, are combined or distributed as desired in various environments. In some instances, a computer program comprises one sequence of instructions. In some instances, a computer program comprises a plurality of sequences of instructions. In some instances, a computer program is provided from one location. In some instances, a computer program is provided from a plurality of locations. In some instances, a computer program includes one or more software modules. In some instances, a computer program includes, in part or in whole, one or more web applications, one or more mobile applications, one or more standalone applications, one or more web browser plug-ins, extensions, add-ins, or add-ons, or combinations thereof.

### Web Application

In some instances, a computer program includes a web application. In light of the disclosure provided herein, those of skill in the art will recognize that a web application, in certain embodiments, utilizes one or more software frameworks and one or more database systems. In some instances, a web application is created upon a software framework such as Microsoft® .NET or Ruby on Rails (RoR). In some instances, a web application utilizes one or more database systems including, by way of non-limiting examples, relational, non-relational, feature oriented, associative, and XML database systems. Suitable relational database systems includes, by way of non-limiting examples, Microsoft® SQL Server, mySQL™, and Oracle®. Those of skill in the art will also recognize that a web application, in certain embodiments, is written in one or more versions of one or more languages. In some instances, a web application is written in one or more markup languages, presentation definition languages, client-side scripting languages, server-side coding languages, database query languages, or combinations thereof. In some instances, a web application is written to some extent in a markup language such as Hypertext Markup Language (HTML), Extensible Hypertext Markup Language (XHTML), or eXtensible Markup Language (XML). In some instances, a web application is written to some extent in a presentation definition language such as Cascading Style Sheets (CSS). In some instances, a web application is written to some extent in a client-side scripting language such as Asynchronous Javascript and XML (AJAX), Flash® Actionscript, Javascript, or Silverlight®. In some instances, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some instances, a web application is written to some extent in a database query language such as Structured Query Language (SQL). In some instances, a web application integrates enterprise server products such as IBM® Lotus Domino®. In some instances, a web application includes a media player element. In some instances, a media player element utilizes one or more of many suitable multimedia technologies including, by way of non-limiting examples, Adobe® Flash®, HTML 5, Apple®QuickTime®, Microsoft® Silverlight®, Java™, and Unity®.

### Mobile Application

In some instances, a computer program includes a mobile application provided to a mobile digital processing device. In some instances, the mobile application is provided to a mobile digital processing device at the time it is manufactured. In some instances, the mobile application is provided to a mobile digital processing device via the computer network described herein.

In some instances, the mobile application is created by techniques known to those of skill in the art using hardware, languages, and development environments known to the art. Those of skill in the art will recognize that mobile applications, in certain embodiments, are written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments, in some instances, are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. In some instances, other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some instances, a computer program includes a standalone application, which is a program that is run as an independent computer process, not an add-on to an existing process, e.g., not a plug-in. Those of skill in the art will recognize that standalone applications are often compiled. In some instances, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB .NET, or combinations thereof. Compilation are often performed, at least in part, to create an executable program. In some instances, a computer program includes one or more executable complied applications.

### Web Browser Plug-In

In some instances, a computer program includes a web browser plug-in. In computing, a plug-in, in some instances, is one or more software components that add specific functionality to a larger software application. In some instances, makers of software applications support plug-ins to enable third-party developers to create abilities which extend an application, to support easily adding new features, and to reduce the size of an application. In some instances, when supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some instances, the toolbar comprises one or more web browser extensions, add-ins, or add-ons. In some instances, the toolbar comprises one or more explorer bars, tool bands, or desk bands.

In view of the disclosure provided herein, those of skill in the art will recognize that several plug-in frameworks, in some instances, are available that enable development of plug-ins in various programming languages, including, by way of non-limiting examples, C++, Delphi, Java™, PHP, Python™, and VB NET, or combinations thereof.

In some instances, web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some instances, web browser is a mobile web browser. In some instances, the mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules, in certain embodiments, are created by techniques known to those of skill in the art using machines, software, and languages known to the art. The software modules disclosed herein, in certain embodiments, are implemented in a multitude of ways. In some instances, a software module comprises a file, a section of code, a programming feature, a programming structure, or combinations thereof. In some instances, a software module comprises a plurality of files, a plurality of sections of code, a plurality of programming features, a plurality of programming structures, or combinations thereof. In some instances, the one or more software modules comprises, by way of non-limiting examples, a web application, a mobile application, and a standalone application. In some instances, software modules are in one computer program or application. In some instances, software modules are in more than one computer program or application. In some instances, software modules are hosted on one machine. In some instances, software modules are hosted on more than one machine. In some instances, software modules are hosted on cloud computing platforms. In some instances, software modules are hosted on one or more machines in one location. In some instances, software modules are hosted on one or more machines in more than one location.

### Databases

The medium, method, and system disclosed herein comprise one or more databases, or use of the same. In view of the disclosure provided herein, those of skill in the art will recognize that many databases, in certain embodiments, are suitable for storage and retrieval of geologic profile, operator activities, division of interest, and/or contact information of royalty owners. Suitable databases include, by way of non-limiting examples, relational databases, non-relational databases, feature oriented databases, feature databases, entity-relationship model databases, associative databases, and XML databases. In some instances, a database is internet-based. In some instances, a database is web-based. In some instances, a database is cloud computing-based. In some instances, a database is based on one or more local computer storage devices.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### Embodiments

Provided herein are numbered embodiments 1-47. Embodiment 1. A system for preparing a sample for non-invasive differential gene expression analysis comprising: providing a skin sample from a subject suspected of having one or more skin conditions, wherein the sample was obtained using a non-invasive sampling method; enriching RNAs from the skin sample; obtaining data comprising measurements from the RNAs; determining expression levels for one or more genes from the measurements; and applying a gene classifier to identify the skin sample is indicative of the one more skin conditions. Embodiment 2. A system for preparing a sample for non-invasive differential gene expression analysis, comprising: a communication interface that receives data comprising measurements of RNAs over a communication network, the measurements having been obtained from a skin sample using a non-invasive sampling method, and the skin sample having been obtained from a subject suspected of having one or more skin conditions; and a computer in communication with the communication interface, wherein the computer comprises one or more computer processors and computer readable medium comprising machine-executable code that, upon execution by the one or more computer processors, implements a method comprising: applying a classifier to the measurements to identify the skin sample indicative of the one or more skin conditions. Embodiment 3. A method for preparing a sample for non-invasive differential gene expression analysis, comprising: receiving or obtaining data comprising measurements of RNAs obtained from a skin sample adhered to an adhesive patch, the skin sample having been obtained from a subject suspected of having a one or more skin diseases; and applying a classifier to the measurements to identify the skin sample indicative of the one or more skin disease. Embodiment 4. The system or method of any one of embodiments 1-3, wherein the one or more skin conditions comprises SCC (squamous cell carcinoma) or AK (actinic keratosis). Embodiment 5. The system or method of any one of embodiments 1-3, wherein the one or more skin conditions comprises squamous cell carcinoma in situ (isSCC) or invasive squamous cell carcinoma (ivSCC). Embodiment 6. The system of any one of embodiments 1-4, wherein the non-invasive sampling method comprises adhering one or more adhesive patches. Embodiment 7. The method of embodiment 3, comprising obtaining the skin sample by adhering the adhesive patch to a skin area of the subject. Embodiment 8. The system or method of any one of embodiments 1-7, comprising obtaining the measurements by performing RNA-sequencing (RNA-seq). Embodiment 9. The system or method of any one of embodiments 1-8, wherein the RNAs comprise an mRNA. Embodiment 10. The system or method of embodiment 9, wherein the mRNA encodes a protein that plays a role in keratinization, developmental biology, metabolism of vitamins or cofactors, neutrophil degranulation, NOD-like receptor signaling, or an innate immune system. Embodiment 11. The system or method of embodiment 9 or 10, wherein the mRNA comprises transcripts of 1, 2, 3, 4, 5, 6, 7, 8, 9, 10, 11, 12, 13, 14, or 15 of: AD000090.1, CASP14, CXCL8, FABP5, IVNS1ABP, KRT1, KRT2, KRT5, KRT6C, NAMPT, NCOA1, RAB5B, SCAF11, SLC2A3, STK4, or TPM4. Embodiment 12. The system or method of any one of embodiments 9-11, wherein the mRNA comprises a transcript of AD000090.1, CASP14, CXCL8, FABP5, IVNS1ABP, KRT1, KRT2, KRT5, KRT6C, NAMPT, or SLLC2A3. Embodiment 13. The system or method of any one of embodiments 9-11, wherein the mRNA comprises a transcript of CAPZA2, IVNS1ABP, NCOA1, RAB5B, SCAF11, STK4, or TPM4. Embodiment 14. The system or method of any one of embodiments 9-13, wherein the mRNA is indicative of SCC. Embodiment 15. The system or method of any one of embodiments 1-14, wherein the SCC comprises squamous cell carcinoma in situ (isSCC) or invasive squamous cell carcinoma (ivSCC). Embodiment 16. The system or method of any one of embodiments 1-14, wherein the method further comprises recommending an SCC treatment or an AK treatment based on the identification. Embodiment 17. A system for preparing a sample for non-invasive differential gene expression analysis, comprising: Embodiment a communication interface that receives data comprising measurements of RNAs over a communication network, the measurements having been obtained from a skin sample adhered to an adhesive patch, and the skin sample having been obtained from a subject suspected of having SCC comprises squamous cell carcinoma in situ (isSCC) or invasive squamous cell carcinoma (ivSCC); and a computer in communication with the communication interface, wherein the computer comprises one or more computer processors and computer readable medium comprising machine-executable code that, upon execution by the one or more computer processors, implements a method comprising: applying a classifier to the measurements to identify the skin sample indicative of the isSCC or ivSCC. Embodiment 18. A method for preparing a sample for non-invasive differential gene expression analysis, comprising: receiving or obtaining data comprising measurements of RNAs obtained from a skin sample adhered to an adhesive patch, the skin sample having been obtained from a subject suspected of having SCC comprises squamous cell carcinoma in situ (isSCC) or invasive squamous cell carcinoma (ivSCC); and applying a classifier to the measurements to identify the skin sample indicative of the isSCC or ivSCC. Embodiment 19. The method of embodiment 18, comprising obtaining the skin sample by adhering the adhesive patch to a skin area of the subject. Embodiment 20. The method of embodiment 18 or 19, comprising obtaining the measurements by performing RNA-sequencing (RNA-seq). Embodiment 21. The system or method of any one of embodiments 17-20, wherein the RNAs comprise mRNA. Embodiment 22. The system or method of embodiment 21, wherein the mRNA comprises transcripts of 1, 2, 3, 4, 5, 6, 7, 8, 9, 10, 11, 12, 13, 14, 15, 16, 17, 18, 19, 20, 21, 22, 23, 24, 25, 26, 27, or 28 of: AC105460.1, ARC, CSAR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP4-6, KRTAP9-4, MAGED1, MT-CO2, PIM1, RN7SL752P, SERPINA1, SLC2A3, SPRR2A, SRGN, or U3. Embodiment 23. The system or method of embodiment 21 or 22, wherein the mRNA comprises a transcript of AC105460.1, ARC, COL6A1, EDN1, FAM110C, FOSL1, IL36G, JUN, PIM1, or RN7SL752P. Embodiment 24. The system or method of embodiment 21 or 23, wherein the mRNA comprises a transcript of CYFIP1, DDX3X, ITGA3, MAGED1, MT-CO2, or SPRR2A. Embodiment 25. The system or method of any one of embodiments 21-24, wherein the mRNA is indicative of isSCC. Embodiment 26. The system or method of embodiment 21 or 22, wherein the mRNA comprises a transcript of C5AR1, G0S2, KRTAP1-3, KRTAP1-5, KRTAP3-1, KRTAP4-6, KRTAP9-4, SERPINA1, SLC2A3, or SRGN. Embodiment 27. The system or method of embodiment 21 or 22, wherein the mRNA comprises a transcript of EFCAB2, KRTAP4-6, KRTAP9-4, or U3. Embodiment 28. The system or method of any one of embodiments 21-27, wherein the mRNA is indicative of ivSCC. Embodiment 29. The system or method of any one of embodiments 17-28, wherein the method further comprises recommending an isSCC treatment or an ivSCC treatment based on the identification. Embodiment 30. The system or method of any one of embodiments 1-29 wherein at least one mRNA transcript in the sample comprising SCC is upregulated compared to a control sample comprising AK. Embodiment 31. The system or method of embodiment 30, wherein the mRNA comprises a transcript of one or more of IVNS1ABP, RAB5B, CAPZA2, STK4, SCAF11, NCOA1, and CBL. Embodiment 32. The system or method of any one of embodiments 1-31 wherein at least one mRNA transcript in the sample comprising SCC is downregulated compared to a control sample comprising AK. Embodiment 33. The system or method of any one of embodiments 1-31 wherein at least one mRNA transcript in the sample comprising ivSCC is upregulated compared to a control sample comprising isSCC. Embodiment 34. The system or method of embodiment 33, wherein the mRNA comprises EFCAB2, KRTAP4-6, KRTAP9-4, and U3. Embodiment 35. The system or method of any one of embodiments 1-34 wherein at least one mRNA transcript in the sample comprising ivSCC is downregulated compared to a control sample comprising isSCC. Embodiment 36. The system or method of embodiment 35, wherein the mRNA comprises a transcript of one or more of DDX3X, CYFIP1, ITGA3, MAGED1, SPRR2Am, MT-CO2. Embodiment 37. The system or method of any one of embodiments 1-36, wherein the measurements comprise sequencing reads. Embodiment 38. The system or method of embodiment 37, wherein the sequencing reads are obtained by RNA-seq. Embodiment 39. The system or method of embodiment 37 or 38, wherein the method further comprises removing genomic DNA reads from the sequencing reads. Embodiment 40. The system or method of embodiment 39, wherein removing genomic DNA reads from the sequencing reads comprises identifying or quantifying exonic reads and non-exonic reads. Embodiment 41. The system or method of embodiment 39 or 40, wherein removing genomic DNA reads from the sequencing reads comprises excluding non-exonic reads from exonic. Embodiment 42. The system or method of any one of embodiments 37-41, wherein the sequencing reads are obtained using a next generation sequencing method. Embodiment 43. The system or method of embodiment 42 wherein next generation sequencing comprises single-molecule real-time (SMRT) sequencing, Polony sequencing, sequencing by synthesis, sequencing by ligation, reversible terminator sequencing, proton detection sequencing, ion semiconductor sequencing, nanopore sequencing, electronic sequencing, pyrosequencing, Maxam-Gilbert sequencing, chain termination sequencing, +S sequencing, and sequencing by synthesis. Embodiment 44. The system or method of any one of embodiments 1-43, wherein the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis. Embodiment 45. The system or method of any one of embodiments 1-44, wherein the skin sample comprises cells from the stratum corneum. Embodiment 46. The system or method of any one of embodiments 1-45, wherein the subject is a mammal. Embodiment 47. The system or method of any one of embodiments 1-45, wherein the subject is a human.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1

### Non-Invasive Gene Expression Assay for Differential Gene Expression

Non-invasive adhesive patches were used to obtain 89 samples from subjects for transcription analysis of select genes. Various skin areas of subjects was sampled, and sample information is shown in Table 2.

TABLE 2

| | Sub DX | | | | | | | | | | | |
|---|---|---|---|---|---|---|---|---|---|---|---|---|
| Working | (isSCC/iv | | | | history of | Fam history | NMSC in | | | | | |
| Number | SCC/NA) | Gender | Age | Location | melanoma | of melanoma | past year | sun | smoking | Fitzpatrick | sunscreen | Patient |
| 8 | isSCC | F | 83 | Foot | Y | N | 3 | Y | NA | I | Y | 37 |
| 10 | isSCC | M | 89 | Temple | N | N | 4 | Y | no | I | Y | 14 |
| 50 | isSCC | F | 64 | Nasal | N | N | 1 | Y | no | I | Y | 12 |
| 53 | isSCC | F | 74 | Hand | N | N | 1 | Y | no | I | Y | 48 |
| 54 | isSCC | F | 85 | Scalp | Y | NA | 3 | N | no | I | N | 10 |
| 61 | isSCC | F | 69 | Temple | N | N | 8 | Y | no | I | Y | |
| 62 | isSCC | M | 81 | Temple | N | N | 5 | Y | no | I | Y | 52 |
| 64 | isSCC | M | 81 | Temple | N | N | 5 | Y | no | I | Y | 52 |
| 68 | isSCC | M | 86 | Cheek | N | N | 6 | Y | no | I | Y | 57 |
| 78 | isSCC | F | 73 | Leg | N | N | 1 | Y | yes | I | Y | 59 |
| 84 | isSCC | F | 87 | Temple | N | N | NA | Y | no | I | Y | 4 |
| 85 | isSCC | M | 93 | Neck | N | N | 2 | N | former | I | N | 1 |
| 86 | isSCC | M | 91 | Shin | N | N | 1 | Y | former | I | Y | 3 |
| 1 | ivSCC | F | 62 | Shin | NA | NA | 1 | Y | former | III | Y | 41 |
| 2 | ivSCC | M | 78 | Hand | Y | N | 15 | Y | former | II | Y | 39 |
| 5 | ivSCC | M | 78 | Calf | N | N | 1 | N | former | II | N | 36 |
| 9 | ivSCC | M | 94 | Ear | N | N | 3 | N | no | II | N | 13 |
| 11 | ivSCC | M | 76 | Calf | N | N | 2 | Y | no | I | Y | 15 |
| 14 | ivSCC | M | 86 | Cheek | N | NA | 8 | NA | former | II | NA | 7 |
| 16 | ivSCC | M | 75 | Forehead | N | NA | 1 | Y | former | I | Y | 30 |
| 17 | ivSCC | F | 94 | Neck | N | N | 4 | N | former | II | N | 29 |
| 18 | ivSCC | M | 74 | Scalp | N | N | 1 | N | former | I | N | 31 |
| 21 | ivSCC | F | 68 | Leg | Y | Y | NA | Y | no | III | Y | 28 |
| 22 | ivSCC | M | 44 | Lip | N | N | 1 | Y | no | II | Y | 32 |
| 23 | ivSCC | M | 60 | Forehead | N | Y | 4 | Y | no | I | Y | 33 |
| 24 | ivSCC | M | 87 | Lip | N | N | 1 | N | no | II | N | 35 |
| 33 | ivSCC | M | 88 | Chest | N | N | 2 | N | no | II | N | 40 |
| 39 | ivSCC | M | 87 | Forearm | N | N | 1 | Y | no | I | Y | 21 |
| 43 | ivSCC | M | 90 | Forearm | N | N | 5 | Y | former | I | Y | 26 |
| 45 | ivSCC | F | 94 | Neck | N | N | 3 | N | former | I | N | 18 |
| 63 | ivSCC | M | 69 | Scalp | N | NA | 6 | N | NA | II | N | 51 |
| 65 | ivSCC | M | 79 | Ear | N | NA | 1 | Y | no | I | Y | 50 |
| 67 | ivSCC | F | 79 | Calf | N | N | 2 | Y | NA | I | Y | 56 |
| 75 | ivSCC | M | 77 | Chest | N | N | 4 | Y | no | II | Y | 61 |
| 87 | ivSCC | M | 93 | Scalp | N | N | 2 | N | former | I | N | 1 |
| 13 | AK | M | 73 | Cheek | NA | NA | NA | NA | NA | II | NA | 6 |
| 15 | AK | NA | NA | Leg | NA | NA | NA | NA | NA | NA | NA | 8 |
| 20 | AK | M | 73 | Hand | Y | N | 1 | Y | no | II | Y | 27 |
| 37 | AK | F | 94 | Ear | N | N | 3 | N | former | I | N | 18 |
| 40 | AK | M | 82 | Jaw | NA | NA | NA | NA | NA | II | NA | 23 |
| 48 | AK | F | 72 | Cheek | NA | NA | NA | NA | NA | II | NA | 11 |
| 49 | AK | M | 72 | Cheek | NA | NA | NA | NA | NA | II | NA | 9 |
| 51 | AK | M | 92 | Forearm | N | N | 1 | Y | no | I | Y | 16 |
| 55 | AK | F | 85 | Scalp | Y | NA | 3 | N | no | I | N | 10 |
| 57 | AK | M | 60 | Foot | NA | NA | NA | NA | NA | NA | NA | 53 |
| 81 | AK | M | 58 | Shin | NA | NA | NA | NA | NA | I | NA | 2 |
| 88 | AK | M | 91 | Shin | N | N | 1 | Y | former | I | Y | 3 |

Legend: Y = yes; N = no; M = male; F = female; NA: not applicable.

In some instances, multiple samples were obtained from the same patient, according to Table 3.

TABLE 3

Samples from the same patient

| | | | |
|---|---|---|---|
| AK | 88 | isSCC | 86 |
| AK | 55 | isSCC | 54 |
| AK | 37 | ivSCC | 45 |
| isSCC | 85 | ivSCC | 87 |

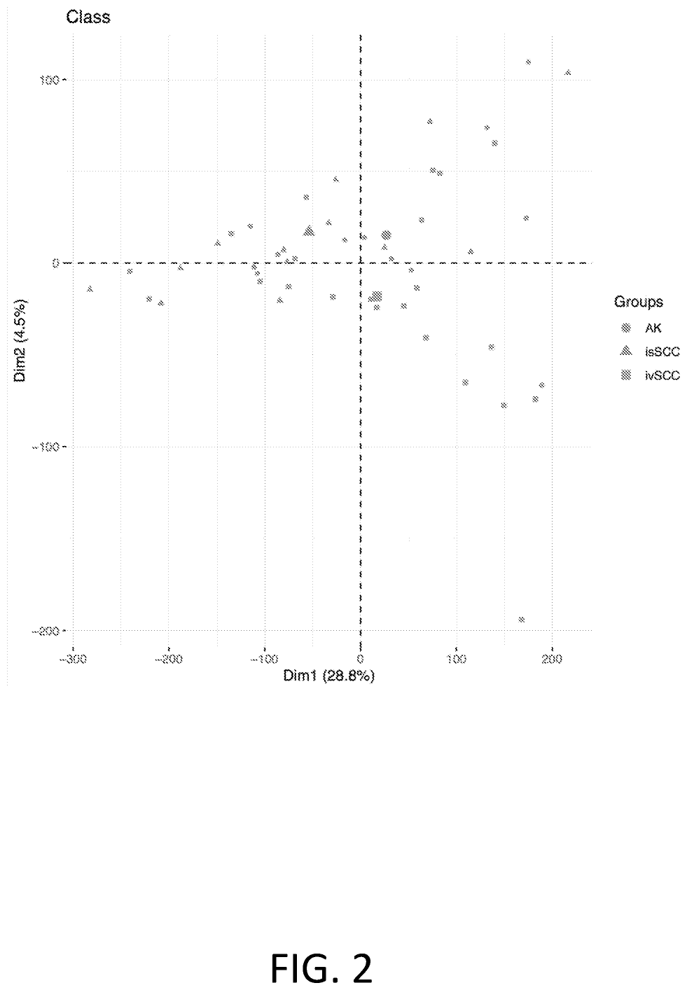

RNA was isolated from the patches, reverse transcribed to generate a cDNA library, amplified, and sequenced using next generation sequencing. Sequencing metrics are shown in FIGS. 1A-1C, including input reads (millions), uniquely mapped reads (millions), and mapping statistics (percentage). Contamination of gDNA (genomic DNA) was observed both by intergenic reads and using a genome browser (FIGS. 1D-1E). A further analysis was performed on 47 samples (12 AK, 13 isSCC, and 22 ivSCC, FIG. 2).

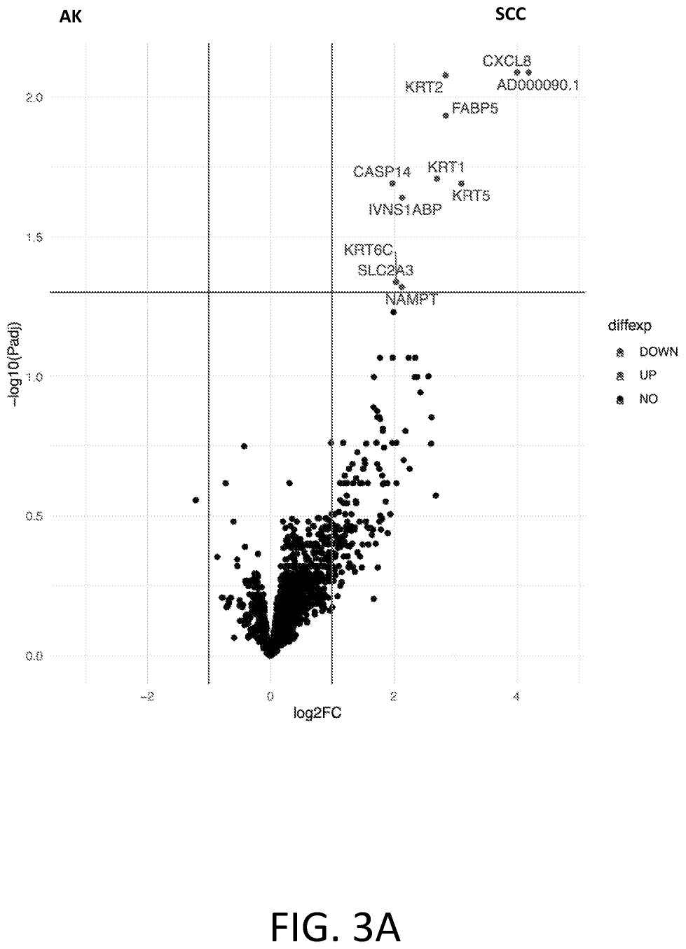

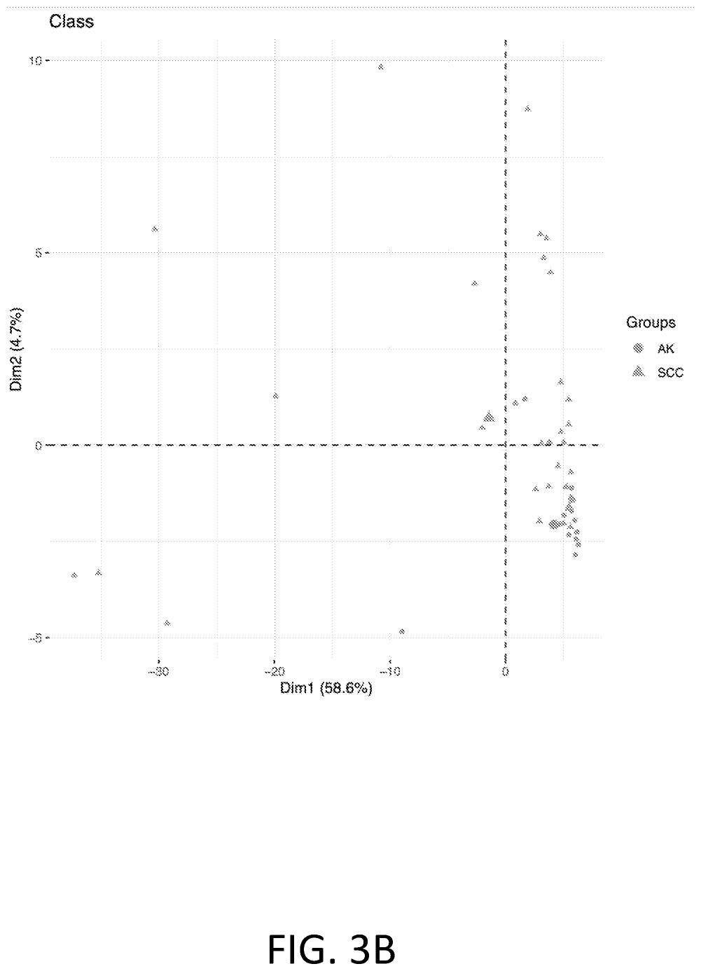

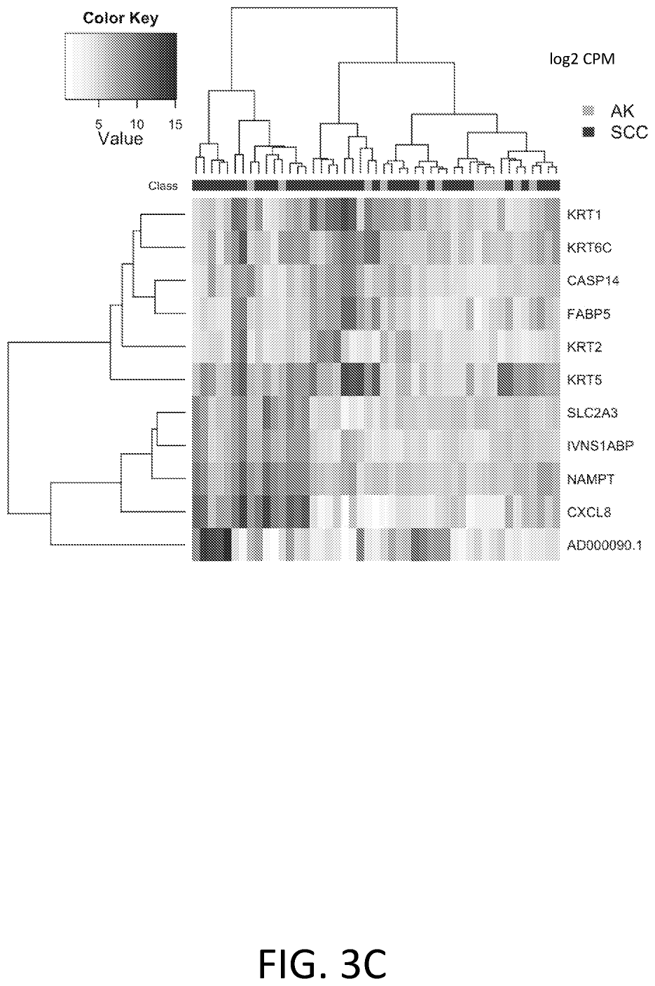

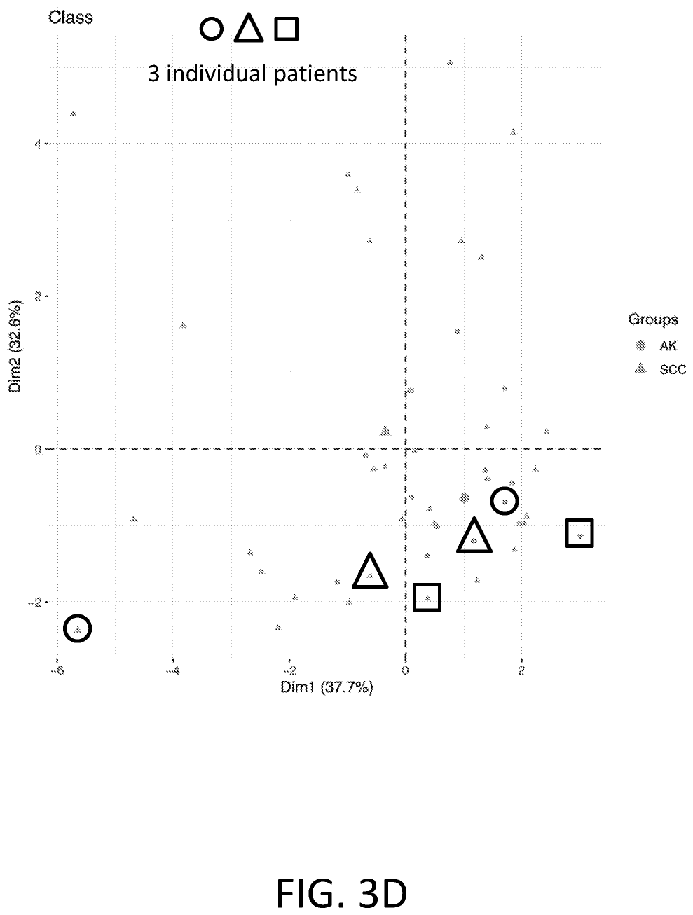

A differentially expressed gene (DEG) list having FC>2 and adj p<0.05 for AK vs. SCC was identified by generating a 2D plot of expression values against AK and SCC sample types. Genes KRT1, KRT6C, CASP14, FABP5, KRT2, KRT5, SLC2A3, IVNS1ABP, NAMPT, CXCL8, and AD000090.1 were found to be differentially expressed at a higher rate in SCC vs. AK samples (FIGS. 3A-3C). Samples from the same patient did not group together (FIG. 3D). Expression data is summarized in Table 4A. Pathways involving the 11 DEGs were identified and quantified in Table 4B.

TABLE 4A

| Gene symbol | log2FoldChange | Adjusted p value |
|---|---|---|
| CXCL8 | 4.00 | 8.16E−03 |
| AD000090.1 | 4.19 | 8.16E−03 |
| KRT2 | 2.84 | 8.34E−03 |
| FABP5 | 2.85 | 1.17E−02 |
| KRT1 | 2.70 | 1.96E−02 |
| KRT5 | 3.10 | 2.04E−02 |
| CASP14 | 1.98 | 2.04E−02 |
| IVNS1ABP | 2.14 | 2.29E−02 |
| SLC2A3 | 2.04 | 4.59E−02 |
| KRT6C | 2.04 | 4.59E−02 |
| NAMPT | 2.13 | 4.79E−02 |

TABLE 4B

| q-value | pathway |
|---|---|
| 9.23E−08 | Keratinization |
| 1.69E−04 | Developmental Biology |
| 7.82E−03 | Metabolism of water-soluble vitamins and cofactors |
| 8.39E−03 | Neutrophil degranulation |
| 1.02E−02 | Metabolism of vitamins and cofactors |
| 1.02E−02 | NOD-like receptor signaling pathway |
| 4.14E−02 | Innate Immune System |

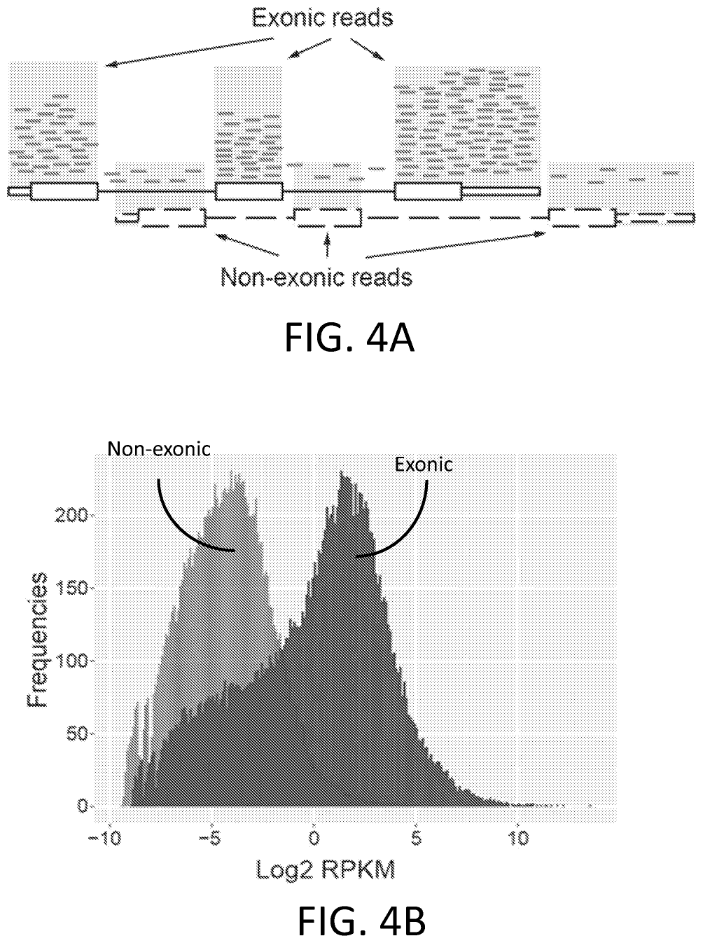

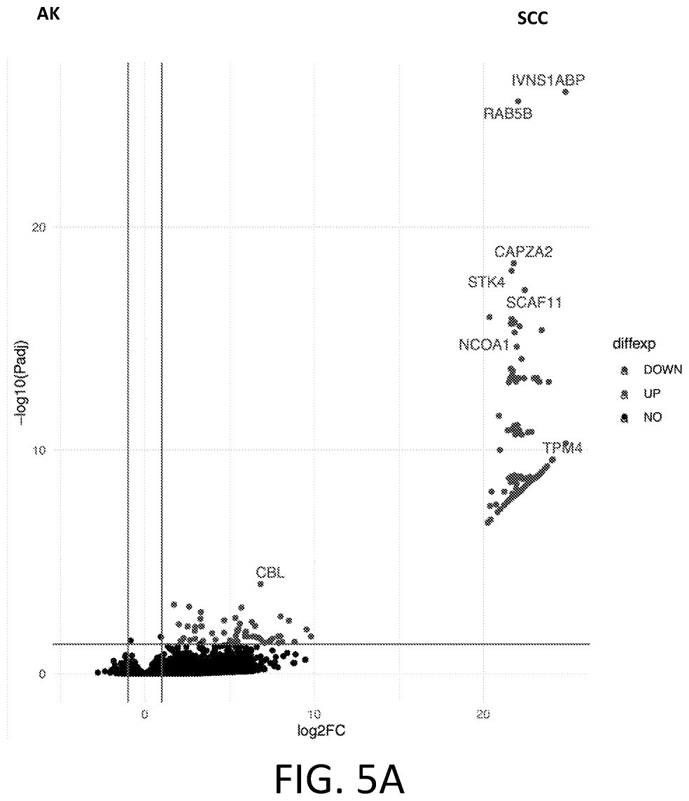

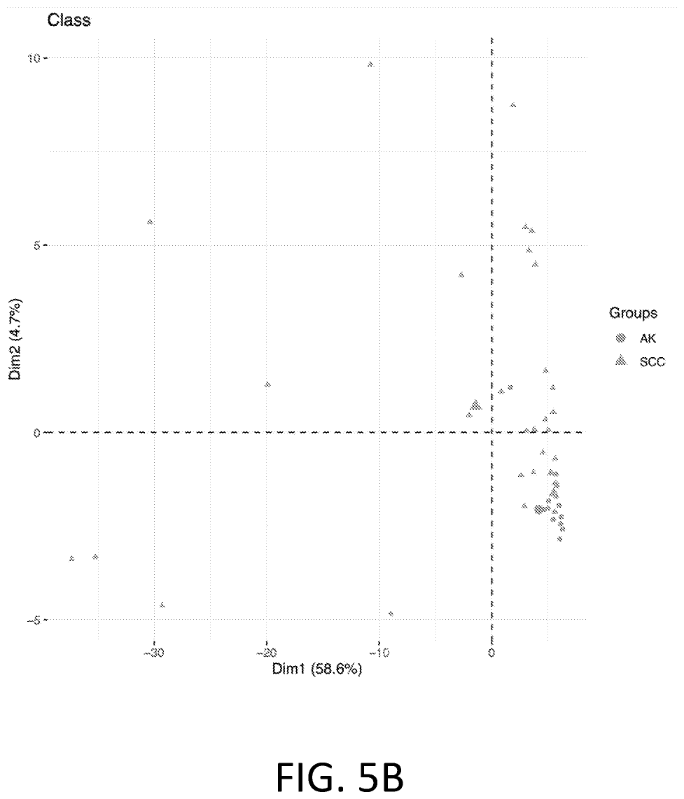

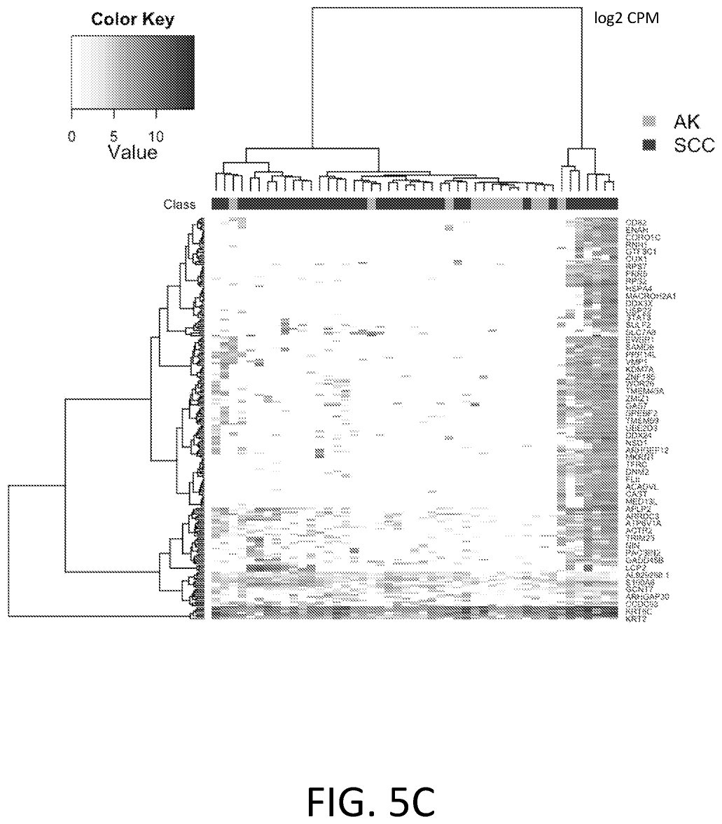

To increase sensitivity and reduce the influence of gDNA reads in the data set, both exonic and non-exonic reads were quantified (FIG. 4A). Next, non-exonic reads were excluded from exonic reads. (FIG. 4B). After reducing gDNA reads, 219 DEGS (FC>2 and adj p<0.05) were identified as shown in FIGS. 5A-5C. Genes identified as differentially upregulated in SCC vs. AK included IVNS1ABP, RAB5B, CAPZA2, STK4, SCAF11, NCOA1, CBL and others. Expression data is summarized in Table 5A. Pathways related to these DEGS are shown in Table 5B.

TABLE 5A

| Gene symbol | log2FoldChange | Adjusted p value | Gene symbol | log2Fold Change | Adjusted p value |
|---|---|---|---|---|---|
| IVNS1ABP | 24.86 | 8.63E−27 | DNAJB1 | 22.14 | 7.47E−09 |
| RAB5B | 22.05 | 2.33E−26 | NIN | 22.10 | 7.85E−09 |
| CAPZA2 | 21.80 | 4.28E−19 | HSPA9 | 22.11 | 7.85E−09 |
| STK4 | 21.67 | 9.35E−19 | ANXA11 | 22.07 | 8.26E−09 |
| SCAF11 | 22.44 | 6.87E−18 | RPL18 | 22.07 | 8.26E−09 |
| SHOC2 | 20.36 | 1.11E−16 | XIST | 22.03 | 8.91E−09 |
| SLC20A2 | 21.67 | 1.34E−16 | ANXA8L1 | 22.00 | 9.24E−09 |
| OGT | 21.85 | 1.90E−16 | ZMIZ1 | 21.99 | 9.24E−09 |
| GAB1 | 21.61 | 2.20E−16 | GJB6 | 21.72 | 9.24E−09 |
| DDX24 | 21.73 | 2.23E−16 | METAP2 | 21.96 | 9.63E−09 |
| HBP1 | 22.15 | 2.87E−16 | PRR5 | 21.97 | 9.63E−09 |
| GADD45B | 23.45 | 4.26E−16 | HSPA4 | 21.97 | 9.63E−09 |
| LCP2 | 21.85 | 5.48E−16 | FLII | 21.94 | 9.86E−09 |
| NCOA1 | 21.97 | 2.40E−15 | PSMD8 | 21.94 | 9.86E−09 |
| SLC2A3 | 22.26 | 8.40E−15 | FCGBP | 21.93 | 9.86E−09 |
| GJB3 | 21.61 | 2.28E−14 | HADHA | 21.94 | 9.86E−09 |
| PRR14L | 21.74 | 2.95E−14 | CD109 | 21.93 | 9.91E−09 |
| BTAF1 | 21.69 | 4.65E−14 | EEF1D | 21.92 | 9.91E−09 |
| NBPF20 | 22.05 | 6.13E−14 | RAN | 21.90 | 1.03E−08 |
| GAS5 | 22.11 | 6.13E−14 | KRTAP4-6 | 21.90 | 1.03E−08 |
| CD82 | 21.75 | 6.13E−14 | ACACA | 21.88 | 1.05E−08 |
| VMP1 | 22.42 | 6.13E−14 | NFAT5 | 21.85 | 1.10E−08 |
| NCKAP1 | 23.18 | 6.13E−14 | ENAH | 21.84 | 1.13E−08 |
| CUL3 | 21.95 | 6.13E−14 | RPL37A | 21.83 | 1.15E−08 |
| CLCN3 | 22.99 | 6.13E−14 | TFRC | 21.82 | 1.15E−08 |
| RAB2A | 21.76 | 6.13E−14 | COPB2 | 21.80 | 1.20E−08 |
| RLIM | 21.59 | 6.13E−14 | ATP1B3 | 21.79 | 1.20E−08 |
| ZNF185 | 23.11 | 6.57E−14 | ZNF292 | 21.79 | 1.20E−08 |
| WDR26 | 23.33 | 8.78E−14 | MKNK2 | 21.78 | 1.22E−08 |
| RPL31 | 23.25 | 8.78E−14 | RNH1 | 21.76 | 1.25E−08 |
| ACAP2 | 21.93 | 8.78E−14 | DICER1 | 21.76 | 1.25E−08 |
| TMEM45A | 23.87 | 9.02E−14 | BMPR2 | 21.75 | 1.25E−08 |
| THEMIS2 | 21.52 | 9.33E−14 | CDC42 | 21.72 | 1.32E−08 |
| KPNB1 | 20.92 | 2.98E−12 | PYGL | 21.71 | 1.32E−08 |
| ARHGEF12 | 22.03 | 7.63E−12 | COX4I1 | 21.71 | 1.32E−08 |
| ITGAV | 21.84 | 8.16E−12 | MORF4L2 | 21.71 | 1.32E−08 |
| DNM2 | 21.68 | 1.19E−11 | TBC1D14 | 21.71 | 1.33E−08 |
| RPS15 | 21.64 | 1.24E−11 | CUX1 | 21.69 | 1.36E−08 |
| SREBF2 | 22.12 | 1.33E−11 | SLC44A2 | 21.67 | 1.41E−08 |
| FAM20C | 21.44 | 1.33E−11 | CD9 | 21.66 | 1.41E−08 |
| DBI | 21.96 | 1.46E−11 | MED13L | 21.66 | 1.41E−08 |
| USP34 | 22.84 | 1.57E−11 | TBC1D23 | 21.65 | 1.42E−08 |
| UBXN7 | 21.87 | 1.59E−11 | HOOK3 | 21.56 | 1.68E−08 |
| DDX6 | 22.65 | 1.63E−11 | EIF4H | 21.55 | 1.71E−08 |
| TMEM59 | 22.20 | 1.94E−11 | GTF3C1 | 21.50 | 1.87E−08 |
| TMPRSS11D | 22.28 | 2.08E−11 | DOCK9 | 21.40 | 2.26E−08 |
| OXR1 | 21.88 | 2.08E−11 | ITGB1 | 20.76 | 2.84E−08 |
| TPM4 | 24.87 | 5.16E−11 | EXPH5 | 21.25 | 2.97E−08 |
| FOXO3 | 20.97 | 1.03E−10 | PDCD6IP | 21.24 | 3.03E−08 |
| CAST | 24.11 | 2.67E−10 | CORO1C | 21.23 | 3.04E−08 |
| DDX3X | 24.05 | 2.96E−10 | GATAD2A | 20.40 | 3.27E−08 |
| LMNA | 23.76 | 5.39E−10 | PAFAH1B1 | 21.01 | 4.62E−08 |
| RPS2 | 23.64 | 6.88E−10 | NOSIP | 20.84 | 6.40E−08 |
| STAT3 | 23.47 | 9.73E−10 | NSD1 | 20.43 | 1.36E−07 |
| CAP1 | 23.45 | 9.88E−10 | RPL13 | 20.26 | 1.85E−07 |
| PALLD | 23.37 | 1.16E−09 | CBL | 6.84 | 0.00010178 |
| CSNK1A1 | 23.30 | 1.32E−09 | AC068643.2 | 1.72 | 0.00085124 |
| VDAC2 | 21.84 | 1.39E−09 | GRAMD4P2 | 2.61 | 0.00104351 |
| NPEPPS | 21.88 | 1.42E−09 | AC092821.1 | 5.71 | 0.00114697 |
| TOR1AIP2 | 22.22 | 1.57E−09 | KRT2 | 3.32 | 0.0018348 |
| HMOX2 | 21.67 | 1.57E−09 | ARF1 | 8.03 | 0.00284205 |
| KAT6A | 23.18 | 1.57E−09 | PTPRS | 5.35 | 0.00332072 |
| WAC | 22.12 | 1.59E−09 | KRT16 | 3.28 | 0.00366903 |
| SF3B1 | 23.15 | 1.61E−09 | SERPINB12 | 4.69 | 0.00427381 |
| STEAP4 | 22.75 | 1.64E−09 | NIBAN1 | 8.51 | 0.00438893 |
| ZNF638 | 21.63 | 1.71E−09 | TFG | 6.31 | 0.00505826 |
| PPIA | 23.09 | 1.73E−09 | APLP2 | 5.62 | 0.00602784 |
| EWSR1 | 22.17 | 1.77E−09 | AL929288.1 | 2.01 | 0.00644413 |
| SAMD9 | 23.07 | 1.77E−09 | FABP5P7 | 6.51 | 0.00765583 |
| RNF10 | 21.85 | 1.80E−09 | STK17B | 3.33 | 0.00777858 |
| CTSB | 23.05 | 1.80E−09 | AC097376.3 | 3.35 | 0.00784858 |
| MKRN1 | 21.91 | 1.86E−09 | AC093484.2 | 2.53 | 0.00794472 |
| TTC39B | 22.00 | 1.86E−09 | FABP5 | 2.97 | 0.00843869 |
| ATP6V1G1 | 21.51 | 1.86E−09 | TMED9 | 5.54 | 0.01049752 |
| PTBP3 | 22.52 | 1.89E−09 | IGF2R | 9.55 | 0.01109362 |
| USP22 | 21.79 | 1.95E−09 | BRMS1 | 5.46 | 0.01179185 |
| OPTN | 21.89 | 1.95E−09 | KRT1 | 2.96 | 0.0124939 |
| SULF2 | 22.96 | 1.97E−09 | FAM83B | 5.97 | 0.01345013 |
| EPPK1 | 22.94 | 2.01E−09 | RCOR1 | 5.46 | 0.01384438 |
| CXADR | 22.34 | 2.02E−09 | ARRDC3 | 4.68 | 0.01514014 |
| HNRNPH1 | 21.96 | 2.02E−09 | SARM1 | 5.93 | 0.01615398 |
| GAS7 | 22.72 | 2.03E−09 | CPD | 3.98 | 0.01615398 |
| RPL13A | 22.91 | 2.03E−09 | CCDC93 | 5.38 | 0.02103337 |
| KDM7A | 21.67 | 2.03E−09 | OSM | 8.01 | 0.02148435 |
| RPS7 | 22.89 | 2.10E−09 | PAIP2 | 5.92 | 0.02177459 |
| ATP2B4 | 22.86 | 2.21E−09 | ACTR2 | 7.88 | 0.02201331 |
| GNAI3 | 22.73 | 2.23E−09 | SLC2A1 | 6.08 | 0.02270753 |
| NFE2L1 | 22.83 | 2.29E−09 | LIMA1 | 6.40 | 0.02270753 |
| TCP1 | 21.81 | 2.37E−09 | ACSL1 | 9.82 | 0.02275549 |
| BTF3 | 22.77 | 2.53E−09 | ATP6V1A | 6.57 | 0.02379222 |
| HNRNPA2B1 | 22.75 | 2.58E−09 | TRIM25 | 6.72 | 0.02595586 |
| HNRNPU | 22.75 | 2.58E−09 | KRT10 | 2.28 | 0.02671183 |
| SLC3A2 | 21.63 | 2.79E−09 | NCOA4 | 7.54 | 0.02810836 |
| UBE2D3 | 22.70 | 2.81E−09 | DSG1 | 2.97 | 0.02860565 |
| CCT5 | 22.61 | 3.31E−09 | NOLC1 | 4.18 | 0.0318233 |
| CHD9 | 21.96 | 3.32E−09 | ZFP36L1 | 6.85 | 0.03188651 |
| ACADVL | 22.48 | 4.19E−09 | GCNT7 | 2.67 | 0.03241412 |
| RPL28 | 21.94 | 5.47E−09 | LDHA | 7.45 | 0.03320667 |
| OAZ1 | 22.33 | 5.64E−09 | SDCCAG8 | 5.03 | 0.03385209 |
| STAT1 | 22.28 | 6.17E−09 | S100A6 | 3.43 | 0.03590456 |
| GPX3 | 22.27 | 6.17E−09 | VKORC1 | 2.10 | 0.03590456 |
| GRN | 22.24 | 6.52E−09 | SCLT1 | 5.45 | 0.03606407 |
| OS9 | 22.21 | 6.93E−09 | KRT6C | 2.16 | 0.03676659 |
| SPINT2 | 22.20 | 6.98E−09 | PROM2 | 7.35 | 0.03676659 |
| CAPRIN1 | 22.18 | 7.17E−09 | ARHGAP30 | 5.47 | 0.03778786 |
| KDM2A | 22.14 | 7.46E−09 | KLHL28 | 5.21 | 0.03878742 |
| USF2 | 20.48 | 7.46E−09 | SLC7A8 | 8.86 | 0.03905709 |
| EPHA4 | 21.24 | 7.46E−09 | THBD | 8.12 | 0.03978778 |
| MACROH2A1 | 22.14 | 7.46E−09 | PACSIN2 | 7.06 | 0.04360537 |
| | | | KANK1 | 7.89 | 0.04360537 |

TABLE 5B

| q-value | pathway |
|---|---|
| 2.71E−04 | Eukaryotic Translation Elongation |
| 2.71E−04 | Cellular responses to stress |
| 2.71E−04 | Cellular response to starvation |
| 2.71E−04 | Cellular responses to external stimuli |
| 2.71E−04 | L13a-mediated translational silencing of Ceruloplasmin |
| | expression |
| 2.71E−04 | GTP hydrolysis and joining of the 60S ribosomal subunit |
| 2.71E−04 | Peptide chain elongation |
| 2.71E−04 | Eukaryotic Translation Termination |
| 2.71E−04 | Selenocysteine synthesis |
| 2.71E−04 | Cap-dependent Translation Initiation |
| 2.71E−04 | Eukaryotic Translation Initiation |
| 2.84E−04 | Nonsense Mediated Decay (NMD) independent of the Exon |
| | Junction Complex (EJC) |
| 4.13E−04 | Response of EIF2AK4 (GCN2) to amino acid deficiency |
| 4.43E−04 | Formation of a pool of free 40S subunits |
| 4.83E−04 | Basigin interactions |
| 4.83E−04 | Nonsense Mediated Decay (NMD) enhanced by the Exon |
| | Junction Complex (EJC) |
| 4.83E−04 | Nonsense-Mediated Decay (NMD) |
| 6.81E−04 | SRP-dependent cotranslational protein targeting to membrane |
| 8.85E−04 | Selenoamino acid metabolism |
| 1.08E−03 | Neutrophil degranulation |
| 2.95E−03 | Developmental Biology |
| 2.95E−03 | Interleukin-6 family signaling |
| 2.95E−03 | Ferroptosis - Homo sapiens (human) |
| 2.95E−03 | Immune System |
| 2.95E−03 | Cytoprotection by HMOX1 |
| 2.95E−03 | Coronavirus disease - COVID-19 - Homo sapiens (human) |
| 2.95E−03 | Chromatin modifying enzymes |
| 2.95E−03 | Chromatin organization |
| 2.95E−03 | Ribosome - Homo sapiens (human) |
| 3.05E−03 | Diseases of signal transduction by growth factor receptors and |
| | second messengers |
| 3.61E−03 | Disease |
| 3.78E−03 | Cellular response to chemical stress |
| 4.27E−03 | Interleukin-6 signaling |
| 5.11E−03 | Endocytosis - Homo sapiens (human) |
| 5.43E−03 | Innate Immune System |
| 5.52E−03 | Bacterial invasion of epithelial cells - Homo sapiens (human) |
| 5.88E−03 | Beta oxidation of palmitoyl-CoA to myristoyl-CoA |
| 8.51E−03 | Golgi Associated Vesicle Biogenesis |
| 1.17E−02 | Hemostasis |
| 1.28E−02 | Membrane Trafficking |
| 1.48E−02 | Signaling by FGFR1 in disease |
| 1.48E−02 | Deregulated CDK5 triggers multiple neurodegenerative |
| | pathways in Alzheimer's disease models |
| 1.48E−02 | Neurodegenerative Diseases |
| 1.48E−02 | Signaling by cytosolic FGFR1 fusion mutants |
| 1.57E−02 | RHO GTPase cycle |
| 1.86E−02 | Translation |
| 1.86E−02 | RND1 GTPase cycle |
| 1.86E−02 | Signaling by PDGFR in disease |
| 2.01E−02 | trans-Golgi Network Vesicle Budding |
| 2.01E−02 | Vesicle-mediated transport |
| 2.01E−02 | Clathrin-mediated endocytosis |
| 2.28E−02 | Diseases of programmed cell death |
| 2.34E−02 | Signaling by MET |
| 2.48E−02 | Signaling by FGFR in disease |
| 2.60E−02 | Metabolism of RNA |
| 2.85E−02 | Potential therapeutics for SARS |
| 3.27E−02 | Metabolism of amino acids and derivatives |
| 3.36E−02 | Vitamin C (ascorbate) metabolism |
| 3.45E−02 | Thyroid hormone signaling pathway - Homo sapiens (human) |
| 4.14E−02 | Interleukin-9 signaling |
| 4.41E−02 | Iron uptake and transport |
| 4.48E−02 | Keratinization |
| 4.52E−02 | Cell surface interactions at the vascular wall |
| 4.52E−02 | Signaling by Receptor Tyrosine Kinases |
| 4.52E−02 | Metabolism of proteins |
| 4.55E−02 | Interleukin-27 signaling |
| 4.55E−02 | Folding of actin by CCT/TriC |
| 4.55E−02 | Regulation of cholesterol biosynthesis by SREBP (SREBF) |
| 4.55E−02 | FGFR1 mutant receptor activation |
| 4.55E−02 | Transferrin endocytosis and recycling |

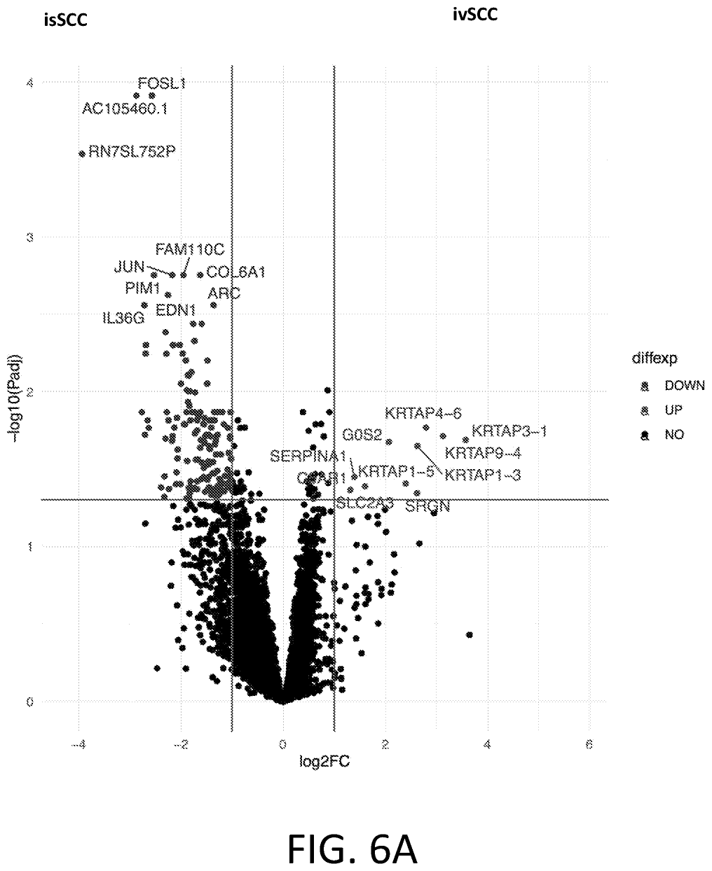

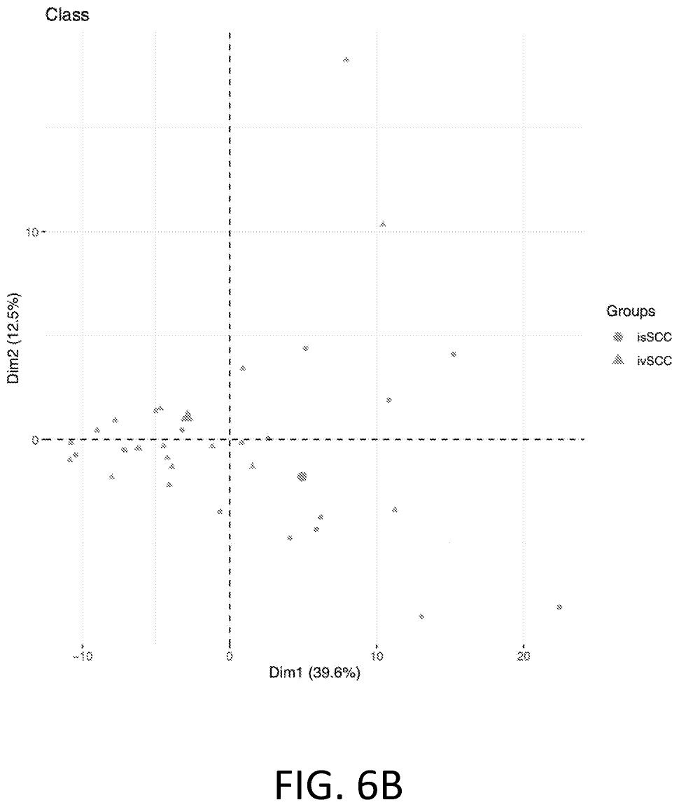

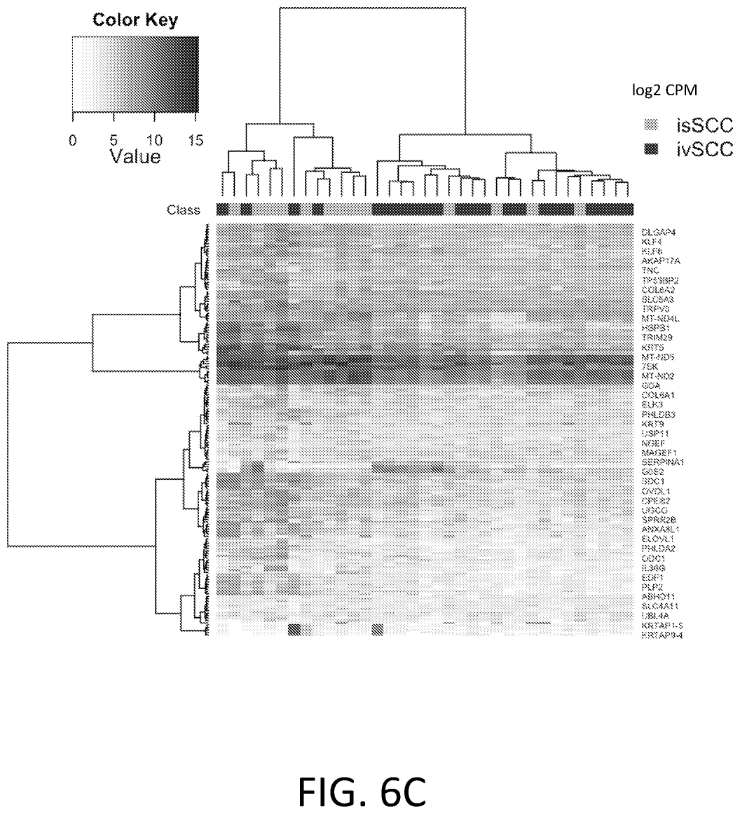

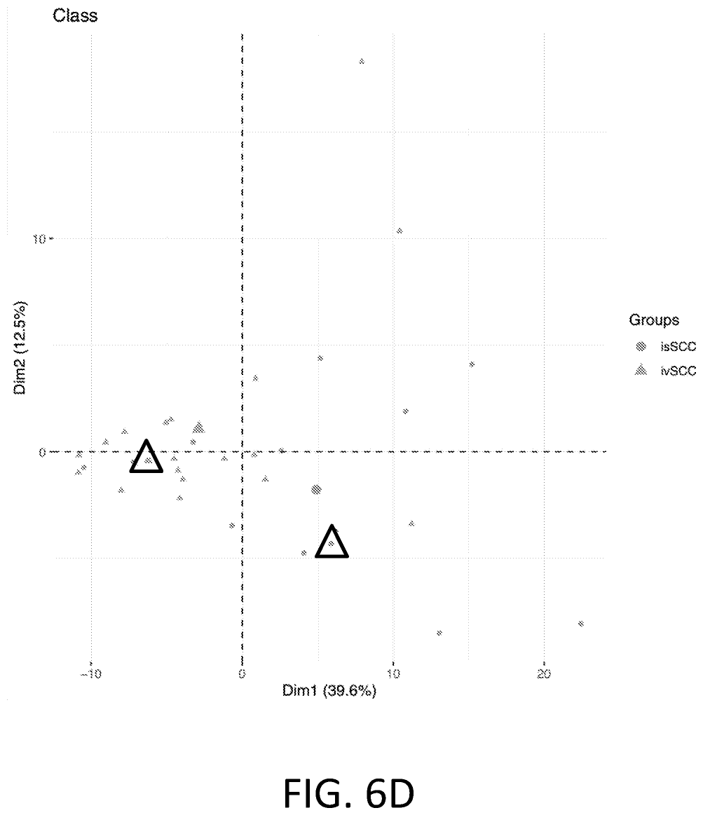

DEGs were next identified (FC>2 and adj p<0.05) for isSCC vs. ivSCC samples, as shown in FIGS. 6A-6C. Genes which were differentially upregulated in ivSCC relative to isSCC were SERPINA1, G0S2, KRTAP4-6, KRTAP3-1, KRTAP9-4, KRTAP1-3, SRGN, SLC2A3, KRTAP1-5, and C5AR1. Genes which were differentially downregulated in isSCC relative to ivSCC included FOSL1, AC105460.1, RN7SL752P, FAM110C, JUN, PIM1, JL36G, EDN1, ARC, CO_6A1, and others. Samples from the same patient did not group together regarding ivSCC and isSCC (FIG. 6D). Expression data is summarized in Table 6A. Pathways related to these DEGS are shown in Table 6B.

TABLE 6A

| Gene symbol | log2FoldChange | Adjusted p value | Gene symbol | log2FoldChange | Adjusted p value |
|---|---|---|---|---|---|
| AC105460.1 | −2.87 | 1.22E−04 | ELK3 | −1.55 | 2.10E−02 |
| FOSL1 | −2.57 | 1.22E−04 | G0S2 | 2.07 | 2.12E−02 |
| RN7SL752P | −3.94 | 2.91E−04 | SLC4A11 | −1.54 | 2.15E−02 |
| JUN | −2.17 | 1.77E−03 | AHNAK | −1.33 | 2.20E−02 |
| FAM110C | −1.95 | 1.77E−03 | KRTAP1-3 | 2.63 | 2.25E−02 |
| PIM1 | −2.53 | 1.77E−03 | SPIN4 | −1.45 | 2.25E−02 |
| COL6A1 | −1.63 | 1.77E−03 | TM4SF1 | −1.62 | 2.31E−02 |
| EDN1 | −2.25 | 2.38E−03 | HSPH1 | −1.43 | 2.35E−02 |
| IL36G | −2.72 | 2.77E−03 | SERPINB7 | −2.06 | 2.37E−02 |
| ARC | −1.36 | 2.77E−03 | GDA | −1.06 | 2.49E−02 |
| TYRP1 | −1.76 | 3.65E−03 | PADI1 | −1.22 | 2.55E−02 |
| DLGAP4 | −1.59 | 3.65E−03 | RAP2B | −1.31 | 2.55E−02 |
| MT-CO3 | −2.30 | 4.13E−03 | SPRR2B | −2.07 | 2.70E−02 |
| ANXA8L1 | −1.73 | 4.71E−03 | DNAH1 | −1.02 | 2.72E−02 |
| ODC1 | −2.69 | 5.00E−03 | RBBP6 | −1.66 | 2.75E−02 |
| EREG | −2.15 | 5.00E−03 | MT-ATP8 | −1.71 | 2.76E−02 |
| MT-CO2 | −2.16 | 5.00E−03 | CXCL14 | −1.88 | 2.81E−02 |
| MT-ND4L | −2.02 | 5.00E−03 | MT-ND5 | −1.46 | 2.83E−02 |
| MTATP6P1 | −1.97 | 5.69E−03 | BNIPL | −1.37 | 2.85E−02 |
| KRT5 | −2.69 | 5.69E−03 | PLP2 | −1.59 | 2.88E−02 |
| ALOX12B | −2.29 | 5.70E−03 | SGK1 | −1.46 | 2.95E−02 |
| CPEB2 | −1.90 | 6.30E−03 | CCNL1 | −1.55 | 3.11E−02 |
| CEP72 | −1.48 | 6.30E−03 | AC083801.2 | −1.09 | 3.18E−02 |
| HIP1R | −1.79 | 7.48E−03 | USO1 | −1.29 | 3.25E−02 |
| FERMT1 | −1.87 | 7.83E−03 | UGCG | −2.09 | 3.33E−02 |
| LGALS7 | −1.85 | 7.89E−03 | EDF1 | −1.38 | 3.37E−02 |
| XDH | −1.48 | 8.89E−03 | SERPINB5 | −1.35 | 3.37E−02 |
| MT-CO1 | −2.00 | 8.89E−03 | PHLDA1 | −1.03 | 3.41E−02 |
| MT-ND1 | −1.88 | 9.81E−03 | SLC44A2 | −1.14 | 3.51E−02 |
| TNFRSF12A | −1.83 | 9.95E−03 | PHLDA2 | −2.32 | 3.53E−02 |
| MT-ND2 | −1.86 | 9.95E−03 | CSNK1A1 | −1.46 | 3.56E−02 |
| MT-CYB | −1.72 | 1.01E−02 | AQP3 | −1.56 | 3.56E−02 |
| MT-ND3 | −1.86 | 1.17E−02 | SERPINA1 | 1.39 | 3.56E−02 |
| MT-ND4 | −1.83 | 1.23E−02 | DENND2C | −1.52 | 3.62E−02 |
| NGEF | −1.03 | 1.36E−02 | TJP2 | −1.60 | 3.67E−02 |
| FBXO45 | −1.73 | 1.36E−02 | TNC | −1.03 | 3.67E−02 |
| CDKN1A | −2.28 | 1.36E−02 | NR1D1 | −1.25 | 3.67E−02 |
| TUT7 | −1.89 | 1.36E−02 | SUB1 | −1.48 | 3.70E−02 |
| HSPA8 | −1.62 | 1.36E−02 | ABHD11 | −1.02 | 3.70E−02 |
| TRPV3 | −1.26 | 1.36E−02 | CRCT1 | −1.81 | 3.80E−02 |
| EEF2 | −1.79 | 1.36E−02 | KLF6 | −1.69 | 3.80E−02 |
| TICAM1 | −1.19 | 1.36E−02 | MBD1 | −1.22 | 3.80E−02 |
| PI3 | −2.76 | 1.36E−02 | AC004816.1 | −1.06 | 3.86E−02 |
| COL6A2 | −1.29 | 1.36E−02 | KRTAP1-5 | 2.40 | 3.93E−02 |
| AKAP17A | −1.35 | 1.36E−02 | VEGFA | −1.14 | 3.96E−02 |
| MT-ATP6 | −1.66 | 1.44E−02 | MYOF | −1.22 | 3.96E−02 |
| HBEGF | −2.08 | 1.48E−02 | TRIM29 | −1.03 | 3.96E−02 |
| PPARD | −1.54 | 1.48E−02 | EIF3CL | −2.01 | 3.96E−02 |
| TENT5B | −1.34 | 1.53E−02 | ZNF185 | −2.10 | 3.96E−02 |
| ECM1 | −1.44 | 1.53E−02 | HPSE | −1.27 | 4.05E−02 |
| S100A9 | −2.65 | 1.53E−02 | PDAP1 | −1.06 | 4.06E−02 |
| S100A16 | −1.82 | 1.53E−02 | SLC2A3 | 1.61 | 4.10E−02 |
| MAGEF1 | −1.35 | 1.53E−02 | CYSRT1 | −1.82 | 4.14E−02 |
| HSPB1 | −2.24 | 1.53E−02 | GPR50 | −1.25 | 4.14E−02 |
| KLF4 | −1.69 | 1.53E−02 | UBL4A | −1.45 | 4.14E−02 |
| UBE2S | −1.40 | 1.53E−02 | PHLDB2 | −1.05 | 4.15E−02 |
| ALG13 | −1.37 | 1.53E−02 | 7SK | −2.39 | 4.15E−02 |
| ZBTB17 | −1.42 | 1.53E−02 | SLC5A3 | −1.07 | 4.15E−02 |
| FOXN2 | −1.56 | 1.62E−02 | ELOVL1 | −1.85 | 4.19E−02 |
| YWHAQ | −1.94 | 1.63E−02 | GBA2 | −1.02 | 4.19E−02 |
| ATG9B | −1.16 | 1.63E−02 | KIF5B | −1.31 | 4.19E−02 |
| RAB38 | −1.92 | 1.63E−02 | OVOL1 | −1.84 | 4.19E−02 |
| PRPF6 | −1.55 | 1.63E−02 | SDR9C7 | −1.73 | 4.19E−02 |
| MTCO1P12 | −1.40 | 1.71E−02 | UBC | −2.23 | 4.25E−02 |
| KRTAP4-6 | 2.80 | 1.71E−02 | ANXA2 | −1.34 | 4.25E−02 |
| JUP | −2.13 | 1.71E−02 | KRT9 | −1.23 | 4.25E−02 |
| NEAT1 | −2.63 | 1.72E−02 | DSC3 | −1.74 | 4.25E−02 |
| ZNF114 | −1.08 | 1.72E−02 | TP53BP2 | −1.47 | 4.28E−02 |
| FBXO11 | −1.68 | 1.78E−02 | SDCBP2 | −1.69 | 4.28E−02 |
| SDC1 | −1.73 | 1.87E−02 | BMP2K | −1.31 | 4.33E−02 |
| S100A8 | 2.70 | 1.90E−02 | ZBTB43 | −1.38 | 4.33E−02 |
| CCDC191 | −1.28 | 1.90E−02 | PHLDB3 | −1.34 | 4.33E−02 |
| RPL7 | −1.50 | 1.90E−02 | C5AR1 | 1.32 | 4.33E−02 |
| PPP2R5B | −1.45 | 1.90E−02 | YWHAE | −1.28 | 4.40E−02 |
| TBC1D2 | −1.52 | 1.93E−02 | TRIB3 | −1.15 | 4.40E−02 |
| SFN | −2.16 | 1.94E−02 | ITGB1 | −1.53 | 4.44E−02 |
| KRTAP9-4 | 3.13 | 1.94E−02 | RPL23A | −1.39 | 4.52E−02 |
| USP11 | −1.31 | 1.94E−02 | SRGN | 2.62 | 4.53E−02 |
| PTMS | −1.03 | 1.95E−02 | SIK1 | −1.83 | 4.53E−02 |
| XIST | −1.42 | 1.95E−02 | FOSB | −1.25 | 4.59E−02 |
| FLNB | −1.03 | 2.00E−02 | ALOXE3 | −1.40 | 4.64E−02 |
| RN7SKP71 | −2.30 | 2.01E−02 | MAP4K4 | −1.33 | 4.69E−02 |
| STK38L | −1.61 | 2.05E−02 | PLCXD1 | −1.27 | 4.69E−02 |
| KRTAP3-1 | 3.57 | 2.05E−02 | FLG | −2.32 | 4.80E−02 |
| RHCG | −2.17 | 2.08E−02 | SH3BGRL3 | −1.89 | 4.90E−02 |
| DUSP14 | −1.32 | 2.08E−02 | TOP1 | −1.83 | 4.90E−02 |

TABLE 6B

| q-value | pathway |
|---|---|
| 2.23E−06 | Oxidative phosphorylation - Homo sapiens (human) |
| 2.24E−06 | Respiratory electron transport, ATP synthesis by |
| | chemiosmotic coupling, and heat production by uncoupling |
| | proteins. |
| 1.63E−05 | Parkinson disease - Homo sapiens (human) |
| 1.85E−05 | Respiratory electron transport |
| 3.02E−05 | Prion disease - Homo sapiens (human) |
| 3.09E−05 | The citric acid (TCA) cycle and respiratory electron transport |
| 3.46E−05 | Diabetic cardiomyopathy - Homo sapiens (human) |
| 7.41E−05 | Huntington disease - Homo sapiens (human) |
| 1.10E−04 | Thermogenesis - Homo sapiens (human) |
| 4.42E−04 | Amyotrophic lateral sclerosis - Homo sapiens (human) |
| 4.42E−04 | Alzheimer disease - Homo sapiens (human) |
| 4.42E−04 | Pathways of neurodegeneration - multiple diseases - Homo |
| | sapiens (human) |
| 2.88E−03 | TP53 Regulates Metabolic Genes |
| 5.56E−03 | Activation of BH3-only proteins |
| 6.01E−03 | Complex I biogenesis |
| 6.05E−03 | Chk1/Chk2(Cds1) mediated inactivation of Cyclin B:Cdk1 |
| | complex |
| 6.05E−03 | Keratinization |
| 6.10E−03 | Cellular responses to stress |
| 6.10E−03 | Uptake and function of diphtheria toxin |
| 7.32E−03 | Cellular responses to external stimuli |
| 7.32E−03 | Activation of BAD and translocation to mitochondria |
| 9.99E−03 | Signaling by Receptor Tyrosine Kinases |
| 1.05E−02 | NGF-stimulated transcription |
| 1.05E−02 | PI3K-Akt signaling pathway - Homo sapiens (human) |
| 1.21E−02 | Transcriptional Regulation by TP53 |
| 1.24E−02 | Formation of the cornified envelope |
| 1.25E−02 | Neutrophil degranulation |
| 1.33E−02 | Syndecan interactions |
| 1.96E−02 | Metal sequestration by antimicrobial proteins |
| 1.98E−02 | Apoptosis |
| 2.08E−02 | Intrinsic Pathway for Apoptosis |
| 2.08E−02 | ECM-receptor interaction - Homo sapiens (human) |
| 2.40E−02 | Synthesis of 12-eicosatetraenoic acid derivatives |
| 2.54E−02 | Developmental Biology |
| 2.54E−02 | IL-17 signaling pathway - Homo sapiens (human) |
| 2.62E−02 | Vesicle-mediated transport |
| 2.62E−02 | Programmed Cell Death |
| 2.62E−02 | Inhibition of Signaling by Overexpressed EGFR |
| 2.62E−02 | Signaling by Overexpressed Wild-Type EGFR in Cancer |
| 2.62E−02 | AUF1 (hnRNP D0) binds and destabilizes mRNA |
| 2.66E−02 | Downregulation of ERBB2 signaling |
| 2.66E−02 | Attenuation phase |
| 2.74E−02 | Nuclear Events (kinase and transcription factor activation) |
| 2.78E−02 | EGFR downregulation |
| 2.78E−02 | FOXO-mediated transcription |
| 2.79E−02 | Retrograde endocannabinoid signaling - Homo sapiens |
| | (human) |
| 2.79E−02 | EGFR interacts with phospholipase C-gamma |
| 2.79E−02 | CHL1 interactions |
| 2.82E−02 | Focal adhesion - Homo sapiens (human) |
| 2.97E−02 | Cytoprotection by HMOX1 |
| 3.02E−02 | Proteoglycans in cancer - Homo sapiens (human) |
| 3.02E−02 | Membrane Trafficking |
| 3.14E−02 | PI3K events in ERBB4 signaling |
| 3.28E−02 | Translocation of SLC2A4 (GLUT4) to the plasma membrane |
| 3.45E−02 | Toll Like Receptor 4 (TLR4) Cascade |
| 3.51E−02 | Human papillomavirus infection - Homo sapiens (human) |
| 3.78E−02 | PIP3 activates AKT signaling |
| 3.88E−02 | HSF1-dependent transactivation |
| 4.04E−02 | Regulation of localization of FOXO transcription factors |
| 4.04E−02 | Degradation of beta-catenin by the destruction complex |
| 4.28E−02 | MAPK6/MAPK4 signaling |
| 4.49E−02 | GRB2 events in EGFR signaling |
| 4.49E−02 | ERBB2 Activates PTK6 Signaling |
| 4.55E−02 | Cell cycle - Homo sapiens (human) |
| 4.62E−02 | Bladder cancer - Homo sapiens (human) |
| 4.69E−02 | Immune System |
| 4.69E−02 | Non-integrin membrane-ECM interactions |
| 4.69E−02 | Regulation of HSF1-mediated heat shock response |
| 4.69E−02 | SHC1 events in ERBB4 signaling |
| 4.69E−02 | SHC1 events in EGFR signaling |
| 4.98E−02 | Toll-like Receptor Cascades |

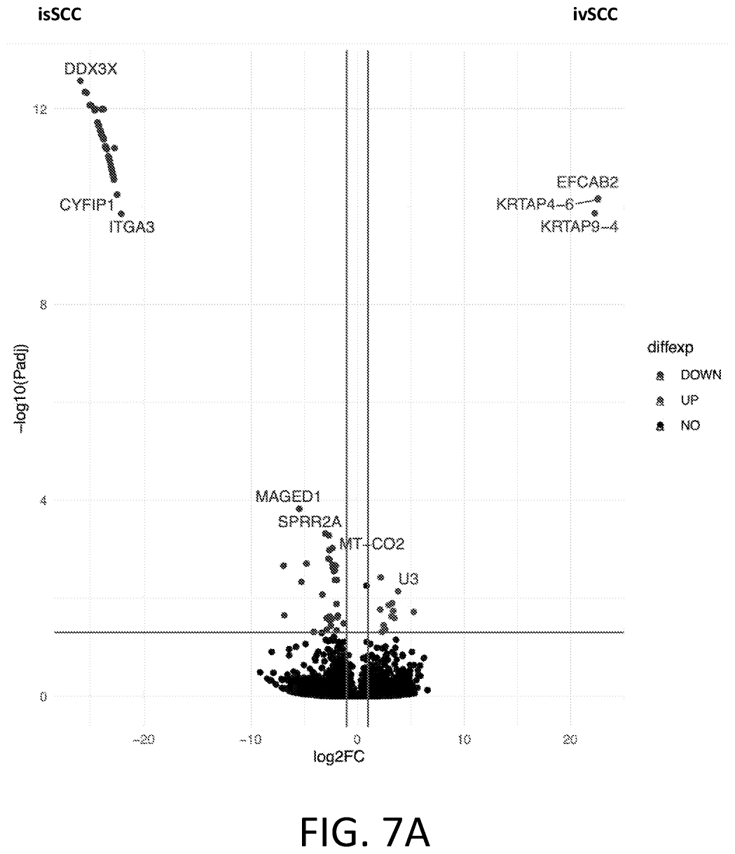

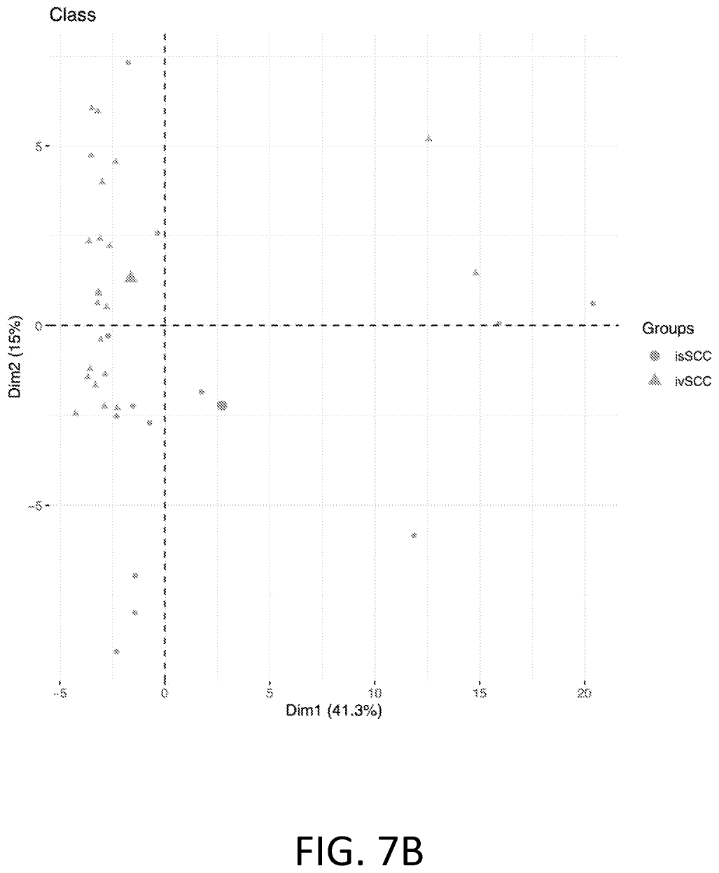

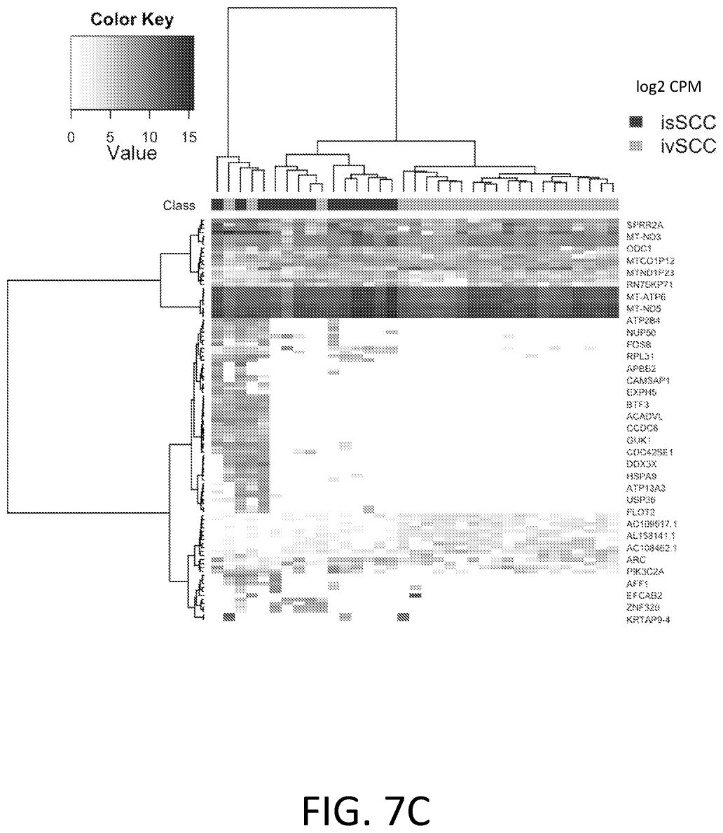

Genomic DNA correction was also performed to identify 101 DEGs (FC>2 and adj p<0.05) for isSCC vs. ivSCC samples, as shown in FIGS. 7A-7C. Genes which were differentially upregulated in ivSCC relative to isSCC included EFCAB2, KRTAP4-6, KRTAP9-4, U3, and others. Genes which were differentially downregulated in isSCC relative to ivSCC included DDX3X, CYFIP1, ITGA3, MAGED1, SPRR2Am, MT-CO2, and others. Expression data is summarized in Table 7A. Pathways related to these DEGS are shown in Table 7B.

TABLE 7A

| Gene symbol | log2FoldChange | Adjusted p value | Gene symbol | log2FoldChange | Adjusted p value |
|---|---|---|---|---|---|
| DDX3X | −25.94 | 2.68E−13 | HSPA9 | −22.85 | 2.42E−11 |
| EPPK1 | −25.52 | 4.51E−13 | APBB2 | −22.79 | 2.79E−11 |
| CAST | −25.36 | 4.70E−13 | CYFIP1 | −22.50 | 5.64E−11 |
| ZNF320 | −25.05 | 8.37E−13 | EFCAB2 | 22.57 | 6.75E−11 |
| LMNA | −24.97 | 8.40E−13 | KRTAP4-6 | 22.55 | 7.12E−11 |
| XIST | −24.90 | 8.59E−13 | KRTAP9-4 | 22.28 | 1.35E−10 |
| TACC2 | −23.76 | 1.01E−12 | ITGA3 | −22.11 | 1.39E−10 |
| GMPPA | −23.95 | 1.01E−12 | MAGED1 | −5.44 | 0.00014969 |
| NUP50 | −24.52 | 1.01E−12 | SPRR2A | −3.00 | 0.00048039 |
| AC006001.3 | −23.89 | 1.01E−12 | MT-CO2 | −2.66 | 0.00052628 |
| FOSB | −24.62 | 1.01E−12 | MTATP6P1 | −2.31 | 0.00094887 |
| PPIA | −24.57 | 1.07E−12 | MT-CO1 | −2.62 | 0.00105145 |
| ACADVL | −24.33 | 1.90E−12 | ODC1 | −2.70 | 0.00156627 |
| ATP2B4 | −24.22 | 2.19E−12 | MT-ND4L | −2.58 | 0.00162204 |
| MYO5A | −24.22 | 2.19E−12 | IL36G | −4.76 | 0.0019703 |
| ATP13A3 | −24.20 | 2.19E−12 | MT-ND1 | −2.31 | 0.00207342 |
| EXPH5 | −24.07 | 2.80E−12 | MTCO3P12 | −2.03 | 0.00216846 |
| NFAT5 | −24.07 | 2.80E−12 | TFAP2A | −6.91 | 0.00216846 |
| ATP2B1 | −24.00 | 3.19E−12 | MT-ATP6 | −2.23 | 0.00234875 |
| CD63 | −23.94 | 3.52E−12 | MT-ND4 | −2.29 | 0.00244551 |
| HSPH1 | −23.87 | 3.64E−12 | MT-CYB | −2.11 | 0.00274842 |
| PYGL | −23.90 | 3.64E−12 | MT-ND2 | −2.19 | 0.00280982 |
| UBE2D3 | −23.88 | 3.64E−12 | GOLGA8M | 2.19 | 0.00379483 |
| AC105460.1 | −23.86 | 3.64E−12 | MT-ND3 | −2.07 | 0.00424126 |
| CORO1C | −23.84 | 3.66E−12 | MTCO1P12 | −1.91 | 0.00426074 |
| OAZ1 | −23.82 | 3.78E−12 | ZBED4 | −5.23 | 0.00466712 |
| IFI16 | −23.77 | 4.18E−12 | U3 | 3.82 | 0.00724808 |
| VSIG10 | −23.63 | 5.81E−12 | KRT14 | −3.27 | 0.00846158 |
| CDC42SE1 | −22.73 | 6.31E−12 | AC138819.1 | 3.26 | 0.0127964 |
| GUK1 | −23.56 | 6.31E−12 | MT-ND5 | −1.94 | 0.01313289 |
| CIITA | −23.57 | 6.31E−12 | RPL7P29 | 2.95 | 0.01390439 |
| IFT172 | −23.58 | 6.31E−12 | AC019288.1 | 2.15 | 0.01707071 |
| TNRC6B | −23.54 | 6.31E−12 | AL158141.1 | 3.36 | 0.01820634 |
| BTF3 | −23.52 | 6.31E−12 | C1RL-AS1 | 5.29 | 0.01913536 |
| TMBIM6 | −23.48 | 6.56E−12 | MTND1P23 | −1.80 | 0.02253137 |
| MAFF | −23.49 | 6.56E−12 | RPL31 | −6.84 | 0.02253137 |
| CAMSAP1 | −23.49 | 6.56E−12 | ANKRD60 | 3.20 | 0.02347253 |
| OSBPL8 | −23.47 | 6.56E−12 | TACSTD2 | −2.60 | 0.02365633 |
| PHF20L1 | −23.45 | 6.65E−12 | ZNF165 | −1.86 | 0.02454536 |
| AFF1 | −23.31 | 9.26E−12 | AC109517.1 | 3.48 | 0.02548959 |
| PALLD | −23.31 | 9.26E−12 | RN7SKP71 | −2.87 | 0.02564563 |
| AP1B1 | −23.27 | 9.95E−12 | RANBP3 | −2.38 | 0.02636702 |
| FLOT2 | −23.26 | 1.00E−11 | SPRR2B | −2.56 | 0.02904274 |
| USP36 | −23.19 | 1.14E−11 | ZNF543 | −1.28 | 0.0324559 |
| ARHGEF1 | −23.19 | 1.14E−11 | U6 | 2.46 | 0.03581676 |
| RNH1 | −23.11 | 1.39E−11 | AL596188.1 | −2.46 | 0.03680118 |
| RAN | −23.05 | 1.58E−11 | AC108462.1 | 2.59 | 0.04332591 |
| CCDC6 | −23.01 | 1.70E−11 | ARC | −2.86 | 0.04431164 |
| CD9 | −22.95 | 1.94E−11 | EEF2 | −1.92 | 0.04531944 |
| RPL15 | −22.90 | 2.18E−11 | G0S2 | 2.32 | 0.04886986 |
| | | | PIK3C2A | −4.07 | 0.04907537 |

TABLE 7B

| q-value | pathway |
|---|---|
| 1.01E−07 | Oxidative phosphorylation - Homo sapiens (human) |
| 2.33E−07 | Respiratory electron transport, ATP synthesis by |
| | chemiosmotic coupling, and heat production by uncoupling |
| | proteins. |
| 4.79E−07 | Respiratory electron transport |
| 1.51E−06 | Diabetic cardiomyopathy - Homo sapiens (human) |
| 2.20E−06 | The citric acid (TCA) cycle and respiratory electron transport |
| 3.54E−06 | Thermogenesis - Homo sapiens (human) |
| 5.85E−06 | Parkinson disease - Homo sapiens (human) |
| 1.20E−05 | Prion disease - Homo sapiens (human) |
| 1.85E−05 | Amyotrophic lateral sclerosis - Homo sapiens (human) |
| 2.70E−05 | Huntington disease - Homo sapiens (human) |
| 1.07E−04 | Complex I biogenesis |
| 1.19E−04 | Alzheimer disease - Homo sapiens (human) |
| 8.23E−04 | Uptake and function of diphtheria toxin |
| 8.23E−04 | Retrograde endocannabinoid signaling - Homo sapiens |
| | (human) |
| 8.23E−04 | Pathways of neurodegeneration - multiple diseases - Homo |
| | sapiens (human) |
| 3.37E−03 | Regulation of ornithine decarboxylase (ODC) |
| 4.88E−03 | Metabolism |
| 7.65E−03 | Host Interactions of HIV factors |
| 8.42E−03 | Reduction of cytosolic Ca++ levels |
| 9.46E−03 | Cellular responses to stress |
| 1.09E−02 | Cellular responses to external stimuli |
| 1.25E−02 | Metabolism of polyamines |
| 2.21E−02 | Deregulated CDK5 triggers multiple neurodegenerative |
| | pathways in Alzheimer's disease models |
| 2.21E−02 | Neurodegenerative Diseases |
| 3.16E−02 | Diseases of programmed cell death |
| 3.32E−02 | Uptake and actions of bacterial toxins |
| 3.42E−02 | TP53 Regulates Metabolic Genes |
| 3.67E−02 | Platelet calcium homeostasis |
| 3.67E−02 | Basigin interactions |
| 3.67E−02 | Regulation of HSF1-mediated heat shock response |
| 4.32E−02 | Cardiac muscle contraction - Homo sapiens (human) |

### Example 2

Evaluation of Gene Classifiers for Nonmelanoma Skin Cancers Using qPCR Assays

Overview. A set of 37 genes was evaluated for its ability to classify nonmelanoma skin cancers (NMSCs), basal cell carcinoma (BCC) and squama cell carcinoma (SCC), from other lesions that they are commonly clinically confused with. The control set was split into two groups: actinic keratosis (AKs) and “other”, which refers to all other skin disease that might be confused with NMSCs.

Methods. Subjects. Samples were taken from DermTech internal clinical studies 14-03 and 17-06. Both studies enrolled subjects clinically suspected of BCC or SCC lesions as well as other skin diseases. Skin biopsies were collected from all enrolled subjects and were used for histopathological confirmation.

Skin Sampling. The DermTech adhesive skin collection kit (DermTech, La Jolla, CA) was used to collect skin samples from lesional skin and nearby non-lesional skin. Prior to application of the Smart Sticker™, the target skin was prepped with an alcohol pad to remove oils and then dry wicked with a gauze pad to remove any remaining moisture. Each kit contains a total of 4 Smart Stickers™ for sample collection. Smart StickerS™ were applied individually to the designated area and 5 circular motions with the thumb were used to ensure adhesion to the targeted skin. Using a pen, small marks were made on the skin after placement of the first tape-strip to ensure consistent placement of each subsequent tape-strip. Smart Stickers™ were removed slowly using standard precautions to prevent folding and eliminate potential sources of contamination. Once removed, the Smart Sticker™ was then placed onto the tri-fold sample collector. This process was repeated with the 2^{nd}, 3^{rd} and 4^{th} Smart Sticker™ on the same lesional/non-lesional skin. Once all 4 Smart Stickers™ were in the tri-fold collector, the collector was carefully folded and placed in a re-sealable bag for overnight shipment (the Smart Sticker™ and tri-fold collector are stable for at least 10 days at room temperature). Upon arrival at DermTech, samples were stored at −70° C. or colder until RNA extraction.

RNA Extraction and Gene Expression Analysis. RNA was extracted from Smart Stickers™ using a closed-tube, bead-based method. Briefly, Smart Stickers™ were enzymatically digested to extract the genomic material from the adhesive and nucleic acid isolated using magnetic beads on a Kingfisher Flex instrument (Thermo Fisher Scientific). RNA expression results were obtained for 37 genes using the relative-quantification RT-qPCR method on the QuantStudio7 Pro instrument according to the manufacturer's instructions (Thermo Fisher Scientific). Relative-quantification RT-qPCR referred to the expression changes of target genes compared to ACTB. Gene expression results were obtained by normalizing the signal of the target gene to the signal of control gene ACTB. RT-qPCR method was used based on the manufacturer's protocol for TaqPath 1-step RT-qPCR Master Mix (Thermo Fisher Scientific). Duplex reactions were used. Each reaction had an ACTB control assay and a target assay. The 36 target genes used Thermo Fisher Scientific designed assays for detection (assay identifiers listed as three letter codes after each gene).

Results. To calculate which genes best classified BCC from AK/other and SCC from AK/other, p-values were calculated from F-test ANOVA. A summary of individual genes ability to classify BCC (Table 8) and SCC (Table 9) from AK/other skin disorders was prepared. The p-values were calculated from F-test ANOVA. The genes were sorted by p-value.

TABLE 8

| Target | pval | fstat | auc |
|---|---|---|---|
| TAGLN-558 | 2.899E−16 | 43.3106373 | 0.8653906 |
| FDCSP-324 | 5.7325E−16 | 42.3689005 | 0.8304306 |
| CASC15-411 | 2.2131E−11 | 28.272029 | 0.8139856 |
| CYP24A1-999 | 3.3734E−08 | 19.0579827 | 0.7838338 |
| FOXI3-AP329YP | 5.8109E−08 | 20.208492 | 0.842881 |
| LINC02167-899 | 6.583E−05 | 11.8462275 | 0.8529412 |
| FSTL1-496 | 8.8212E−05 | 9.7831222 | 0.7083333 |
| JPH3-053 | 0.00013379 | 10.4144239 | 0.6712531 |
| CCT8-229 | 0.00049823 | 8.2131811 | 0.6579832 |
| FMNL3-999 | 0.00061507 | 7.8728001 | 0.7312925 |
| MED16-325 | 0.00178688 | 6.7450669 | 0.6036451 |
| MT-ND3-875 | 0.00208263 | 6.4721304 | 0.5743702 |
| STRA6-621 | 0.00330579 | 6.0163736 | 0.6602286 |
| KIF1B-511 | 0.00392857 | 5.8547197 | 0.5863354 |
| MT-ND4-876 | 0.00460729 | 5.5907279 | 0.6048456 |
| SOX11-386 | 0.00781285 | 5.014586 | 0.5717365 |
| EDN1-961 | 0.00876713 | 4.8478986 | 0.6504202 |
| WARS-737 | 0.01327487 | 4.5224258 | 0.627972 |
| SLCO1A2-483 | 0.01807084 | 4.2021936 | 0.7164125 |
| ATPAF2-017 | 0.01999229 | 4.0780507 | 0.6026442 |
| MT-ND1-873 | 0.03422241 | 3.4767862 | 0.5025709 |
| ATG4B-774 | 0.05466903 | 2.9762316 | 0.5798308 |
| MUC5B-588 | 0.09271689 | 2.4688079 | 0.6399371 |
| WIPI1-447 | 0.1658801 | 1.823655 | 0.5508575 |
| HERC4-778 | 0.1693294 | 1.7928819 | 0.5528501 |
| PUM2-354 | 0.2106277 | 1.5961968 | 0.581761 |
| COL17A1-053 | 0.3968551 | 0.9361489 | 0.6087302 |
| METTL9-590 | 0.4546682 | 0.7926098 | 0.5161921 |
| EAF1-532 | 0.4649112 | 0.7707373 | 0.5539326 |
| CDV3-555 | 0.4699621 | 0.7640828 | 0.5998428 |
| TIMP2-317 | 0.5112102 | 0.6734008 | 0.4922577 |
| ZIC2-845 | 0.5305922 | 0.6373613 | 0.5394506 |
| AKR1C1-886 | 0.5448283 | 0.6111425 | 0.5145019 |
| HLA-H-683 | 0.7366756 | 0.307049 | 0.5153846 |
| MT-CYB-AP473JM | 0.8804494 | 0.1276612 | 0.5326797 |
| SLC5A10-293 | 0.883739 | 0.1238363 | 0.5399698 |
| KRT7-840 | 0.9541557 | 0.046964 | 0.4990079 |

TABLE 9

| Target | pval | fstat | auc |
|---|---|---|---|
| MT-ND3-875 | 0.00124773 | 7.19106058 | 0.6550039 |
| JPH3-053 | 0.00215415 | 6.92605452 | 0.5311828 |
| TIMP2-317 | 0.00412691 | 5.69477289 | 0.6345515 |
| MT-ND4-876 | 0.00550635 | 5.47363813 | 0.6438889 |
| SOX11-386 | 0.00732225 | 5.13712242 | 0.6699554 |
| MED16-325 | 0.00757479 | 5.28172774 | 0.4273362 |
| KIF1B-511 | 0.01273312 | 4.66048809 | 0.5483516 |
| MT-ND1-873 | 0.01500524 | 4.4112506 | 0.5754067 |
| COL17A1-053 | 0.02379573 | 3.98114097 | 0.7085714 |
| HERC4-778 | 0.03262978 | 3.49973266 | 0.6368421 |
| AKR1C1-886 | 0.05137704 | 3.1258211 | 0.6303779 |
| TAGLN-558 | 0.06767604 | 2.74035929 | 0.5902134 |
| SLCO1A2-483 | 0.07706485 | 2.70497816 | 0.7072214 |
| ZIC2-845 | 0.07981327 | 2.60570002 | 0.6103787 |
| LINC02167-899 | 0.10403171 | 2.43660975 | 0.7921569 |
| ATPAF2-017 | 0.12347426 | 2.16511701 | 0.5167874 |
| PUM2-354 | 0.25023377 | 1.4244631 | 0.5910448 |
| CYP24A1-999 | 0.33860477 | 1.09134166 | 0.4863312 |
| CCT8-229 | 0.37974511 | 0.9823836 | 0.4837725 |
| MT-CYB-AP473JM | 0.3842415 | 0.98661221 | 0.6490196 |
| WARS-737 | 0.38941877 | 0.95736001 | 0.5621843 |
| METTL9-590 | 0.41028085 | 0.8985891 | 0.5154478 |
| FOXI3-AP329YP | 0.50178841 | 0.69829522 | 0.7340839 |
| EDN1-961 | 0.50858273 | 0.67887221 | 0.5548704 |
| MUC5B-588 | 0.51489487 | 0.67268347 | 0.4865672 |
| WIPI1-447 | 0.5159263 | 0.66691023 | 0.5005807 |
| HLA-H-683 | 0.67276489 | 0.39958286 | 0.528799 |
| CDV3-555 | 0.69252524 | 0.37012368 | 0.541791 |
| FMNL3-999 | 0.70472404 | 0.35115237 | 0.5940984 |
| STRA6-621 | 0.74099475 | 0.30084697 | 0.5953594 |
| CASC15-411 | 0.78491149 | 0.24263261 | 0.6720635 |
| FSTL1-496 | 0.78559104 | 0.24167221 | 0.6143791 |
| ATG4B-774 | 0.86972111 | 0.13980947 | 0.5588023 |
| KRT7-840 | 0.9431193 | 0.05863877 | 0.5249231 |
| FDCSP-324 | 0.99181488 | 0.00821924 | 0.6180708 |
| EAF1-532 | 0.99232303 | 0.00770728 | 0.5227273 |
| SLC5A10-293 | 0.99357864 | 0.00644293 | 0.5643939 |

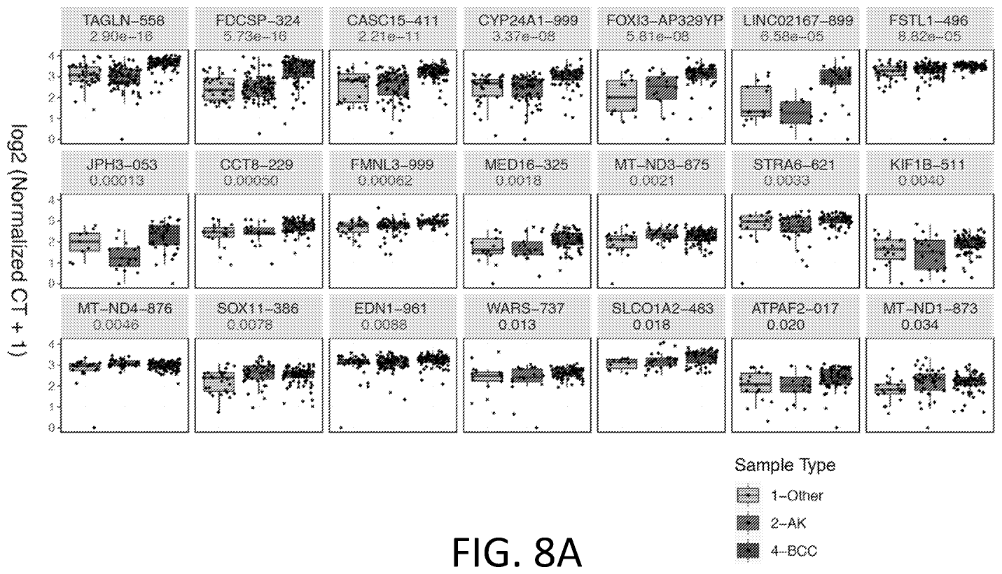

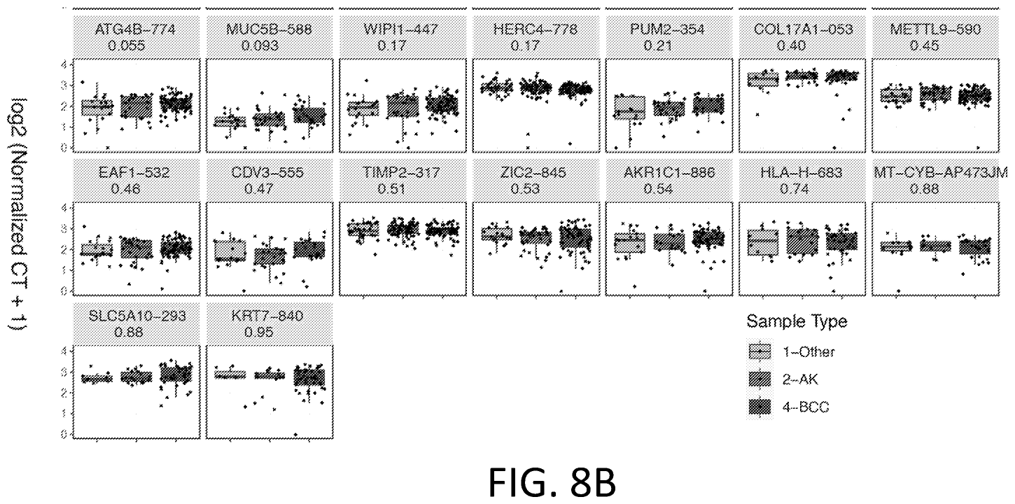

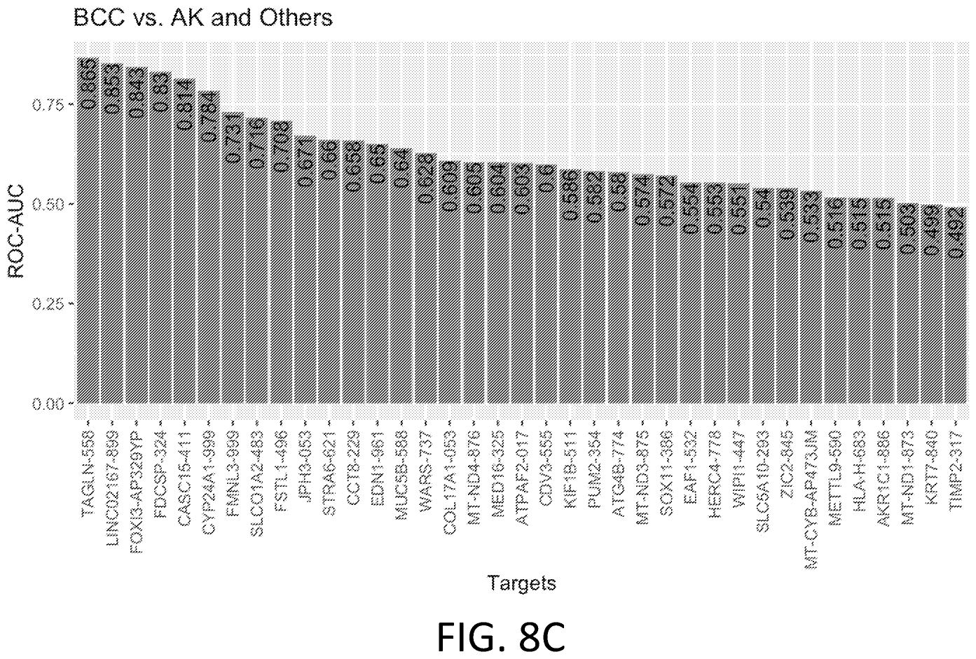

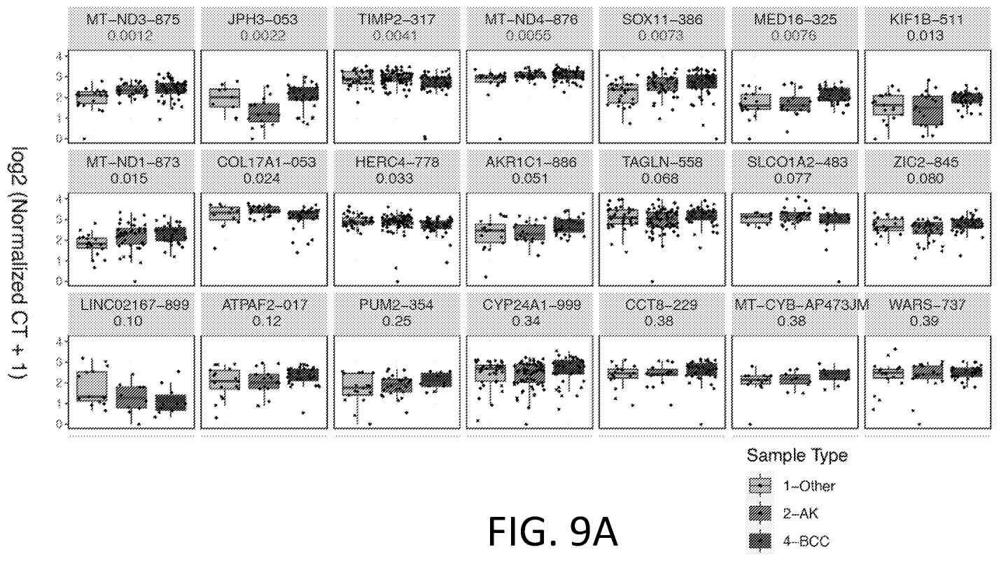

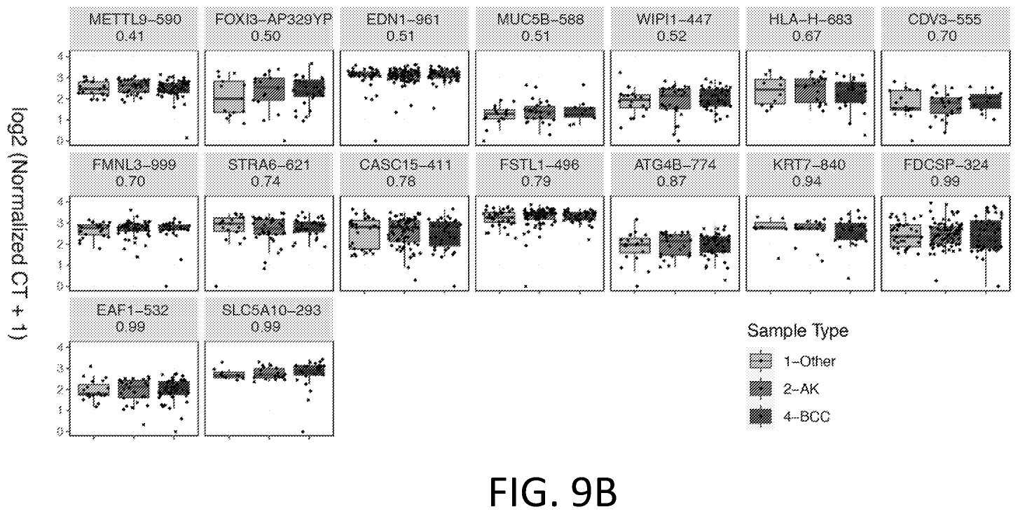

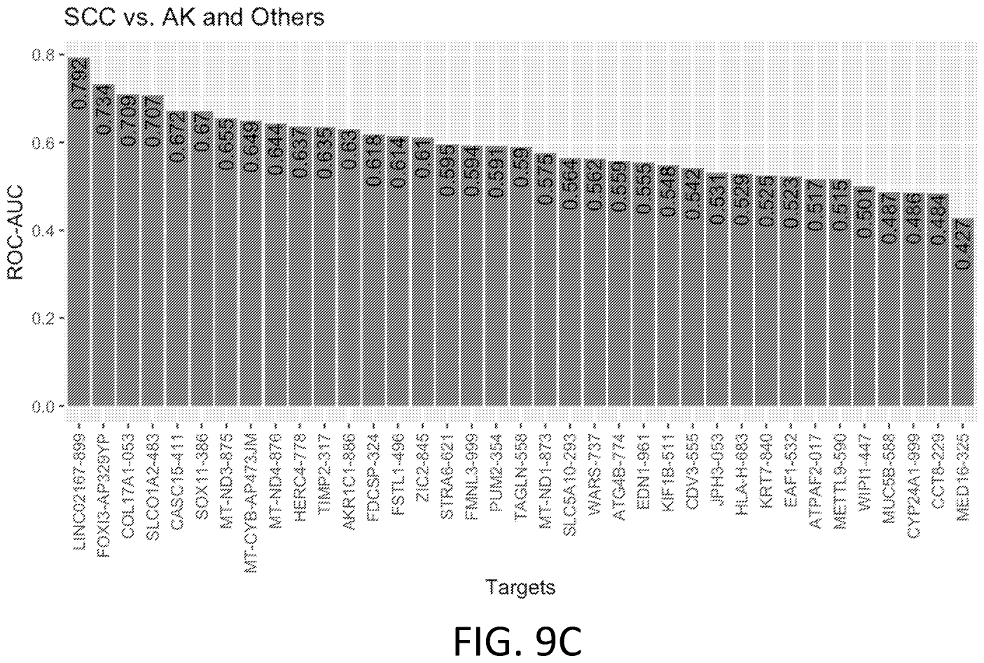

FIGS. 8A-8C shows the expression profiles of the genes that significantly (p-value<=0.01) classified BCC from AK/Other. FIGS. 9A-9C shows the expression profiles of the genes that significantly (p-value<=0.01) classified SCC from AK/Other. Other diseases are shown in Table 10.

TABLE 10
Skin Disorders and Diseases

Acantholytic Dyskeratotic acanthoma
Acanthoma Fissuratum
Angiofibroma
Benign Keratosis
Benign Adnexal Neoplasm with Follicular Differentiation
Benign Keratosis, ulcerated and inflamed
Benign Lichenoid Keratosis
Benign keratosis/Seborrheic keratosis, mildly inflamed
Benign lichenoid keratosis
Benign stasis dermatitis
Benign verrucous keratosis
Chronic Folliculitis
Consistent with Fibrous Papule
Consistent with Lichen Simplex Chronicus
Dermal Fibrosis and Vascular Telangiectasia
Dermal Scar
Dermatofibroma with Lentiginous epidermal hyperplasia and basilar
hypermelanosis
Epidermal Cyst
Epidermal Inclusion Cyst at the site of a Seborrheic Keratosis
Epidermal and follicular acanthosis with lichenoid lymphocytic
inflammation and an endophytic
component
Epidermal inclusion cyst
Eroded and inflamed Benign Keratosis
Fibrosing Granulation Tissue/Seborrheic Keratosis
Fibrous papule
Fibrous papule of the nose
Flat Seborrheic Keratosis
Granulomatous dermatitis
Hypertrophic scar
Inflamed Seborrheic Keratosis
Inflamed and irritated verrucous keratosis
Inflamed seborrheic keratosis
Inflamed verrucous keratosis
Inflammation, scar and regeneration
Irritated Benign Keratosis with Lichenoid Inflammation
Keloidal Scar
Lichen simplex chronicus
Lichenoid Keratosis
Nodular acute and chronic lymphohistiocytic inflammation
Perifollicular non-caseating granulomatous inflammation with
multinucleated giant cells
Porokeratosis
Psoriasiform dermatitis with focal intraepidermal atypical lymphocytes
Psoriasiform and Spongiotic dermatitis
Reticulated Seborrheic Keratosis
Scar
Scar with regeneration and repair
Scar with stasis changes
Sebaceous hyperplasia
Seborrheic Keratosis
Seborrheic Keratosis, clonal type
Seborrheic Keratosis, inflamed and irritated
Seborrheic Keratosis, pigmented and inflamed
Seborrheic keratosis, inflamed
Solar Lentigo
Spongiotic dermatitis
Spongiotic dermatitis and scar
Traumatized erosion with repair
Tumor of Follicular infundibulum
Verruca Vulgaris
Verruca Vulgaris Inflamed
Verrucous Keratosis, traumatized and inflamed
Verrucous keratosis
Warty dyskeratoma, benign

### Example 3

### Identification of Novel Gene Classifiers to Non-Invasively Diagnose Non-Melanoma Skin Cancer

Non-melanoma skin cancers (NMSC) are the most common types of skin cancer and include both basal cell carcinoma (BCC) and squamous cell carcinoma (SCC). NMSC primarily form on sun exposed skin including the head, face, neck, arms, and hands. BCC accounts for >75% of NMSC cases; however, SCC is more aggressive and may occur in other locations as well. Combined, BCC and SCC are responsible for >15,000 deaths each year in the US alone, which exceed deaths due to melanoma. Current diagnosis of NMSC generally relies on an in-depth visual assessment of the lesion in question followed by a surgical skin biopsy for histopathologic review. This example used non-invasive collection of skin tissue with ‘smart stickers’ and subsequent genomic analysis to classify NMSC. Adhesive skin collections kits were used to collect the lesional skin from 58 patients with BCC, 41 patients with SCC, and 42 patients with non-cancerous skin diseases. Whole transcriptomic analysis was conducted on each sample and differentially expressed genes were determined by comparing BCC and/or SCC with non-cancerous skin disease (other) using multiple comparisons. Eighteen genes were significantly (fold change>1.5; p<0.1) increased in BCC compared to other skin diseases while 14 genes were increased in SCC (fold change>1.5; p<0.1). Further analysis identified 12 genes that were differentially expressed in both lesional BCC and lesional SCC compared to other skin diseases. These results indicated non-invasive skin sampling and genomic analysis provides an opportunity to identify patients with NMSC earlier and without the need for surgical biopsy.

Subjects. Subjects at least 18 years of age with clinically suspected basal cell carcinoma (BCC) or squamous cell carcinoma (SCC) lesions were enrolled in this study. Non-invasive skin samples were collected from all enrolled subjects using the DermTech adhesive skin collection kit (DermTech, Inc.; La Jolla, CA) as described below. Additionally, skin biopsies were collected from all enrolled subjects for histopathological confirmation. The study was reviewed and approved by Aspire IRB (Santee, CA). All subjects provided written consent prior to enrollment. Histopathologically confirmed subjects consisted of basal cell carcinoma (N=48); squamous cell carcinoma (N=41); and “other” (Subjects were classified as “other” if histopathology confirmed their diagnosis as a non-cancerous skin disease including but not limited to seborrheic keratosis, actinic keratosis, verruca vulgaris, and plaque psoriasis).

Skin Sampling. The DermTech adhesive skin collection kit (DermTech, La Jolla, CA) was used to collect skin samples from lesional skin and nearby non-lesional skin. Prior to application of the Smart Sticker™, the target skin was prepped with an alcohol pad to remove oils and then dry wicked with a gauze pad to remove any remaining moisture. Each kit contains a total of 4 Smart Stickers™ for sample collection. Smart Stickers™ were applied individually to the designated area and 5 circular motions with the thumb were used to ensure adhesion to the targeted skin. Using a pen, small marks were made on the skin after placement of the first tape-strip to ensure consistent placement of each subsequent tape-strip. Smart Stickers™ were removed slowly using standard precautions to prevent folding and eliminate potential sources of contamination. Once removed, the Smart Sticker™ was placed onto the tri-fold sample collector. This process was repeated with the 2^{nd}, 3^{rd}, and 4^{th} Smart Sticker™ on the same lesional/non-lesional skin. Once all 4 Smart Stickers™ were in the tri-fold collector, the collector was carefully folded and placed in a re-sealable bag for overnight shipment. Upon arrival at DermTech, samples were stored at −70° C. or colder until RNA extraction.

RNA Extraction and Gene Expression Analysis. RNA was extracted from Smart Stickers™ using a closed-tube, bead-based method. Briefly, Smart Stickers™ were enzymatically digested to extract the genomic material from the adhesive and nucleic acid isolated using magnetic beads on a kingfisher flex instrument (ThermoFisher Scientific). The whole transcriptomic analysis library was prepared in house using the ‘SMART-Seq® Stranded Kit’ (Takara Bio), and all sequencing was performed on the Illumina HiSeq x Ten.

Statistical Analysis. The identification of differentially expressed genes among the disease types was performed by the R package “DeSeq2”. P values were adjusted for multiple comparisons by the Benjamini-Hochberg method among the 54k genes analyzed, using an adjusted P-value threshold of 0.1. Top genes were chosen based on sorted adjusted P values. Dotplots and heatmaps were based on the top genes.

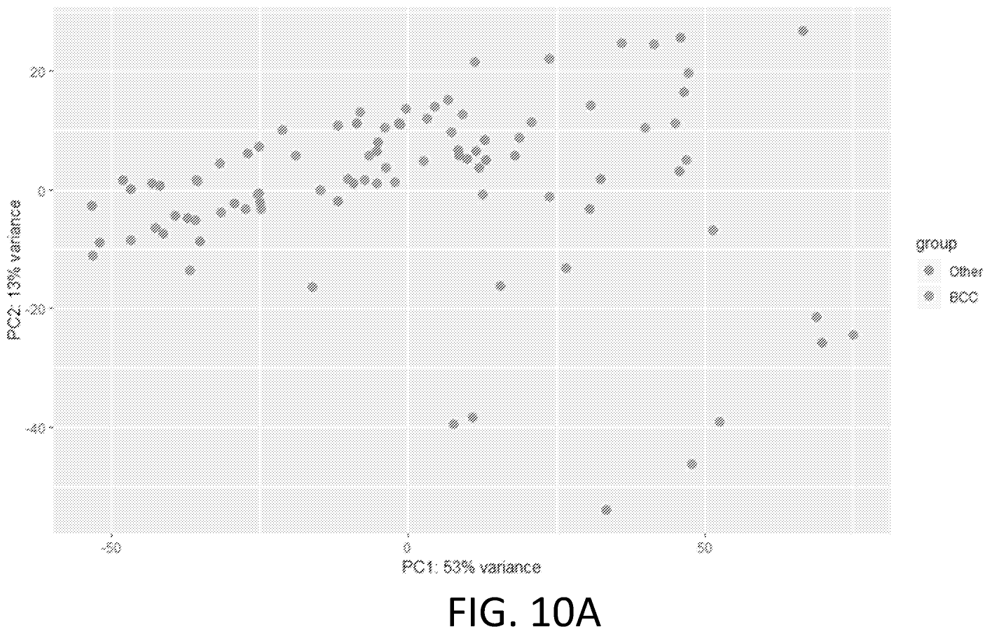

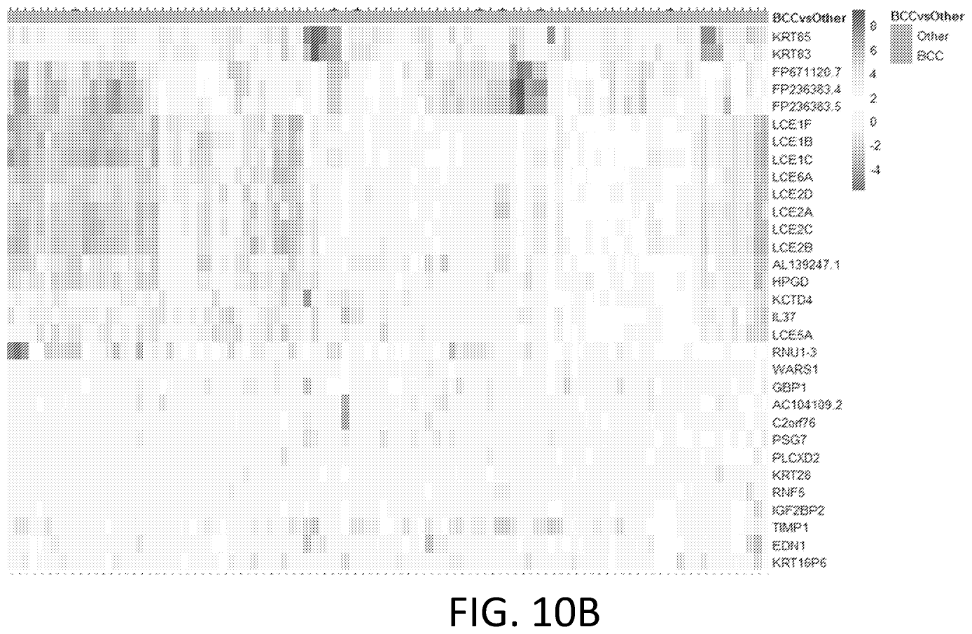

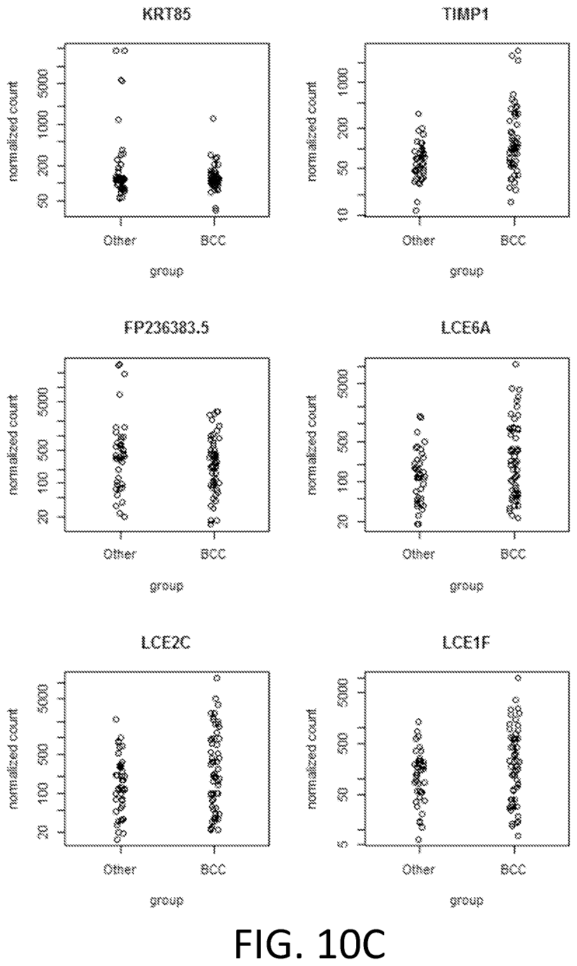

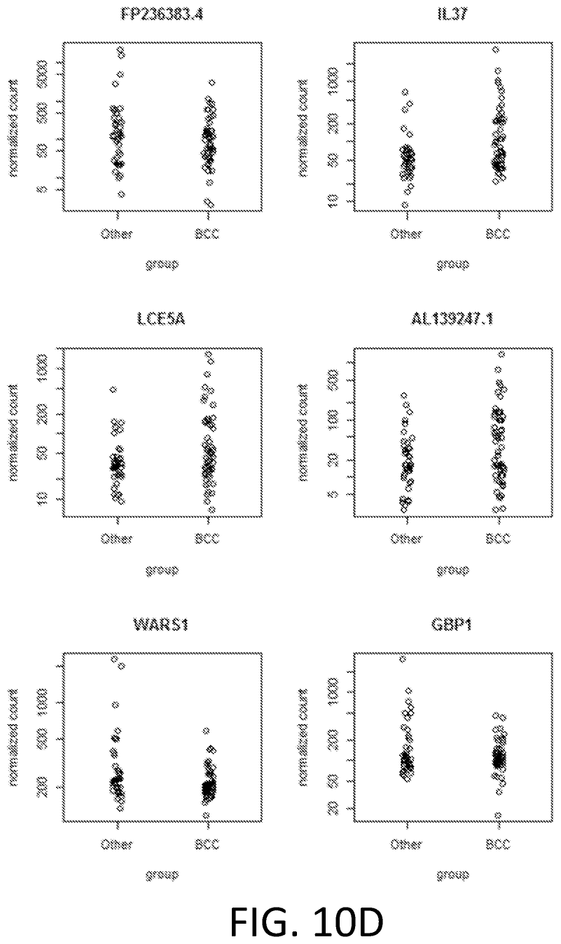

Results. Approximately one third of subjects enrolled in this study were incorrectly clinically diagnosed as BCC or SCC, suggesting the importance of additional objective assessments (e.g., non-invasive sampling) in ruling out non-melanoma skin cancer prior to a surgical biopsy. Non-invasively assessment of BCC or SCC lesions identified gene signatures capable of distinguishing non-melanoma skin cancer from non-cancer skin inflammation. Results for the top genes differentiating basal cell carcinoma from other skin diseases are shown in FIGS. 10A-10D. Top genes for BCC included KRT85, TIMP1, FP236383.5, LCE6A, LCE2C, LCE1F, FP236383.4, IL37, LCE5A, AL139247.1, WARS1, and GBP1. Results for the top genes differentiating squamous cell carcinoma from other skin diseases are shown in FIGS. 11A-11C. Top genes for SCC included KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1.

### Example 4

### Classifiers for Non-Melanoma Skin Cancer Detection and Diagnosis

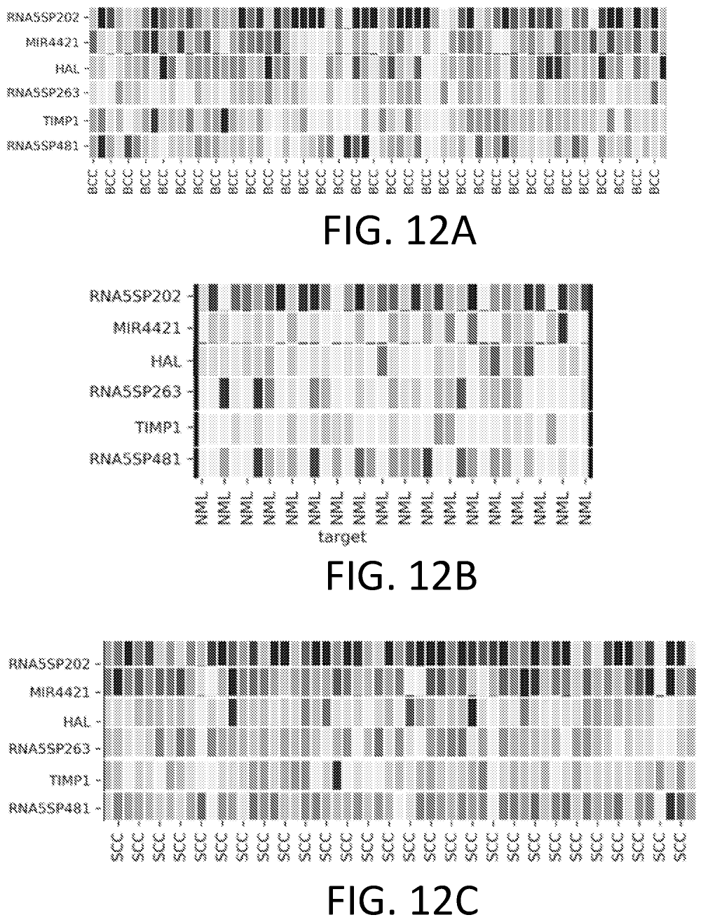

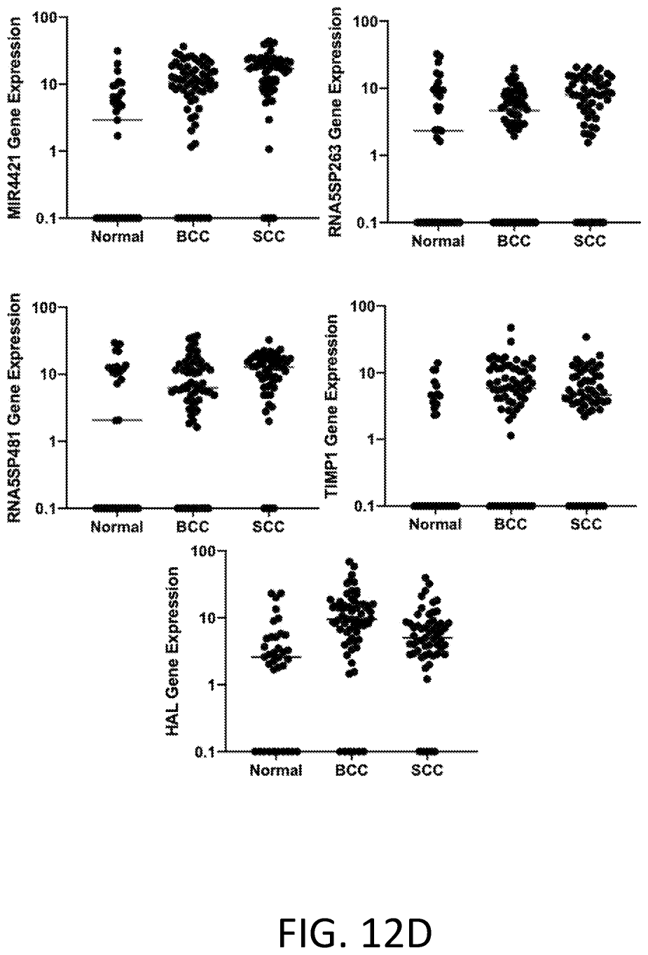

Non-invasive skin samples were collected from normal skin of healthy controls (n=39), lesional basal cell carcinoma (n=67), lesional squamous cell carcinoma (n=57), and lesions from other skin diseases (n=58). Skin samples were processed as previously described (see DermTech SOP's) and RNA was analyzed by whole transcriptomic analysis. Data collected was analyzed using a combination of machine learning/artificial intelligence as well as comparing groups to identify differentially expressed genes. Each approach generated a list of genes which could be used to stratify basal cell carcinoma or squamous cell carcinoma from either healthy skin or other skin diseases. By combining the datasets, six genes were identified for further investigation as shown in FIGS. 12A-12C. Using these genes, samples could be differentiated in patients with basal or squamous cell carcinoma from those that do not have either of these skin diseases. (FIG. 12D).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method for non-invasive gene expression analysis of one or more target genes, comprising:
receiving or obtaining data comprising measurements of RNA and/or DNA obtained from a skin sample using a non-invasive sampling method, the skin sample having been obtained from a subject suspected of having a one or more skin diseases, wherein the skin disease comprises SCC (squamous cell carcinoma) and/or BCC (basal cell carcinoma); and
applying one or more classifiers to the measurements to analyze gene expression of the one or mor target genes in the skin sample suspected of comprising the one or more skin diseases.

2. The method of claim 1, wherein SCC comprises squamous cell carcinoma in situ (isSCC) or invasive squamous cell carcinoma (ivSCC).

3. The method of claim 1, wherein the non-invasive sampling method comprises adhering one or more adhesive patches to a skin area of the subject.

4. The method of claim 3, wherein the one or more adhesive patches are applied to the same area of skin of the subject.

5. The method of claim 3, wherein the one or more adhesive patches are applied to different areas of skin of the subject.

6. The method of claim 3, wherein the one or more adhesive patches are applied to about the same area of skin of the subject.

7. The method of claim 3, wherein the one or more adhesive patches are applied to a lesion.

8. The method of claim 1, wherein the DNA comprises gDNA or a complementary cDNA.

9. The method of claim 1, wherein the method comprises obtaining the measurements by performing RNA-sequencing (RNA-seq).

10. The method of claim 1, wherein the method comprises obtaining the measurements by RT-qPCR.

11. The method of claim 1, wherein the RNA comprises an mRNA.

12. The method of claim 11, wherein the mRNA encodes a protein that plays a role in one or more of keratinization, developmental biology, metabolism of vitamins or cofactors, neutrophil degranulation, NOD-like receptor signaling, or an innate immune system.

13. The method of claim 10 or 11, wherein the mRNA comprises transcripts of one or more of: MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1.

14. The method of claim 13, wherein the mRNA comprises transcripts of one or more of: MT-ND3, JPH3, TIMP2, MT-ND4, SOX11, and MED16.

15. The method of claim 13, wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1.

16. The method of claim 10 or 12, wherein the mRNA comprises a transcript of AD000090.1, CASP14, CXCL8, FABP5, IVNS1ABP, KRT1, KRT2, KRT5, KRT6C, NAMPT, or SLC2A3.

17. The method of claim 10 or 12, wherein the mRNA comprises a transcript of CAPZA2, IVNS1ABP, NCOA1, RAB5B, SCAF11, STK4, or TPM4.

18. The method of claim 10, wherein the mRNA is indicative of SCC.

19. The method of claim 18, wherein the expression level of a gene encoded by the mRNA is increased relative to a control.

20. The method of claim 18, wherein the expression level of a gene encoded by the mRNA is increased at least 1.5 fold relative to a control.

21. The method of claim 18, wherein the expression level of a gene encoded by the mRNA has a p value of no more than 0.1 relative to a control.

22. The method of claim 18, wherein the expression level of a gene encoded by the mRNA has a p value of no more than 0.01 relative to a control.

23. The method of claim 18, wherein the SCC comprises squamous cell carcinoma in situ (isSCC) and/or invasive squamous cell carcinoma (ivSCC).

24. The method of claim 1, wherein the method further comprises providing an SCC treatment or an actinic keratosis (AK) treatment to a patient diagnosed with SCC or AK, respectively.

25. The method of claim 10 or 12, wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1.

26. The method of claim 25, wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1.

27. The method of claim 25, wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1.

28. The method of claim 25, wherein the mRNA comprises transcripts of one or more of RNA5SP202, MIR4421, HAL, RNA5SP263, TIMP1, and RNA5SP481.

29. The method of claim 1, wherein the classifier is trained using deep learning, a hierarchical cluster analysis, a principal component analysis, a partial least squares discriminant analysis, a random forest classification analysis, a support vector machine analysis, a k-nearest neighbors analysis, a naive Bayes analysis, a K-means clustering analysis, or a hidden Markov analysis.

30. The method of claim 1, wherein the method further comprises diagnosing a patient identified as having the skin disease.

31. The method of claim 30, wherein the method further comprises providing a treatment to the subject diagnosed with the skin disease.

32. The method of claim 31, wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications.

33. The method of claim 32, wherein topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod.

34. The method of claim 32, wherein immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

35. A method for non-invasive gene expression analysis of one or more target genes, comprising:
receiving or obtaining data comprising measurements of RNA and/or DNA obtained from a skin sample using a non-invasive sampling method, the skin sample having been obtained from a subject suspected of having SCC, wherein the SCC comprises squamous cell carcinoma in situ (isSCC) or invasive squamous cell carcinoma (ivSCC); and
applying a classifier to the measurements to analyze gene expression of the one or more target genes in the skin sample suspected of comprising isSCC or ivSCC.

36. The method of claim 35, the method comprising obtaining the skin sample by adhering the adhesive patch to a skin area of the subject.

37. The method of claim 35, wherein the DNA comprises gDNA or a complementary cDNA.

38. The method of claim 35, comprising obtaining the measurements by performing RNA-sequencing (RNA-seq).

39. The method of claim 35, wherein the method comprises obtaining the measurements by RT-qPCR.

40. The method of claim 35, wherein the RNA comprises mRNA.

41. The method of claim 40, wherein the mRNA comprises transcripts of one or more of: AC105460.1, ARC, C5AR1, COL6A1, CYFIP1, DDX3X, EDN1, EFCAB2, FAM110C, FOSL1, G0S2, IL36G, ITGA3, JUN, KRTAP1-3, KRTAP1-5, KRTAP3-1, KRTAP4-6, KRTAP9-4, MAGED1, MT-CO2, PIM1, RN7SL752P, SERPINA1, SLC2A3, SPRR2A, SRGN, or U3.

42. The method of claim 40, wherein the mRNA comprises a transcript of AC105460.1, ARC, COL6A1, EDN1, FAM110C, FOSL1, IL36G, JUN, PIM1, or RN7SL752P.

43. The method of claim 40, wherein the mRNA comprises a transcript of CYFIP1, DDX3X, ITGA3, MAGED1, MT-CO2, or SPRR2A.

44. The method of claim 40, wherein the mRNA is indicative of isSCC.

45. The method of claim 40, wherein the mRNA comprises a transcript of C5AR1, G0S2, KRTAP1-3, KRTAP1-5, KRTAP3-1, KRTAP4-6, KRTAP9-4, SERPINA1, SLC2A3, or SRGN.

46. The method of claim 40, wherein the mRNA comprises a transcript of EFCAB2, KRTAP4-6, KRTAP9-4, or U3.

47. The method of claim 40, wherein the mRNA is indicative of ivSCC.

48. The method of claim 40, wherein the expression level of a gene encoded by the mRNA is increased at least 1.5 fold relative to a control.

49. The method of claim 40, wherein the gene encoded by the mRNA has a p-value of no more than 0.1 relative to a control.

50. The method of claim 40, wherein the gene encoded by the mRNA has a p-value of no more than 0.01 relative to a control.

51. The method of claim 40, wherein the method further comprises providing an isSCC treatment or an ivSCC treatment to a subject diagnosed with isSCC or ivSCC.

52. The method of claim 51, wherein the treatment comprises one or more of excisional surgery, Mohs surgery, cryosurgery, curettage and electrodesiccation (electrosurgery), laser surgery, radiation, immunotherapy, photodynamic therapy (pdt), and applying topical medications. In some instances, topical medications comprise 5-fluorouracil (5-FU), ingenol mebutate, diclofenac, retnin-A, topical non-steroidal anti-inflammatory drugs (NSAIDs), CAR-T, PD1 inhibitors, or imiquimod. In some embodiments, immunotherapy comprises treatment with aldesleukin, atezolizumab, avelumab, cemiplimab, dostarlimab, imiquimod, ipilimumab, nivolumab, peginterferon alfa-2b, pembrolizumab, poly ICLC, T-VEC, or a combination thereof.

53. A method for non-invasive differential gene expression analysis of one or more target genes between SCC and one or more different diseases, comprising:
receiving or obtaining data comprising measurements of RNA and/or DNA obtained from a skin sample using a non-invasive sampling method, the skin sample having been obtained from a subject suspected of having SCC (squamous cell carcinoma) or the one or more different diseases; and
applying one or more classifiers to the measurements to analyze gene expression of the one or more target genes in the skin sample indicative of SCC and/or the one or more different skin diseases.

54. The method of claim 53, wherein the one or more different diseases comprises AK.

55. The method of claim 53, wherein the one or more different diseases comprises one or more diseases in Table 10.

56. The method of claim 53, wherein the RNA comprises mRNA.

57. The method of claim 53, wherein the method comprises obtaining the measurements by RT-qPCR.

58. The method of claim 56, wherein at least one mRNA transcript in the sample comprising SCC is upregulated compared to a control sample comprising the different disease.

59. The method of claim 56, wherein the mRNA comprises a transcript of one or more of IVNS1ABP, RAB5B, CAPZA2, STK4, SCAF11, NCOA1, and CBL.

60. The method of claim 53, wherein at least one mRNA transcript in the sample comprising SCC is downregulated compared to a control sample comprising the different disease.

61. The method of claim 56, wherein at least one mRNA transcript in the sample comprising ivSCC is upregulated compared to a control sample comprising isSCC.

62. The method of claim 56, wherein the mRNA comprises transcripts of one or more of: MT-ND3, JPH3, TIMP2, MT-ND4, SOX11, MED16, KRT33A, KRTAP8-1, KRTAP19-5, KRTAP2-2, KRTAP11-1, KRTAP9-4, KRT33B, KRTAP1-5, KRTAP3-3, KRTAP2-1, KRTAP3-1, and KRTAP1-1.

63. The method of claim 56, wherein the mRNA comprises transcripts of one or more of: MT-ND3, JPH3, TIMP2, MT-ND4, SOX11, and MED16.

64. The method of claim 56, wherein the mRNA comprises transcripts of one or more of: KRT33A, KRTAP8.1, KRTAP19-5, KRTAP2-2, KRTAP11-1, RTAP9-4, KRT33B, KRTAP1-5, KRTAP3-3, KRTAP2-1, KRTAP3-1, and KRTAP1-1.

65. The method of claim 56, wherein the mRNA comprises a transcript of AD000090.1, CASP14, CXCL8, FABP5, IVNS1ABP, KRT1, KRT2, KRT5, KRT6C, NAMPT, or SLC2A3.

66. The method of claim 56, wherein the mRNA comprises a transcript of CAPZA2, IVNS1ABP, NCOA1, RAB5B, SCAF11, STK4, or TPM4.

67. The method of claim 56, wherein the mRNA comprises EFCAB2, KRTAP4-6, KRTAP9-4, and U3.

68. The method of claim 56, wherein the mRNA comprises transcripts of one or more of RNA5SP202, MIR4421, HAL, RNA5SP263, TIMP1, and RNA5SP481.

69. The method of claim 56, wherein at least one mRNA transcript in the sample comprising ivSCC is downregulated compared to a control sample comprising isSCC.

70. The method of claim 56, wherein the mRNA comprises a transcript of one or more of DDX3X, CYFIP1, ITGA3, MAGED1, SPRR2Am, MT-CO2.

71. The method of claim 56, wherein the expression level of a gene encoded by the mRNA is increased at least 1.5 fold relative to a control.

72. The method of claim 56, wherein the gene encoded by the mRNA has a p-value of no more than 0.1 relative to a control.

73. The method of claim 56, wherein the gene encoded by the mRNA has a p-value of no more than 0.01 relative to a control.

74. A method for non-invasive differential gene expression analysis of one or more target genes between basal cell carcinoma (BCC) and one or more different diseases, comprising:
receiving or obtaining data comprising measurements of RNA and/or DNA obtained from a skin sample using a non-invasive sampling method, the skin sample having been obtained from a subject suspected of having BCC (basal cell carcinoma) or the one or more different diseases; and
applying one or more classifiers to the measurements to analyze gene expression of the one or more target genes in the skin sample indicative of BCC and/or the one or more different skin diseases.

75. The method of claim 74, wherein the one or more different diseases comprises AK.

76. The method of claim 74, wherein the one or more different diseases comprises one or more diseases in Table 10.

77. The method of claim 74, wherein the RNA comprises mRNA.

78. The method of claim 73, wherein the method comprises obtaining the measurements by RT-qPCR.

79. The method of claim 74, wherein at least one mRNA transcript in the sample comprising BCC is upregulated compared to a control sample comprising the different disease.

80. The method of claim 77, wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, EDN1, KRT85, TIMP1, FP236383.5, LCE6A, LCE2C, LCE1F, FP236383.4, IL37, LCE5A, AL139247.1, WARS1, and GBP1.

81. The method of claim 77, wherein the mRNA comprises transcripts of one or more of: TAGLN, FDCSP, CASC15, CYP24A1, FOXI3, LINC02167, FSTL1, JPH3, CCT8, FMNL3, MED16, MT-ND3, STRA6, KIF1B, MT-ND4, SOX11, and EDN1.

82. The method of claim 77, wherein the mRNA comprises transcripts of one or more of: KRT85, TIMP1, FP236383.5, LCE6A, LCE2C, LCE1F, FP236383.4, IL37, LCE5A, AL139247.1, WARS1, and GBP1.

83. The method of claim 77, wherein the mRNA comprises a transcript of one or more of IVNS1ABP, RAB5B, CAPZA2, STK4, SCAF11, NCOA1, and CBL.

84. The method of claim 77, wherein the mRNA comprises transcripts of one or more of RNA5SP202, MIR4421, HAL, RNA5SP263, TIMP1, and RNA5SP481.

85. The method of claim 77, wherein at least one mRNA transcript in the sample comprising BCC is downregulated compared to a control sample comprising the different disease.

86. The method of claim 77, wherein the expression level of a gene encoded by the mRNA is increased at least 1.5 fold relative to a control.

87. The method of claim 77, wherein the expression level of a gene encoded by the mRNA has a p value of no more than 0.1 relative to a control.

88. The method of claim 77, wherein the expression level of a gene encoded by the mRNA has a p value of no more than 0.01 relative to a control.

89. A non-invasive system for analyzing a sample for differential gene expression, comprising:
a communication interface that receives data comprising measurements of RNAs over a communication network, the measurements having been obtained from a skin sample using a non-invasive sampling method, and the skin sample having been obtained from a subject suspected of having SCC comprises squamous cell carcinoma in situ (isSCC) or invasive squamous cell carcinoma (ivSCC); and
a computer in communication with the communication interface, wherein the computer comprises one or more computer processors and computer readable medium comprising machine-executable code that, upon execution by the one or more computer processors, implements a method comprising:
applying a classifier to the measurements to identify the skin sample indicative of the isSCC or ivSCC.

90. A non-invasive system for analyzing a sample for differential gene expression comprising:
providing a skin sample from a subject suspected of having one or more skin conditions, wherein the sample was obtained using a non-invasive sampling method;
enriching RNAs from the skin sample;
obtaining data comprising measurements from the RNAs;
determining expression levels for one or more genes from the measurements; and
applying a gene classifier to identify the skin sample comprises the one more skin conditions, wherein the skin disease comprises SCC (squamous cell carcinoma) BCC (basal cell carcinoma), AK (actinic keratosis), or a disease in Table 10.

91. A non-invasive system for analyzing a sample for differential gene expression, comprising
a communication interface that receives data comprising measurements of RNAs over a communication network, the measurements having been obtained from a skin sample using a non-invasive sampling method, and the skin sample having been obtained from a subject suspected of having one or more skin conditions; and
a computer in communication with the communication interface, wherein the computer comprises one or more computer processors and computer readable medium comprising machine-executable code that, upon execution by the one or more computer processors, implements a method comprising:
applying a classifier to the measurements to identify the skin sample indicative of the one or more skin conditions, wherein the skin disease comprises SCC (squamous cell carcinoma) BCC (basal cell carcinoma), AK (actinic keratosis), or a disease in Table 10.
